(19) United States
(12) Patent Application Publication
(10) Pub. No.: US 2023/0338374 A1
(43) Pub. Date: Oct. 26, 2023

(54) MDM2 INHIBITORS FOR USE IN THE TREATMENT OR PREVENTION OF HEMATOLOGIC NEOPLASM RELAPSE AFTER HEMATOPOIETIC CELL TRANSPLANTATION

(71) Applicant: Novartis AG, Basel (CH)

(72) Inventors: Robert Zeiser, Freiburg (DE); Justus Duyster, Freiburg (DE); Hans Dietrich Menssen, Basel (CH)

(21) Appl. No.: 18/026,972

(22) PCT Filed: Sep. 21, 2021
(86) PCT No.: PCT/EP2021/075896

§ 371 Date: Mar. 17, 2023

(30) Foreign Application Priority Data
Sep. 21, 2020 (EP) 20197230.4
Jul. 8, 2021 (EP) 21184448.5

(51) Int. Cl. A61K 31/506; A61K 31/496; A61P 35/02; A61K 35/14; A61K 45/06

(52) U.S. Cl. CPC A61K 31/506; A61K 31/496; A61P 35/02; A61K 35/14; A61K 45/06

(57) ABSTRACT

The invention relates to a mouse double minute 2 (MDM2) inhibitor for use in the treatment and/or prevention of a hematologic neoplasm relapse after hematopoietic cell transplantation (HCT) in a patient. In embodiments, the hematologic neoplasm is a leukaemia, preferably acute myeloid leukaemia (AML). Preferably, the patient received an allogeneic T cell transplantation, either together with the HCT and/or after HCT, such as at the time point of MDM2 administration. Furthermore, the invention relates to a pharmaceutical composition comprising a MDM2 inhibitor and an exportin 1 (XPO-1) inhibitor for use in the treatment and/or prevention of a hematologic neoplasm relapse after hematopoietic cell transplantation (HCT) in a patient according to any of the preceding claims

The invention relates to a mouse double minute 2 (MDM2) inhibitor for use in the treatment and/or prevention of a hematologic neoplasm relapse after hematopoietic cell transplantation (HCT) in a patient. In embodiments, the hematologic neoplasm is a leukaemia, preferably acute myeloid leukaemia (AML). Preferably, the patient received an allogeneic T cell transplantation, either together with the HCT and/or after HCT, such as at the time point of MDM2 administration. Furthermore, the invention relates to a pharmaceutical composition comprising a MDM2 inhibitor and an exportin 1 (XPO-1) inhibitor for use in the treatment and/or prevention of a hematologic neoplasm relapse after hematopoietic cell transplantation (HCT) in a patient according to any of the preceding claims.

### BACKGROUND OF THE INVENTION

Acute myeloid leukemia (AML) relapse is the major cause of death after allogeneic hematopoietic cell transplantation (allo-HCT) after day 100 post-transplant (1). Major mechanisms promoting relapse include downregulation of MHC class II (MHC-II) (2,3), loss of mismatched HLA4, upregulation of immune checkpoint ligands (3), and reduced IL-15 production (5) and leukemia-derived lactic acid release (6) among others (reviewed in 7). Downregulation of pro-apoptotic genes including TNF-related apoptosis-inducing ligand (TRAIL) receptor 1 and 2 was shown to be connected to therapy-resistance and relapse in AML (8). These data suggest that approaches that increase MHC-II or TRAIL-R1/2 expression could be successful to treat AML relapse post allo-HCT.

Current pharmacological approaches for AML relapse include besides other FLT3 kinase inhibitors, immune checkpoint inhibitors, demethylating agents, bcl-2 inhibitors and others (reviewed in 9). Mouse double minute-2 (MDM2) inhibitors (10,11) can induce p53-dependent apoptosis in AML, however their role in the post allo-HCT setting has not been evaluated so far.

In light of the prior art there remains a significant need in the art to provide additional and/or improved means for treating leukemia or lymphoma relapse and in particular AML relapse after HCT. In particular, such a treatment could encompass compounds that increase MHC-II or TRAIL-R1/2 expression in leukemia cells. However, such compounds are not available to date. and there remains a need for provision of such compounds.

### SUMMARY OF THE INVENTION

In light of the prior art the technical problem underlying the present invention is to provide alternative and/or improved means for treating leukemia or lymphoma relapse and in particular AML relapse after HCT. Such means should include compounds, molecules and/or compositions suitable for mediating upregulation or maintaining expression of MHC-II or TRAIL-R1/2 expression in leukemia cells.

This problem is solved by the features of the independent claims. Preferred embodiments of the present invention are provided by the dependent claims.

The invention therefore relates to a mouse double minute 2 (MDM2) inhibitor for use in the treatment and/or prevention of a hematologic neoplasm relapse after hematopoietic cell transplantation (HCT) in a patient. The MDM2 inhibitor may be administered before and/or at the same time as and/or after administration of the HCT (preferably after the HCT).

The invention is based on the entirely surprising finding that recurrence of cancer cells in a patient suffering from a hematological neoplasm after HCT can be specifically treated or prevented by administration of an MDM2 inhibitor. The invention goes back to the unexpected discovery that inhibition of MDM2 leads to an upregulation of MHC-I and MHC-II molecules in cancer cells, such as leukemia cells or AML cells, as well as of TRAIL-receptors. This leads to a massive enhancement of recognition of cancer cells of the patient by allogeneic T cells that have been introduced into the patient with the HCT and/or with a separate transplantation of allogeneic T cells (allogeneic donor lymphocyte infusion; DLI). In other words, exposure to MDM2 inhibitors make cancer cells of the patient immunologically “visible” or strongly enhances the immunologic “visibility” so that the grafted allogeneic T cells can now recognize and attack the cancer cells.

The MDM2 protein functions as an ubiquitin ligase that recognizes the N-terminal trans-activation domain of p53 and as an inhibitor of p53 transcriptional activation. Mdm2 overexpression, in cooperation with oncogenic Ras, promotes transformation of primary rodent fibroblasts, and MDM2 inhibition can increase p53 activity (11). The MDM2 effects are via reducing p53 protein levels, which promotes the accumulation of de novo mutations in tumor cells thereby enhancing their malignant potential. Besides its anti-oncogenic effect, p53 can increase the expression of certain immune-related genes. In the context of the present invention, it has been surprisingly found that similar mechanisms are operational in cancer cells of hematological neoplasms, and in particular in AML cells, namely upregulation of HLA-class II molecules and TRAIL-receptors, rendering them more susceptible for alloreactive donor T cell response after allo-HCT.

It was completely unexpected that MDM2 inhibition causes TRAIL-R1/2 expression in leukemia and lymphoma cells, such as primary human AML cells and AML cell lines. Upon TRAIL ligation, TRAIL death receptors assemble at their intracellular death domain (DD), the death-inducing-signaling-complex (DISC) composed of FAS-associated protein with death domain (FADD) and pro-caspase-8/10 (17). TRAIL-R activation was shown to have anti-tumor activity (18).

Furthermore, it was discovered herein that MDM2 inhibition also increased MHC-II expression on primary leukemia and lymphoma cells, in particular on human AML cells, which could offer a pharmacological intervention to reverse the MHC-II decrease observed in AML relapse after allo-HCT (2, 3).

In embodiments, the hematologic neoplasm is selected from the group comprising leukemias, lymphomas and myelodysplastic syndromes. In embodiments, the hematologic neoplasm is a leukemia, preferably acute myeloid leukemia (AML).

In embodiments, the hematologic neoplasm comprises one or more mutations, such as an oncogenic mutation, which induce MDM2 and/or MDM4 expression in the neoplastic cells.

Surprisingly, certain mutations induce MDM2 and/or MDM4, which renders such neoplastic cancer cells particularly susceptible to treatment with MDM2 inhibitors. In preferred embodiments, the hematologic neoplasm comprising one or more MDM2 and/or MDM4 inducing mutations is AML. A MDM2 and/or MDM4 inducing mutation can be, for example, a point mutation or a fusion gene, which can be formed through chromosomal translocation.

The MDM2 and/or MDM4 inducing mutation can be selected, without limitation, from the group comprising cKit-D816V, FIP1L-PDGFR-α, FLT3-ITD, and JAK2-V617F. Further MDM2 and/or MDM4 inducing mutations can be identified, for example by using the techniques described herein.

cKit-D816V is an activating mutation of codon 816 of the Kit gene which is implicated in malignant cell growth in particular in acute myeloid leukemia (AML), but also in systemic mastocytosis and germ cell tumors, which is characterized by a substitution of aspartic acid with valine (D816V) and which renders the receptor independent of ligand for activation and signaling.

FIP1L1-PDGFRα fusion genes have been detected in the eosinophils, neutrophils, mast cells, monocytes, T lymphocytes, and B lymphocytes involved in hematological malignancies, in particular in AML. FIP1L1-PDGFR-α fusion proteins retain PDGFR-α-related Tyrosine kinase activity but, unlike PDGFR-α, their tyrosine kinase is constitutive, i.e. continuously active: the fusion proteins lack the intact juxtamembrane domain of PDGFR-α which normally blocks tyrosine kinase activity unless PDGFR-α is bound to its activating ligand, platelet-derived growth factor. FIP1 L1-PDGFR-α fusion proteins are also resistant to PDGFR-α's normal pathway of degradation, i.e. Proteasome-dependent ubiquitnation. In consequence, they are highly stable, long-lived, unregulated, and continuously express the stimulating actions of their PDGFRA tyrosine kinase component.

Treatment of a hematopoietic neoplasm relapse, such as AML relapse, after HCT with MDM2 inhibitors, preferably in combination with allogeneic T cell transplantation, is particularly efficient in patients with a neoplasm carrying MDM2 and/or MDM4 inducing mutations. Accordingly, in preferred embodiments the patients are known to suffer from a hematopoietic neoplasm carrying such mutations, as for example FLT3-ITD, JAK2-V617F, cKit-D816V or FIP1 L-PDGFR-α.

In embodiments, the HCT is an allogeneic HCT. It is preferable that the hematopoietic cell transplant is allogeneic (and is most preferable not T cell depleted), since due to the difference with respect to HLA molecules the allogeneic T cells comprised by the transplant can generate a graft versus leukemia or graft versus cancer cell response that is directed against cancer cells recurring after HCT. Accordingly, the MDM2 inhibitor administration can lead to a stronger anti-cancer effect of the engrafted T cells against cancer cells and can prevent recurrence of the cancer after HCT or can lead to control or eradication of the cancer cells after a relapse has occurred.

In embodiments, the HCT comprises T cells.

In embodiments, the MDM2 inhibitor is administered to a patient after HCT and before occurrence of a relapse. In the context of the present invention, the MDM2 inhibitor can be administered to the patient at various time points. For example, the inhibitor may be administered at the time point of HCT (time point of transplantation of the hematopoietic cells), such as on the same day. In embodiments, it may be useful to already administer the inhibitor before HCT, such as 1, 2, 3, 4, 5, 6 or 7 days before HCT, so that remaining cancer cells are immediately visible to the T cells comprised in the hematopoietic cell transplant. The MDM2 inhibitor can also be administered after HCT, such as 1, 2, 3, 4, 5, 6, 7, 8, 9, 10, 11, 12, 13, 14, 15, 16, 17, 18, 19, 20 or more days after HCT. In some embodiments, the MDM2 inhibitor is administered before, prior to, and after administration of the HCT. Preferably, the MDM2 inhibitor is administered (only) after the administration of the HCT.

MDM2 inhibitor administration can occur multiple times and even regularly repeated, such as daily, once every other day, once every 4 days, weekly, monthly, days 1-5 of a (repeated) 28 day schedule or days 1-7 of a (repeated) 28 day schedule.

MDM2 inhibitor administration can occur routinely in a patient with a hematological neoplasm who has received and/or is receiving and/or will receive HCT as a preventive measure, e.g. to enhance the graft versus cancer effect and to prevent occurrence of a cancer relapse in the patient.

In embodiments, the inhibitor is administered to a leukemia patient after occurrence of a relapse after HCT. The MDM2 inhibitor administration can be a therapeutic measure after occurrence of a relapse in a patient with a hematological neoplasm after HCT, potentially in combination with a further allogeneic T cell transplantation (preferably a donor lymphocyte infusion (DLI) that contains no hematopoietic stem cells).

In an embodiment, the MDM2 inhibitor is administered after the HCT, and a) before the allogenic T cell transplantation, and/or b) on the same day as the allogenic T cell transplantation, and/or c) after the allogenic T cell transplantation.

In this context it is understood that combinatorial administration of the MDM2 inhibitor and the allogeneic T cell transplantation can relate to a coordinated administration of the inhibitor and the cells. The two products do not have to be administered in a single composition but can be administered as separate compositions, also at different time points. For example, the patient may receive first the MDM2 inhibitor to induce upregulation of for example TRAIL-R1, TRAIL-R2, human leukocyte antigen (HLA) class I molecules and HLA class II molecules and receive the T cell transplant later on, such as later on the same day, or 1, 2, 3, 4, 5, 6, 7, 8, 9, of 10 days later. However, the two products can also be administered at about the same time, meaning roughly within 8 hours, or the MDM2 inhibitor can be administered after the T cell transplant has been administered. In this context one or both of the products (MDM2 inhibitor or the T cell transplant) can be administered more than once to the patient in a coordinated way.

It is understood that in the context of the present invention the coordinated administration of MDM2 with a further product, such as HCT, an allogeneic T cell transplant, and/or an XPO-1 inhibitor relates to the administration of the MDM2 inhibitor and the other product in order to enhance the therapeutic or preventive effect of the inhibitor. A skilled person is able to select a suitable administration regime depending on the specific case of the patient receiving the MDM2 inhibitor, and to coordinate the respective administrations of the inhibitor and the other compounds/products. Additionally, it is likely that leukemias with certain mutations that induce MDM2 expression will respond particularly well, as it was observed that for example cKIT-D816V and FIP1L-PDGFR-α induced MDM2 and MDM4. Along these lines, it could be shown that allo-T-cell/MDM2-inhibitor combination after allo-HCT (bone marrow transplantation) was highly effective in mice carrying FIP1L-PDGFR-α-mutant and cKIT-D816V-mutant AML.

In embodiments, the treatment of the invention further comprises administration of an allogeneic T cell transplantation, either together with the HCT and/or after HCT. In embodiments, the allogenic T cell transplantation is a donor lymphocyte infusion that comprises lymphocytes but does not comprise hematopoietic stem cells. In embodiments, the donor of the allogenic T cell transplantation was also the donor of the HCT.

In the context of the invention, the MDM2 inhibitor is preferably selected from the group comprising RG7112 (R05045337), idasanutlin (RG7388), AMG-232 (KRT-232), APG-115, BI-907828, CGM097, siremadlin (HDM-201), and milademetan (DS-3032b) and pharmaceutically acceptable salts thereof. In an embodiment, the MDM2 inhibitor is siremadlin (HDM-201), or a pharmaceutically acceptable salt or co-crystal (e.g. succinic acid co-crystal or succinate salt) thereof.

Various MDM2 inhibitors are known in the art and multiple established assays for the identification of MDM2 inhibitors have been described and are under investigation for treating various conditions (Marina Konopleva et al. Leukemia. 2020 Jul. 10. doi: 10.1038/s41375-020-0949-z). However, the use of MDM2 inhibitors for specifically treating or preventing cancer relapse in a patient with a hematological neoplasm after HCT has never been described or suggested in the art. The advantages of such a treatment have never been described so far and are based on the entirely surprising finding that cancer cells of hematological neoplasms, such as leukemia cells, upregulate molecules that enhance recognition of the cancer cells by allogeneic T cells.

In embodiments, administration of the MDM2 inhibitor leads to upregulation of one or more of TNF-related apoptosis-inducing ligand receptor 1(TRAIL-R1), TRAIL-R2, human leukocyte antigen (HLA) class I molecules and HLA class II molecules. Accordingly, in embodiments inhibition of MDM2 leads to upregulation of one or more of TNF-related apoptosis-inducing ligand receptor 1(TRAIL-R1), TRAIL-R2, human leukocyte antigen (HLA) class I molecules and HLA class II molecules. In embodiments, upregulation of one or more of TNF-related apoptosis-inducing ligand receptor 1(TRAIL-R1), TRAIL-R2, human leukocyte antigen (HLA) class I molecules and HLA class II molecules, in particular upregulation of TRAIL-R1 and/or TRAIL-R2, is p53 dependent.

In embodiments, administration of the MDM2 inhibitor increases cytotoxicity of CD8+ allo-T cells towards cancer cells, wherein preferably cytotoxicity of CD8+ allo-T cells is at least partially dependent on interaction of TRAIL-R of the cancer cells and TRAIL-ligand (TRAIL-L) of the CD8+ allo-T cells.

In embodiments, administration of the MDM2 inhibitor increases a graft-versus-leukemia (GVL) or a graft-versus-lymphoma reaction, wherein preferably the graft-versus-leukemia reaction or the graft-versus-lymphoma reaction is mediated by CD8+ allo-T cells.

In embodiments, administration of the MDM2 inhibitor increases expression of one or more of perforin, CD107a, IFN-γ, TNF and CD69 by CD8+ allo-T cells. Thus, according to one aspect of the invention, there is hereby provided a method of increasing expression of one or more of perforin, CD107a, IFN-γ, TNF and CD69 by CD8+ allo-T cells, the method comprising administration of an MDM2 inhibitor (e.g. HDM201 or a pharmaceutically acceptable salt thereof) in combination with a HCT (e.g., allogenic HCT, e.g. comprising T cells).

In embodiments, administration of the MDM2 inhibitor induces features of longevity (as described in (13)) in T-cells, in particular in CD8+ T-cells, such as CD8+ allo-T cells. For example, in embodiments, transplanted CD8+ T-cells display high expression of Bcl-2 and/or IL-7R (CD127) in the context of MDM2 inhibition. Furthermore, in embodiments, administration of the MDM2 inhibitor induces CD8+ T-cells with a high antigen recall response (as defined for example in (12)), such as CD8+ T-cells lacking CD27. In embodiments, MDM2 inhibitor treatment induces a decrease in CD8+CD27+TIM3+ donor T-cells.

A further entirely unexpected finding of the present invention is that the administration of an MDM2 inhibitor does not only lead to upregulation of receptors and surface molecules on the cancer cells as described herein, but it can also induce an advantageous phenotype in the allogeneic T cells in the patient leading to a stronger cytotoxic effect of the T cells towards the cancer cells. Roughly speaking, the MDM2 inhibitor can induce a more cytotoxic phenotype in the CD8+ allo-T cells rendering them more “aggressive” towards recurring cancer cells. Thus, according to one aspect of the invention, there is hereby provided a method of inducing a more effective cytotoxic phenotype in CD8+ allo-T cells, the method comprising administration of an MDM2 inhibitor (e.g. HDM201 or a pharmaceutically acceptable salt thereof) in combination with a HCT (e.g., allogenic HCT, e.g. comprising T cells).

In embodiments, administration of the MDM2 inhibitor to a subject according to the present invention enhances glycolytic activity of T cells in vivo during the graft-versus-leukemia reaction. Accordingly, in embodiments MDM2 inhibition leads to an increase in glycolytic activity of T cells in a subject. Thus, according to one aspect of the invention, there is hereby provided a method of enhancing the glycolytic activity in CD8+ allo-T cells, the method comprising administration of an MDM2 inhibitor (e.g. HDM201 or a pharmaceutically acceptable salt thereof) in combination with a

HCT (e.g., allogenic HCT, e.g. comprising T cells).

As shown herein, MDM2 inhibition leads to an increase in glycolytic activity in T-cells, including cytotoxic T-cells, which is indicative of stronger T-cell activation and increased GVL-activity. In embodiments, MDM2 inhibitor treatment increases the activation of T-cells and/or increases GVL-activity of T-cells in a subject. T-cells may be endogenous or administered T-cells, preferably CD8+ allo-T cells. As shown in the examples below, MDM-inhibition of a subject induces an increase in glycolytic activity of the T-cells in said subject.

It was completely unexpected that administration of an MDM2 inhibitor in the context of the present invention induces a T cell phenotype with enhanced/increased glycolytic activity, further improving the cytotoxic activity of CD8+ allo-T cells.

In embodiments, the patient may additionally receive an exportin 1 (XPO-1) inhibitor. Accordingly, in embodiments, the invention relates to the MDM2 inhibitor for use according to the invention, wherein the treatment further comprises administration of an expeortin-1 (XPO-1) inhibitor.

As shown in the examples below, MDM2 inhibition in AML cells leads to an increased TRAIL-R1/2 expression and enhances GVL against AML cells, which can be a huge advantage in the context of the treatment of a patient in case of a relapse after HCT or to prevent a relapse after HCT. The molecule XPO-1 mediates export of p53 from the nucleus and it was surprisingly found that in certain cancerous cells XPO-1 reduced p53-induced TRAIL-R1/2/MHC-II production upon MDM2 inhibition. Accordingly, it is advantageous to additionally inhibit XPO-1 in the context of the present invention in order to maximize the effect of MDM2 inhibition. The MDM2 inhibitor and an XPO-1 inhibitor can be administered in a coordinated way as described above for the combined administration of an MDM2 inhibitor and a hematopoietic cell transplant or an allogeneic T cell transplant. The administration of the two inhibitors may occur individually or in form of a pharmaceutical product or composition comprising both inhibitors.

Therefore, the present invention also relates to a pharmaceutical composition comprising a MDM2 inhibitor and an exportin 1 (XPO-1) inhibitor for use in the treatment and/or prevention of a hematologic neoplasm relapse after hematopoietic cell transplantation (HCT) in a patient according to any of the preceding claims. Such a pharmaceutical composition can be used in the context of all embodiments described herein.

Further, according to one aspect of the invention, there is hereby provided an XPO-1 inhibitor for use in the treatment and/or prevention of a hematologic neoplasm in a patient wherein the treatment further comprises administration of a hematopoietic cell transplant (e.g. allogenic, e.g. comprising T cells) and an MDM2 inhibitor.

### DETAILED DESCRIPTION OF THE INVENTION

All cited documents of the patent and non-patent literature are hereby incorporated by reference in their entirety.

The invention therefore relates to a mouse double minute 2 (MDM2) inhibitor for use in the treatment and/or prevention of a hematologic neoplasm relapse after hematopoietic cell transplantation (HCT) in a patient.

As used herein “prevention” of a hematologic neoplasm relapse is understood as relating to any method, process or action that is directed towards ensuring that a hematologic neoplasm relapse will not occur. Prevention relates to a prophylactic treatment intended to avoid a situation of a relapse. A “prophylactic” treatment is a treatment administered to a subject who does not exhibit signs of a disease or exhibits only early signs for the purpose of decreasing the risk of developing pathology, in the present case the occurrence of a relapse after HCT.

The term “treatment” refers to a therapeutic intervention that ameliorates a sign or symptom of a disease or pathological condition (here relapse of a hematologic neoplasm after HCT) after it has begun to develop. As used herein, the term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease.

As used herein, the terms “subject” and “patient” includes both human and veterinary subjects, in particularly mammals, and other organisms. The term “recipient” relates to a patient or subject that receives HCT and the MDM2 inhibitor of the invention.

It is understood that the term “neoplasm” relates to new abnormal growth of tissue. Malignant neoplasms show a greater degree of anaplasia and have the properties of invasion and metastasis, compared to benign neoplasms. As used herein, the term “hematologic neoplasm” relates to neoplasms located in the blood and blood-forming tissue (the bone marrow and lymphatic tissue). The commonest forms are the various types of leukemia, of lymphoma, and myelodysplastic syndromes, in particular the progressive, life-threatening forms of myelodysplastic syndromes.

The term hematologic neoplasm comprises tumors and cancers of the hematopoietic and lymphoid tissues relating to tumors and cancers that affect the blood, bone marrow, lymph, and lymphatic system. Because the hematopoietic and lymphoid tissues are all intimately connected through both the circulatory system and the immune system, a disease affecting one will often affect the others as well, making myeloproliferation and lymphoproliferation (and thus the leukemias and the lymphomas) closely related and often overlapping problems.

Hematological malignancies that are subject of the present invention are malignant neoplasms (“cancers”), and they are generally treated by specialists in hematology and/or oncology, as a subspecialty of internal medicine, surgical and radiation oncologists are also concerned with such conditions. Hematological malignancies may derive from either of the two major blood cell lineages, myeloid and lymphoid cell lines. The myeloid cell line normally produces granulocytes, erythrocytes, thrombocytes, macrophages and mast cells; the lymphoid cell line produces B, T, NK and plasma cells. Lymphomas, lymphocytic leukemias, and myeloma are from the lymphoid line, while acute and chronic myelogenous leukemia, myelodysplastic syndromes and myeloproliferative diseases are myeloid in origin.

In the context of the present invention, leukemias include, but are not limited to acute non-lymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

According to the present invention, lymphomas include Hodgkin and non-Hodgkin lymphoma (B-cell and T-cell lymphoma) including, but not limited to diffuse large B-cell lymphoma (DLBCL), primary mediastinal B-cell lymphoma, follicular lymphoma, chronic lymphocytic leukemia, small lymphocytic lymphoma, Mantle cell lymphoma, Marginal zone B-cell lymphomas, Extranodal marginal zone B-cell lymphomas, also known as mucosa-associated lymphoid tissue (MALT) lymphomas, nodal marginal zone B-cell lymphoma and splenic marginal zone B-cell lymphoma, Burkitt lymphoma, lymphoplasmacytic lymphoma (Waldenstrom macroglobulinemia), hairy cell leukemia primary central nervous system (CNS) lymphoma, precursor T-lymphoblastic lymphoma/leukemia, peripheral T-cell lymphomas, cutaneous T-cell lymphomas (mycosis fungoides, Sezary syndrome, and others), adult T-cell leukemia/lymphoma including the smoldering, the chronic, the acute and the lymphoma subtype, angioimmunoblastic T-cell lymphoma, extranodal natural killer/T-cell lymphoma, nasal type, enteropathy-associated intestinal T-cell lymphoma (EATL), anaplastic large cell lymphoma (ALCL), and unspecified peripheral T-cell lymphoma.

Myelodysplastic syndromes (MDS) are a group of cancers in which immature blood cells in the bone marrow do not mature, so do not become healthy blood cells. Symptoms may include feeling tired, shortness of breath, easy bleeding, or frequent infections. Some types may develop into acute myeloid leukemia.

Acute myeloid leukemia (AML) is a cancer of the myeloid line of blood cells, characterized by the rapid growth of abnormal cells that build up in the bone marrow and blood and interfere with normal blood cell production. Symptoms may include feeling tired, shortness of breath, easy bruising and bleeding, and increased risk of infection. Occasionally, spread may occur to the brain, skin, or gums. As an acute leukemia, AML progresses rapidly and is typically fatal within weeks or months if left untreated. AML typically is initially treated with chemotherapy, with the aim of inducing remission. People may then go on to receive additional chemotherapy, radiation therapy, or a stem cell transplant. The specific genetic mutations present within the cancer cells may guide therapy, as well as determine how long that person is likely to survive.

Aggressive forms of hematologic neoplasms and hematological malignancies require treatment with chemotherapy, radiotherapy, immunotherapy and a bone marrow transplant, which is a form of hematopoietic cell transplantation (HCT).

Hematopoietic cell transplantation (HCT) (also referred to as hematopoietic stem cell transplantation (HSCT)) is the transplantation of multipotent hematopoietic stem cells, usually derived from bone marrow, peripheral blood, or umbilical cord blood. HCT may be autologous (the patient's own stem cells are used), allogeneic (the stem cells come from a donor) or syngeneic (from an identical twin). HCT is performed for patients with certain cancers of the blood or bone marrow or lymphatic system, such as multiple myeloma or leukemia. In these cases, the recipient's immune system is usually fully (or in some cases only partially) destroyed with radiation and/or chemotherapy or other methods known in the art before the transplantation of hematopoietic stem cell grafts (myeloablation or partial mayeloablation). Infection and graft-versus-host disease are major complications of allogeneic HCT. HCT is a dangerous procedure with many possible complications and is therefore almost exclusively performed on patients with life-threatening diseases.

In the context of the present invention, it is preferred that the HCT is allogeneic. In comparison to autologous HCT the risk of cancer recurrence/relapse is reduced. Allogeneic HCT involves a (healthy) donor and a (patient) recipient. Allogeneic HCT donors must have a tissue type (human leukocyte antigen, HLA) that matches that of the recipient. Matching is usually performed based on variability at three or more loci of the HLA gene, and a perfect match at these loci is preferred. Even if there is a good match at these critical alleles, the recipient will require immunosuppressive medications to mitigate graft-versus-host disease. Allogeneic transplant donors may be related (usually a closely HLA matched sibling) or unrelated (donor who is not related and found to have very close degree of HLA matching). Allogeneic transplants are also performed using umbilical cord blood as the source of stem cells. In general, by transfusing healthy stem cells to the recipient's bloodstream to reform a healthy immune system, allogeneic HCT appear to improve chances for cure or long-term remission once the immediate transplant-related complications are resolved.

A compatible donor is found by doing additional HLA-testing from the blood of potential donors. The HLA genes fall in two categories (Type I and Type II). In general, mismatches of the Type-I genes (i.e. HLA-A, HLA-B, or HLA-C) increase the risk of graft rejection. A mismatch of an HLA Type II gene (i.e. HLA-DR, or HLA-DQB1) increases the risk of graft-versus-host disease.

Possible sources of donor cells include bone marrow, peripheral blood stem cells, amniotic fluid and umbilical cord blood, without limitation.

Graft-versus-host disease (GVHD) is an inflammatory disease that is unique to allogeneic transplantation and which is mediated by an attack by the “new” bone marrow's immune cells against the recipient's tissues. This can occur even if the donor and recipient are HLA-identical because the immune system can still recognize other differences between their tissues. Acute graft-versus-host disease typically occurs in the first 3 months after transplantation and may involve the skin, intestine, or the liver. High-dose corticosteroids, such as prednisone, are a standard treatment; however, this immunosuppressive treatment often leads to deadly infections. Chronic graft-versus-host disease may also develop after allogeneic transplant and is the major source of late treatment-related complications, although it less often results in death.

In embodiments of the invention, transplanted allo-T cells mediate a graft-versus-tumor effect (GvT) that is enhanced by MDM2 inhibition as described herein. The GvT effect appears after allogeneic HCT. The graft can contain donor T cells (T lymphocytes) that can be beneficial for the recipient by eliminating residual malignant cells, and in the context of the invention it is possible that the patient received one or more additional allogeneic T-cell transplantation.

GvT might develop after recognizing tumor-specific or recipient-specific alloantigens. It can lead to remission or immune control of hematologic malignancies and can therefore be exploited in the context of prevention or treatment of hematologic neoplasm relapse after HCT. This effect applies in myeloma and lymphoid leukemias, lymphoma, multiple myeloma and possibly breast cancer and may be referred to as graft versus leukemia effect or graft versus lymphoma effect or graft versus multiple myeloma effect in the context of the present invention. It is closely linked with graft-versus-host disease (GvHD), as the underlying principle of alloimmunity is the same. CD4+CD25+ regulatory T cells (Treg) can be used to suppress GvHD without loss of beneficial GvT effect and a person skilled in the art is able to adjust specific embodiments of the invention in order to fine tune the GvT effect. GvT most likely involves the reaction with polymorphic minor histocompatibility antigens expressed either specifically on hematopoietic cells or more widely on a number of tissue cells or tumor-associated antigens. GvT is mediated largely by cytotoxic T lymphocytes (CTL) but it can be employed by natural killers (NK cells) as separate effectors.

Graft-versus-leukemia (GvL) is a specific type of GvT effect and is a reaction against leukemic cells of the host that may remain and/or expand after myeloablative treatment before HCT leading to a relapse of the patient. GvL requires genetic disparity because the effect is dependent on the alloimunity principle and is a part of the reaction of the graft against the host. Whereas graft-versus-host-disease (GvHD) has a negative impact on the host, GvL is beneficial for patients with hematopoietic malignancies. After HCT both GvL and GvHD can develop. The interconnection of those two effects can be seen by comparison of leukemia relapse after HCT with development of GvHD. Patients who develop chronic or acute GvHD have lower chance of leukemia relapse. When transplanting T-cell depleted stem cell transplant, GvHD can be partially prevented, but in the same time the GvL effect is also reduced, because T-cells play an important role in both of those effects.

Accordingly, T-cell depletion is not preferred in the context of the present invention. The possibilities of GvL effect in the treatment of hematopoietic malignancies are limited by GvHD. The ability to induce GvL but not GvH after HCT would be very beneficial for those patients. There are some strategies to suppress the GvHD after transplantation or to enhance GvL but none of them provide an ideal solution to this problem. However, the use of MDM2 inhibitiors as described herein represents a new strategy enabling the promotion of GvL and GvT reactions.

For some forms of hematopoietic malignancies, for example acute myeloid leukemia (AML), the essential cells during HCT are, beside the donors T cells, the NK cells, which interact with KIR receptors. NK cells are within the first cells to repopulate host's bone marrow which means they play important role in the transplant engraftment. For their role in the GvL effect, their alloreactivity is required. Because KIR and HLA genes are inherited independently, the ideal donor can have compatible HLA genes and KIR receptors that induce the alloreaction of NK cells at the same time. This will occur with most of the non-related donors.

When using non-depleted T-cell transplant, cyclophosphamide is used after transplantation to prevent GvHD or transplant rejection. Other strategies currently clinically used for suppressing GvHD and enhancing GvL are for example optimization of transplant condition or donor lymphocyte infusion (DLI) after transplantation. One of the possibilities is the use of cytokines. Granulocyte colony-stimulating factor (G-CSF) is used to mobilize HSC and mediate T cell tolerance during transplantation. G-CSF can help to enhance GvL effect and suppress GvHD by reducing levels of LPS and TNF-α. Using G-CSF also increases levels of Treg, which can also help with prevention of GvHD. Other cytokines can also be used to prevent or reduce GvHD without eliminating GvL, for example KGF, IL-11, IL-18 and IL-35.

Since allogeneic HCT represents an intensive curative treatment for high-risk malignancies, its failure to prevent relapse leaves few options for successful salvage treatment. While many patients have a high early mortality from relapse, some respond and have sustained remissions, and a minority has a second chance of cure with appropriate therapy. The present invention represents a new strategy for treating and preventing relapse after HCT, since MDM2 inhibition increases visibility of remaining or recurring cancer cells for allo-T cells. The prognosis for relapsed hematological malignancies after HCT mostly depends on four factors: the time elapsed from SCT to relapse (with relapses occurring within 6 months having the worst prognosis), the disease type (with chronic leukemias and some lymphomas having a second possibility of cure with further treatment), the disease burden and site of relapse (with better treatment success if disease is treated early), and the conditions of the first transplant (with superior outcome for patients where there is an opportunity to increase either the alloimmune effect, the specificity of the antileukemia effect with targeted agents or the intensity of the conditioning in a second transplant). These features direct treatments toward either modified second transplants, chemotherapy, targeted antileukemia therapy, immunotherapy or palliative care. Relapse after HCT is an important problem in oncology and a skilled person is aware of the current understanding of the pathomechanisms leading to relapse, current treatment options and patient management in case of relapse after HCT, as reviewed for example by Barrett et al. (Expert Rev Hematol. 2010 August; 3(4): 429-441.doi: 10.1586/ehm.10.32).

Mouse double minute 2 homolog (MDM2) is also known as E3 ubiquitin-protein ligase Mdm2 and is a protein that in humans is encoded by the MDM2 gene. MDM2 is an important negative regulator of the p53 tumor suppressor and functions both as an E3 ubiquitin ligase that recognizes the N-terminal trans-activation domain (TAD) of the p53 tumor suppressor and as an inhibitor of p53 transcriptional activation.

MDM2 is also required for organ development and tissue homeostasis because unopposed p53 activation leads to p53-overactivation-dependent cell death, referred to as podoptosis. Podoptosis is caspase-independent and, therefore, different from apoptosis. The mitogenic role of MDM2 is also needed for wound healing upon tissue injury, while MDM2 inhibition impairs re-epithelialization upon epithelial damage. In addition, MDM2 has p53-independent transcription factor-like effects in nuclear factor-kappa beta (NFκB) activation. Therefore, MDM2 promotes tissue inflammation and MDM2 inhibition has potent anti-inflammatory effects in tissue injury. So, MDM2 blockade had mostly anti-inflammatory and anti-mitotic effects that can be of additive therapeutic efficacy in inflammatory and hyperproliferative disorders such as certain cancers or lymphoproliferative autoimmunity, such as systemic lupus erythematosus or crescentic glomerulonephritis. The key target of Mdm2 is the p53 tumor suppressor. Mdm2 has been identified as a p53 interacting protein that represses p53 transcriptional activity. Mdm2 achieves this repression by binding to and blocking the N-terminal trans-activation domain of p53. Mdm2 is a p53 responsive gene—that is, its transcription can be activated by p53. Thus, when p53 is stabilized, the transcription of Mdm2 is also induced, resulting in higher Mdm2 protein levels. The function of MDM2 and its role in cancer is a subject of extensive research and has been review in the art, for example by Li et al. (Front. Pharmacol., 7 May 2020, volume 11, Article 631, “Targeting Mouse Double Minute 2: Current Concepts in DNA Damage Repair and Therapeutic Approaches in Cancer”). The same article also reviews MDM2 inhibitors that are currently under clinical investigation for the treatment of various cancers. The use of the inhibitors discussed in this publication for the treatment and/or prevention of relapse of hematologic neoplasms after HCT is comprised by the present invention.

The functions of MDM2 have identified MDM2 as a promising target for the design of inhibitors to be used as anti-cancer drugs. Considering the deficiency of single target drugs in therapeutic effect maintenance over time as well as the conduciveness to activate alternative signaling pathways facilitating drug resistance, dual or multi-targeting MDM2 inhibitors are emerging. Many different MDM2 inhibitors have already been successfully developed for the clinical trials so that a person skilled in the art is well aware of the meaning of the term “MDM2 inhibitor” and also can easily identify multiple examples of such inhibitors known in the art. These include, for example, RG7112 (R05045337), idasanutlin (RG7388), AMG-232 (KRT-232), APG-115, BI-907828, CGM097, siremadlin (HDM-201), and milademetan (DS-3032b).

Nutlins are a series of cis-imidazoline analogs identified to bind MDM2 in the p53-binding pocket, leading to cell cycle arrest and apoptosis in cancer cells, as well as growth inhibition of human tumor xenografts in nude mice. Several inhibitors targeting MDM2-p53 such as RG7112, RG7388, RG7775, SAR405838, HDM201, APG-115, AMG-232, and MK-8242 have recently been developed to treat human cancers with clinical trials.

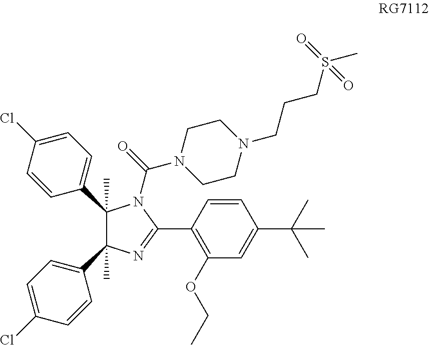 [SMILES: CCOc1cc(C(C)(C)C)ccc1C1=N[C@@](C)(c2ccc(Cl)cc2)[C@@](C)(c2ccc(Cl)cc2)N1C(=O)N1CCN(CCCS(C)(=O)=O)CC1]

was the first small-molecule MDM2 inhibitor to enter human clinical trials and which was derived from structural modification of Nutlin-3a. RG7112 was designed to target MDM2 in p53-binding pocket and restored p53 activity inducing robust p21 expression and apoptosis in p53 wild-type glioblastomas cell. So far, seven clinical studies on RG7112 have been completed (http://www.clinicaltrials.gov/; NCT01677780, NCT01605526, NCT01143740, NCT01164033, NCT00559533, NCT00623870, NCT01677780). Study of NP25299 (NCT01164033) was an open-label, randomized, cross-over study in patients with solid tumors. It evaluated the effects of food on the pharmacokinetics of single oral doses of RG7112. This study included two parts: the first one comprised an initial single-dose, while the other comprised four different treatment schedules of increased doses. The results indicated that RG7112 was generally well tolerated with GI toxicities, the most common AEs, making it treatable with anti-emetics (Patnaik et al., 2015).

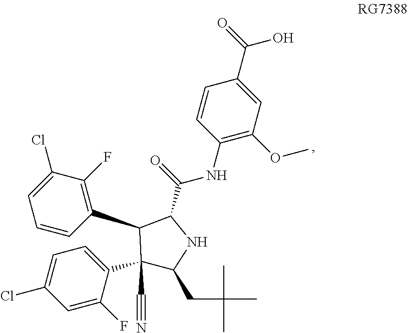 [SMILES: COc1cc(C(=O)O)ccc1NC(=O)[C@@H]1N[C@@H](CC(C)(C)C)[C@](C#N)(c2ccc(Cl)cc2F)[C@H]1c1cccc(Cl)c1F]

a second-generation Nutlin, was developed to improve the potency and toxicity profile of earlier Nutlin. RG7388 induced p21 expression and effective cell cycle arrest in three cell lines MCF-7, U-20S and SJSA-1, which proved the strong activation of p53. RG7388 is currently undergoing several clinical examinations, including the only III clinical trial of MDM2 inhibitor (MIRROS/NCT02545283). The results of phase I clinical trial showed that RG7388 improved clinical outcomes by modulating p53 activity in AML patients with high levels of MDM2 expression. MIRROS is a randomized phase III clinical trial to evaluate the efficacy of RG7388 combined with cytarabine in the treatment of recurrent and refractory acute myeloid leukemia (AML). As of April 2019, the study has recruited approximately 90% of patient population and is still ongoing. If 80% of deaths are observed in p53-WT population of this study, an interim efficacy analysis can be obtained by 2020. MIRROS may obtain the first phase III clinical trial data of MDM2 inhibitors and provide a new treatment option for patients with AML.

RG7775 is an inactive pegylated prodrug of AP (idasanutlin), which cleaves the pegylated tail of esterases in the blood. AP is a potent and selective inhibitor of p53-MDM2 interaction to activate p53 pathway and associates with cell-cycle arrest and/or apoptosis. In a preclinical trial, intravenous (IV) RG7775 (R06839921) showed anti-tumor effects in osteosarcoma and AML in immunocompromised mice model. In a phase I study (NCT02098967), RG7775 was investigated for its safety, tolerability, and pharmacokinetics in patients with advanced malignancies. The result showed that RG7775 had a safety profile comparable to oral idasanutlin.

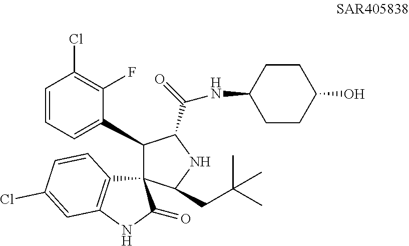 [SMILES: CC(C)(C)C[C@@H]1N[C@@H](C(=O)N[C@H]2CC[C@H](O)CC2)[C@H](c2cccc(Cl)c2F)[C@]12C(=O)Nc1cc(Cl)ccc12]

is an oral selective spirooxindole small molecule derivative antagonist of MDM2, which targets MDM2-p53 interaction. In the treatment of dedifferentiated liposarcoma cells, SAR405838 effectively stabilized p53, activated p53 pathway, block cell proliferation, promoted cell-cycle arrest and induced apoptosis. SAR405838 has been used in two clinical trials in cancer patients (NCT01636479, NCT01985191). Study of TED12318 (NCT01636479) was a phase I, open-label, dose-ranging, dose escalating, safety study administered orally in adult patients with advanced solid tumor. In this trial, 74 patients were treated with SAR405838 which showed best response in 56% patients with a 32% 3-month progression free rate. This study indicated that SAR405838 had an acceptable safety profile in patients with advanced solid tumors. Another clinical trial on SAR405838 was the study of TCD13388 (NCT01985191), which analyzed safety and efficacy of SAR405838 combined with pimasertib in cancer patients. In this study, 26 patients with locally advanced or metastatic solid tumors, who were documented to have wild-type p53 and RAS or RAF mutations, were enrolled in this study. The aim of this study was to explore maximum tolerated dose (MTD). Patient response was observed with SAR405838 at 200 or 300 mg QD plus pimasertib 60 mg QD or 45 mg BID. The most frequently occurring adverse events observed were diarrhoea (81%), blood creatine phosphokinase (77%), nausea (62%) and vomiting (62%). This study indicated that the safety profile of SAR405838 combined with pimasertib was consistent with the safety profiles of both the drugs.

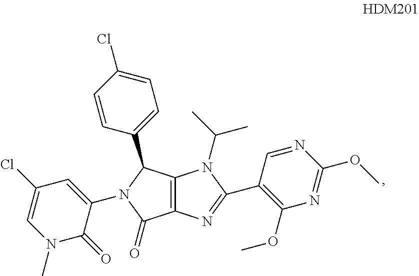 [SMILES: COc1ncc(-c2nc3c(n2C(C)C)[C@H](c2ccc(Cl)cc2)N(c2cc(Cl)cn(C)c2=O)C3=O)c(OC)n1]

also called siremadlin or NVP-HDM201, is a potent and selective small molecule that inhibits the interaction between MDM2 and p53, leading to tumor regression in preclinical models with both low and high dose regimen. The compound and related compounds of similar activity have been extensively described in WO2013/111105A1 as well as in WO2019/073435A1. HDM201 had a specific and effective killing effect on p53 wild-type cells with positive-ITD when used in combination with midotaline. HDM201 has been used in clinal trial (NCT02143635). NCT02143635 determined and evaluated a safe and tolerated dose of HDM201 in patients with advanced tumors with wild type p53. At the time of data cut-off (Apr. 1, 2016), 74 patients received HDM201 (Reg 1 with 38 patients and Reg 2 with 36 patients still receiving treatment). The results showed that the common grade 3/4 adverse events (AEs) in both regimens (Reg 1 and Reg 2) were anemia (8%; 17%), neutropenia (26%; 14%), and thrombocytopenia (24%; 28%). Preliminary data indicated that hematological toxicity was delayed and dependent on regimen and that the Reg 1 regimen allows for higher cumulative dose.

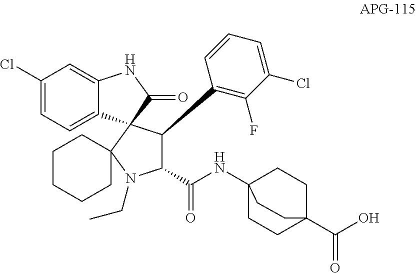 [SMILES: CCN1[C@@H](C(=O)NC23CCC(C(=O)O)(CC2)CC3)[C@H](c2cccc(Cl)c2F)[C@]2(C(=O)Nc3cc(Cl)ccc32)C12CCCCC2]

is a novel, orally active small-molecule MDM2 inhibitor. APG-115 restores p53 expression after binding with MDM2 and activates p53 mediated apoptosis in tumor cells with wild-type p53. APG-115 has been used in clinical trials for treating solid tumor (NCT02935907), metastatic melanoma (NCT03611868), and salivary gland carcinoma (NCT03781986). Study NCT02935907 was a phase I study of the safety, pharmacokinetic and pharmacodynamic properties of orally administered APG-115 in patients with advanced solid tumors or lymphomas. Different dose levels (Including 10 mg, 20 mg, 50 mg, 100 mg, 200 mg and 300 mg) were tested in this study. The result showed the optimum dose of APG-115 to be 100 mg with no dose-limiting toxicities. In recent studies, APG-115 mediated the anti-tumor immunity of tumor microenvironment (TME). APG-115 activated p53 and p21 on bone marrow-derived macrophages in vitro, and reduced the number of immunosuppressive M2 macrophages by down-regulating c-Myc and c-Maf. In addition, APG-115 showed costimulatory activity in T cells and increased the expression of PD-L1 in tumor cells. This evidence suggests the combination of APG and immunotherapy may be a new anti-tumor regimen.

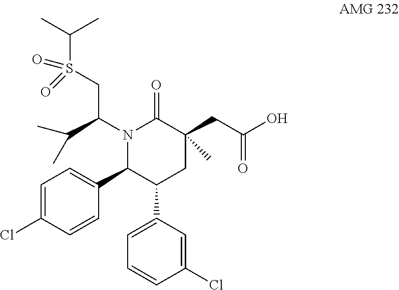 [SMILES: CC(C)[C@@H](CS(=O)(=O)C(C)C)N1C(=O)[C@@](C)(CC(=O)O)C[C@H](c2cccc(Cl)c2)[C@H]1c1ccc(Cl)cc1]

is an investigational oral, selective MDM2 inhibitor that restores p53 tumor suppression by blocking MDM2-p53 interaction. The activity of AMG 232 and its effect on p53 signal were characterized in several preclinical tumor models. AMG 232 bind MDM2, strongly induced p53 activity, lead to cell cycle arrest and inhibit tumor cell proliferation. Several clinical trials of the AMG 232 such as NCT01723020, NCT02016729, NCT02110355, NCT03031730, NCT03041688, NCT03107780, and NCT03217266 have been ongoing to treat human cancers. NCT02016729 was an open-label phase I study that evaluated the safety, pharmacokinetics, and MTD of AMG 232. In this study, AMG 232 was administered in two regimens (arm 1 and arm 2). Patients were treated with AMG 232 at 60, 120, 240, 360, 480, or 960 mg as monotherapy once daily for 7 d every 2 weeks in arm 1 or at 60 mg combined with trametinib at 2 mg in arm 2. The results exhibited common treatment-related AEs included nausea (58%), diarrhea (56%), vomiting (33%), and decreased appetite (25%). However, the MTD of AMG 232 was not reached. Dose escalation was discontinued because of its unacceptable gastrointestinal AEs at higher doses.

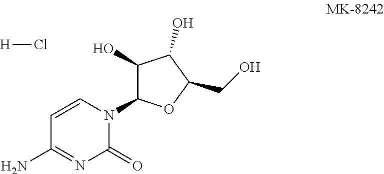 [SMILES: Cl ; Nc1ccn([C@@H]2O[C@H](CO)[C@@H](O)[C@@H]2O)c(=O)n1]

is a potent, small-molecule inhibitor which targets MDM2-p53 interaction. MK-8242 induced tumor regression of various solid tumor types and complete or partial response in most acute lymphoblastic leukemia xenografts. MK-8242 has been used in two Phase I clinical trials (NCT01451437 and NCT01463696). Study of NCT01451437 was a study of MK-8242 alone and in combination with cytarabine in adult participants with refractory or recurrent AML. In this study MK-8242 was administered at 30-250 mg (p.o;QD) or 120-250 mg (p.o;BID) for 7 d on/7 d off in a 28-d cycle and optimized regimen was administered at 210 or 300 mg (p.o;BID) for 7 on/14 off in 21-d cycle. Twenty-six patients were enrolled in this study, out of which 5 discontinued because of AEs and 7 patients died. This study showed the 7 on/14 off regimen had a more favorable safety profile than the 7 on/7 off regimen. NCT01463696 was aimed at evaluating the safety and pharmacokinetic profile of MK-8242 in patients with advanced solid tumors. In this study, drug dose was escalated to determine the MTD in part 1 and the MTD was confirmed and the recommended Phase 2 dose (RPTD) was established in part 2. Finally, 47 patients were enrolled in this study and treated with MK-8242 at eight level doses that ranged from 60 to 500 mg. The result showed that MK-8242 activated p53 pathway with an acceptable tolerability profile at 400 mg (BID).

MDM2 inhibitor BI 907828 is an orally available inhibitor of murine double minute 2 (MDM2), with potential antineoplastic activity. Upon oral administration, BI 907828 binds to MDM2 protein and prevents its binding to the transcriptional activation domain of the tumor suppressor protein p53. By preventing MDM2-p53 interaction, the transcriptional activity of p53 is restored. This leads to p53-mediated induction of tumor cell apoptosis. Compared to currently available MDM2 inhibitors, the pharmacokinetic properties of BI 907828 allow for more optimal dosing and dose schedules that may reduce myelosuppression, an on-target, dose-limiting toxicity for this class of inhibitors.

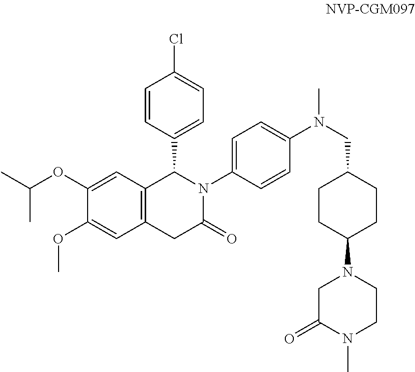 [SMILES: COc1cc2c(cc1OC(C)C)[C@H](c1ccc(Cl)cc1)N(c1ccc(N(C)C[C@H]3CC[C@H](N4CCN(C)C(=O)C4)CC3)cc1)C(=O)C2]

is a highly potent and selective MDM2 inhibitor with Ki value of 1.3 nM for hMDM2 in TR-FRET assay. It binds to the p53 binding-site of the Mdm2 protein, disrupting the interaction between both proteins, leading to an activation of the p53 pathway.

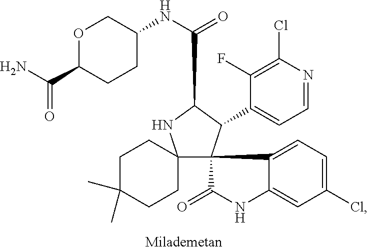 [SMILES: CC1(C)CCC2(CC1)N[C@@H](C(=O)N[C@@H]1CC[C@@H](C(N)=O)OC1)[C@H](c1ccnc(Cl)c1F)[C@]21C(=O)Nc2cc(Cl)ccc21]

is an orally available MDM2 (murine double minute 2) antagonist with potential antineoplastic activity. Upon oral administration, milademetan binds to, and prevents the binding of MDM2 protein to the transcriptional activation domain of the tumor suppressor protein p53. By preventing this MDM2-p53 interaction, the proteasome-mediated enzymatic degradation of p53 is inhibited and the transcriptional activity of p53 is restored. This results in the restoration of p53 signaling and leads to the p53-mediated induction of tumor cell apoptosis. MDM2, a zinc finger protein and a negative regulator of the p53 pathway, is overexpressed in cancer cells; it has been implicated in cancer cell proliferation and survival.

Salts of any of the above compounds are also within the scope of the invention.

As used herein, an MDM2 inhibitor can be a compound as disclosed in U.S. application Ser. No. 11/626,324, published as US Application Publication No. 2008/0015194; U.S. Nonprovisional application Ser. No. 12/986,146; International Application No. PCT/US11/20414, published as WO 2011/085126; or International Application No. PCT/US11/20418, published as WO 2011/085129; each of which is incorporated herein by reference.

An MDM2 inhibitor can be a compound as disclosed in Vassilev 2006 Trends in Molecular Medicine 13(1), 23-31. For example, an MDM2 inhibitor can be a nutlin (e.g., a cis-imidazole compound, such as nutlin-3a); a benzodiazepine as disclosed in Grasberger et al. 2005 J Med Chem 48, 909-912; a RITA compound as disclosed in Issaeva et al. 2004 Nat Med 10, 1321-1328; a spiro-oxindole compound as disclosed in Ding et al. 2005 J Am Chem Soc 127, 10130-10131 and Ding et al. 2006 J Med Chem 49, 3432-3435; or a quininol compound as disclosed in Lu et al. 2006 J Med Chem 49, 3759-3762. As a further example, an MDM2 inhibitor can be a compound as disclosed in Chene 2003 Nat. Rev. Cancer 3, 102-109; Fotouhi and Graves 2005 Curr Top Med Chem 5, 159-165; or Vassilev 2005 J Med Chem 48, 4491-4499.

It is an important advantage of the MDM2 inhibitors of the invention that MDM2-inhibition promotes cytotoxicity and longevity of donor T cells.

In embodiments, MDM2 inhibition can influence the phenotype of the allo-T cells in the patient, leading to increased cytotoxicity and longevity. For example, MDM2 inhibition can cause allo-T cells to upregulate expression of Bcl-2-receptor and 1L7-receptor (DE127), markers that are associated with longevity. Furthermore, upregulated expression of cytotoxicity markers, such as increases expression of perforin, CD107a, IFN-γ, TNF and CD69 by CD8+ allo-T cells can be observed upon MDM2 inhibition by an MDM2 inhibitor in the context of the present invention.

A cytotoxic T cell (also known as cytotoxic T lymphocyte, CTL, T-killer cell, cytolytic T cell, CD8+ T-cell or killer T cell) is a T lymphocyte (a type of white blood cell) that kills cancer cells, cells that are infected (particularly with viruses), or cells that are damaged in other ways. Most cytotoxic T cells express T-cell receptors (TCRs) that can recognize a specific antigen. An antigen is a molecule capable of stimulating an immune response and is often produced by cancer cells or viruses. Antigens inside a cell are bound to class I MHC molecules, and brought to the surface of the cell by the class I MHC molecule, where they can be recognized by the T cell. If the TCR is specific for that antigen, it binds to the complex of the class I MHC molecule and the antigen, and the T cell destroys the cell. In order for the TCR to bind to the class I MHC molecule, the former must be accompanied by a glycoprotein called CD8, which binds to the constant portion of the class I MHC molecule. Therefore, these T cells are called CD8+ T cells. The affinity between CD8 and the MHC molecule keeps the TC cell and the target cell bound closely together during antigen-specific activation. CD8+ T cells are recognized as TC cells once they become activated and are generally classified as having a pre-defined cytotoxic role within the immune system. CD8+ T cells also can make some cytokines.

Administration of an MDM2 inhibitor can induce upregulation and increased expression of TNF-related apoptosis-inducing ligand receptor 1(TRAIL-R1), TRAIL-R2, human leukocyte antigen (HLA) class I molecules and HLA class II molecules on cancer cells of the patient. TNF-related apoptosis-inducing ligand (TRAIL), is a protein functioning as a ligand that induces the process of cell death called apoptosis. TRAIL is a cytokine that is produced and secreted by most normal tissue cells. It causes apoptosis primarily in tumor cells, by binding to certain death receptors, TRAIL-R1 or TRAIL-R2. TRAIL has also been designated CD253 (cluster of differentiation 253) and TNFSF10 (tumor necrosis factor (ligand) superfamily, member 10.

The TNF-related apoptosis-inducing ligand (TRAIL) and its five cellular receptors constitute one of the three death-receptor/ligand systems that have been shown to regulate intercellular apoptotic responses in the immune system. In different systems of antigenic or tumor challenge, the TRAIL/TRAIL receptor system was shown to have immunosuppressive, immunoregulatory, proviral or antiviral, and tumor immunosurveillance functions. TRAIL can bind two apoptosis-inducing receptors—TRAIL-R1 (DR4) and TRAIL-R2 (DR5)—and two additional cell-bound receptors incapable of transmitting an apoptotic signal—TRAIL-R3 (LIT, DcR1) and TRAIL-R4 (TRUNDD, DcR2)—sometimes called decoy receptors. The initial step of apoptosis induction by TRAIL is the binding of the ligand to TRAIL-R1 or TRAIL-R2. Thereby the receptors are trimerized and the death-inducing signaling complex (DISC) is assembled. The adaptor molecule, Fas-associated death domain (FADD), translocates to the DISC where it interacts with the intracellular death domain (DD) of the receptors. Via its second functional domain, the death effector domain (DED), FADD recruits procaspases 8 and 10 to the DISC where they are autocatalytically activated. This activation marks the start of a caspase-dependent signaling cascade. Full activation of effector caspases leads to cleavage of target proteins, fragmentation of DNA and, ultimately, to cell death. The function of TRAIL and TRAIL-R1 and TRAIL-R2 have been described in the art, for example by Falschlehner et al. (Immunology. 2009 June; 127(2): 145-154).

It was surprisingly found that administration MDM2 inhibition enhances TRAIL-R1/R2 expression on cancer cells in the context of the invention which was at least partially required for mediating the cytotoxic effect of allo-T cells in the context of the invention since absence of TRAIL on the T cells resulted in strongly reduced killing.

Furthermore, it was completely unexpected that MDM2-inhibition could upregulate MHC proteins on cancer cells, such as leukemic cells and in particular AML cells, thereby enhancing their vulnerability to allogeneic T cells after HCT and allo-T cell transplantation.

The major histocompatibility complex (MHC) is a large locus on vertebrate DNA containing a set of closely linked polymorphic genes that code for cell surface proteins essential for the adaptive immune system. This locus got its name because it was discovered in the study of tissue compatibility upon transplantation. Later studies revealed that tissue rejection due to incompatibility is an experimental artifact masking the real function of MHC molecules—binding an antigen derived from self-proteins or from pathogen and the antigen presentation on the cell surface for recognition by the appropriate T-cells. MHC molecules mediate interactions of leukocytes, with other leukocytes or with body cells. The MHC determines compatibility of donors for organ transplant, as well as one's susceptibility to an autoimmune disease via cross-reacting immunization.

MHC class I molecules are expressed in all nucleated cells and also in platelets—in essence all cells but red blood cells. It presents epitopes to killer T cells, also called cytotoxic T lymphocytes (CTLs). A CTL expresses CD8 receptors, in addition to T-cell receptors (TCR)s. When a CTL's CD8 receptor docks to a MHC class I molecule, if the CTL's TCR fits the epitope within the MHC class I molecule, the CTL triggers the cell to undergo programmed cell death by apoptosis. Thus, MHC class I helps mediate cellular immunity, a primary means to address intracellular pathogens, such as viruses and some bacteria, including bacterial L forms, bacterial genus Mycoplasma, and bacterial genus Rickettsia. In humans, MHC class I comprises HLA-A, HLA-B, and HLA-C molecules.

MHC class II can be conditionally expressed by all cell types, but normally occurs only on “professional” antigen-presenting cells (APCs): macrophages, B cells, and especially dendritic cells (DCs). An APC takes up an antigenic protein, performs antigen processing, and returns a molecular fraction of it—a fraction termed the epitope—and displays it on the APC's surface coupled within an MHC class II molecule (antigen presentation). On the cell's surface, the epitope can be recognized by immunologic structures like T cell receptors (TCRs). The molecular region which binds to the epitope is the paratope. On surfaces of helper T cells are CD4 receptors, as well as TCRs. When a naive helper T cell's CD4 molecule docks to an APC's MHC class II molecule, its TCR can meet and bind the epitope coupled within the MHC class II. This event primes the naive T cell. According to the local milieu, that is, the balance of cytokines secreted by APCs in the microenvironment, the naive helper T cell (Th0) polarizes into either a memory Th cell or an effector Th cell of phenotype either type 1 (Th1), type 2 (Th2), type 17 (Th17), or regulatory/suppressor (Treg), as so far identified, the Th cell's terminal differentiation. MHC class II thus mediates immunization to—or, if APCs polarize Th0 cells principally to Treg cells, immune tolerance of—an antigen. The polarization during primary exposure to an antigen is key in determining a number of chronic diseases, such as inflammatory bowel diseases and asthma, by skewing the immune response that memory Th cells coordinate when their memory recall is triggered upon secondary exposure to similar antigens. B cells express MHC class II to present antigens to Th0, but when their B cell receptors bind matching epitopes, interactions which are not mediated by MHC, these activated B cells secrete soluble immunoglobulins: antibody molecules mediating humoral immunity. Class II MHC molecules are also heterodimers, genes for both α and β subunits are polymorphic and located within MHC class II subregion. Peptide-binding groove of MHC-II molecules is forms by N-terminal domains of both subunits of the heterodimer, α1 and β1, unlike MHC-I molecules, where two domains of the same chain are involved. In addition, both subunits of MHC-II contain transmembrane helix and immunoglobulin domains α2 or β2 that can be recognized by CD4 co-receptors. In this way MHC molecules chaperone which type of lymphocytes may bind to the given antigen with high affinity, since different lymphocytes express different T-Cell Receptor (TCR) co-receptors.

The human leukocyte antigen (HLA) system or complex is a group of related proteins that are encoded by the major histocompatibility complex (MHC) gene complex in humans. HLAs corresponding to MHC class I (A, B, and C), which all are the HLA Class1 group, present peptides from inside the cell. For example, if the cell is infected by a virus, the HLA system brings fragments of the virus to the surface of the cell so that the cell can be destroyed by the immune system. These peptides are produced from digested proteins that are broken down in the proteasomes. In general, these particular peptides are small polymers, of about 8-10 amino acids in length. Foreign antigens presented by MHC class I attract T-lymphocytes called killer T-cells (also referred to as CD8-positive or cytotoxic T-cells) that destroy cells. Some new work has proposed that antigens longer than 10 amino acids, 11-14 amino acids, can be presented on MHC I eliciting a cytotoxic T cell response.[3] MHC class I proteins associate with β2-microglobulin, which unlike the HLA proteins is encoded by a gene on chromosome 15.

HLAs corresponding to MHC class II (DP, DM, DO, DQ, and DR) present antigens from outside of the cell to T-lymphocytes. These particular antigens stimulate the multiplication of T-helper cells (also called CD4-positive T cells), which in turn stimulate antibody-producing B-cells to produce antibodies to that specific antigen. Self-antigens are suppressed by regulatory T cells.

Exportin 1 (XPO1), also known as chromosomal maintenance 1 (CRM1), is a eukaryotic protein that mediates the nuclear export of proteins, rRNA, snRNA, and some mRNA. Exportin 1 mediates leucine-rich nuclear export signal (NES)-dependent protein transport and specifically mediates the nuclear export of Rev and U snRNAs. It is involved in the control of several cellular processes by controlling the localization of cyclin B, MAPK, and MAPKAP kinase 2, and it also regulates NFAT and AP-1. Furthermore, it has been shown to interact with p53 and to mediate its export from the nucleus, thereby reducing expression of genes that are under p53 control, such as the genes encoding TRAIL-R1 and -R2 as well as MHC-II.

XPO1 is also upregulated in many malignancies and associated with a poor prognosis. Its inhibition has been a target of therapy, and hence, the selective inhibitors of nuclear transport (SINE) compounds were developed as a novel class of anti-cancer agents. The most well-known SINE agent is selinexor (KPT-330) and has been widely tested in phase I and II clinical trials in both solid tumors and hematologic malignancies.

Selective inhibitors of nuclear export (SINEs or SINE compounds) are drugs that block exportin 1 (XPO1 or CRM1), a protein involved in transport from the cell nucleus to the cytoplasm. This causes cell cycle arrest and cell death by apoptosis. Thus, SINE compounds are of interest as anticancer drugs; several are in development, and one (selinexor) has been approved for treatment of multiple myeloma as a drug of last resort. The prototypical nuclear export inhibitor is leptomycin B, a natural product and secondary metabolite of Streptomyces bacteria. SINEs include besides KPT-330 also for example KPT-8602, KPT-185, KPT-276 KPT-127, KPT- 205, and KPT-227. XPO-1 inhibition for therapeutic purposes has been reviewed in the literature, for example by Parikh et al (J Hematol Oncol. 2014; 7: 78).

As used herein, pharmaceutical compositions for administration to a subject can include at least one further pharmaceutically acceptable additive such as carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. The pharmaceutically acceptable carriers useful for these formulations are conventional. Remington's Pharmaceutical Sciences, by E. W. Martin, Mack Publishing Co., Easton, PA, 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In accordance with the various treatment methods of the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

“Administration of” and “administering a” compound or product should be understood to mean providing a compound, a prodrug of a compound, or a pharmaceutical composition as described herein. The compound or composition can be administered by another person to the subject (e.g., intravenously) or it can be self-administered by the subject (e.g., tablets).

Any references herein to a compound for use as a medicament in the treatment of a medical condition also relate to a method of treating said medical condition comprising the administration of a compound, or composition comprising said compound, to a subject in need thereof, or to the use of a compound, composition comprising said compound, in the treatment of said medical condition.

Dosage can be varied by the attending clinician to maintain a desired concentration at a target site (for example, the lungs, bone marrow or systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth.

The present invention also relates to a method of treatment of subjects as disclosed herein. The method of treatment comprises preferably the administration of a therapeutically effective amount of a compound and potentially further compounds or products disclosed herein to a subject in need thereof.

In the context of the present invention, the term “medicament” refers to a drug, a pharmaceutical drug or a medicinal product used to diagnose, cure, treat, or prevent disease. It refers to any substance or combination of substances presented as having properties for treating or preventing disease. The term comprises any substance or combination of substances, which may be used in or administered either with a view to restoring, correcting or modifying physiological functions by exerting a pharmacological, immunological or metabolic action, or to making a medical diagnosis. The term medicament comprises biological drugs, small molecule drugs or other physical material that affects physiological processes.

The MDM2 inhibitors and potentially further compounds according to the present invention as described herein may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, subcutaneously, subconjunctival, intravesicularly, mucosally, intrapericardially, intraumbilically, intraocularly, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

In the context of the present invention, the term “cancer therapy” refers to any kind of treatment of cancer, including, without limitation, surgery, chemotherapy, radiotherapy, irradiation therapy, hormonal therapy, targeted therapy, cellular therapy, cancer immunotherapy, monoclonal antibody therapy. The administration of MDM2 inhibitors as described herein can be embedded in a broader cancer therapy strategy.

Administration of the MDM2 inhibitor can be in combination with one or more other cancer therapies. In the context of the present invention the term “in combination” indicates that an individual that receives the compound according to the present invention also receives other cancer therapies, which does not necessarily happen simultaneously, combined in a single pharmacological composition or via the same route of administration. “In combination” therefore refers the treatment of an individual suffering from cancer with more than one cancer therapy. Combined administration encompasses simultaneous treatment, co-treatment or joint treatment, whereby treatment may occur within minutes of each other, in the same hour, on the same day, in the same week or in the same month as one another.

Cancer therapies in the sense of the present invention include but are not limited to irradiation therapy and chemotherapy and work by overwhelming the capacity of the cell to repair DNA damage, resulting in cell death.

In this context, chemotherapy refers to a category of cancer treatment that uses one or more anti-cancer drugs (chemotherapeutic agents) as part of a standardized chemotherapy regimen. Chemotherapy may be given with a curative intent (which almost always involves combinations of drugs), or it may aim to prolong life or to reduce symptoms (palliative chemotherapy). Chemotherapy is one of the major categories of medical oncology (the medical discipline specifically devoted to pharmacotherapy for cancer). Chemotherapeutic agents are used to treat cancer and are administered in regimens of one or more cycles, combining two or more agents over a period of days to weeks. Such agents are toxic to cells with high proliferative rates—e.g., to the cancer itself, but also to the GI tract (causing nausea and vomiting), bone marrow (causing various cytopenias) and hair (resulting in baldness).

Chemotherapeutic agents comprise, without limitation, Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, Vinorelbine.

Irradiation or radiation therapy or radiotherapy in the context of the present invention relates to a therapeutic approach using ionizing or ultraviolet-visible (UV/Vis) radiation, generally as part of cancer treatment to control or kill malignant cells such as cancer cells or tumor cells. Radiation therapy may be curative in a number of types of cancer, if they are localized to one area of the body. It may also be used as part of adjuvant therapy, to prevent tumor recurrence after surgery to remove a primary malignant tumor (for example, early stages of breast cancer). Radiation therapy is synergistic with chemotherapy, and can been used before, during, and after chemotherapy in susceptible cancers. Radiation therapy is commonly applied to the cancerous tumor because of its ability to control cell growth. Ionizing radiation works by damaging the DNA of cancerous tissue leading to cellular death. Radiation therapy can be used systemically or locally.

Radiation therapy works by damaging the DNA of cancerous cells. This DNA damage is caused by one of two types of energy, photon or charged particle. This damage is either direct or indirect ionization of the atoms which make up the DNA chain. Indirect ionization happens as a result of the ionization of water, leading to the formation of free radicals, including hydroxyl radicals, which then damage the DNA. In photon therapy, most of the radiation effect is mediated through free radicals. Cells have mechanisms for repairing single-strand DNA damage and double-stranded DNA damage. However, double-stranded DNA breaks are much more difficult to repair and can lead to dramatic chromosomal abnormalities and genetic deletions. Targeting double-stranded breaks increases the probability that cells will undergo cell death.

The amount of radiation used in photon radiation therapy is measured in gray (Gy) and varies depending on the type and stage of cancer being treated. For curative cases, the typical dose for a solid epithelial tumor ranges from 60 to 80 Gy, while lymphomas are treated with 20 to 40 Gy. Preventive (adjuvant) doses are typically around 45-60 Gy in 1.8-2 Gy fractions (for breast, head, and neck cancers.)

Different types of radiation therapy are known such as external beam radiation therapy, including conventional external beam radiation therapy, stereotactic radiation (radiosurgery), virtual simulation, 3-dimensional conformal radiation therapy, and intensity-modulated radiation therapy, intensity-modulated radiation therapy (IMRT), volumetric modulated arc therapy (VMAT), Particle therapy, auger therapy, brachytherapy, intraoperative radiotherapy, radioisotope therapy and deep inspiration breath-hold.

External beam radiation therapy comprises X-ray, gamma-ray and charged particles and can be applied as a low-dose rate or high dose rate depending on the overall therapeutic approach.

In internal radiation therapy radioactive substance can be bound to one or more monoclonal antibodies. For example, radioactive iodine can be used for thyroid malignancies. Brachytherapy of High dose regime (HDR) or low dose regime (LDR) can be combined with IR in prostate cancer.

According to the present invention, DNA damage-inducing chemotherapies comprise the administration of chemotherapeutics agents including, but not limited to anthracyclines such as Daunorubicin, Doxorubicin, Epirubicin, Idarubicin, Valrubicin, Mitoxantrone; Inhibitors of topoisomerase I such as Irinotecan (CPT-11) and Topotecan; Inhibitors of topoisomerase II including Etoposide, Teniposide and Tafluposide; Platinum-based agents such as Carboplatin, Cisplatin and Oxaliplatin; and other chemotherapies such as Bleomycin.

The instant disclosure also includes kits, packages and multi-container units containing the herein described pharmaceutical compositions, active ingredients, and/or means for administering the same for use in the prevention and treatment of diseases and other conditions in mammalian subjects.

### FIGURES

The invention is further described by the following figures. These are not intended to limit the scope of the invention but represent preferred embodiments of aspects of the invention provided for greater illustration of the invention described herein.

### Brief Description of the Figures

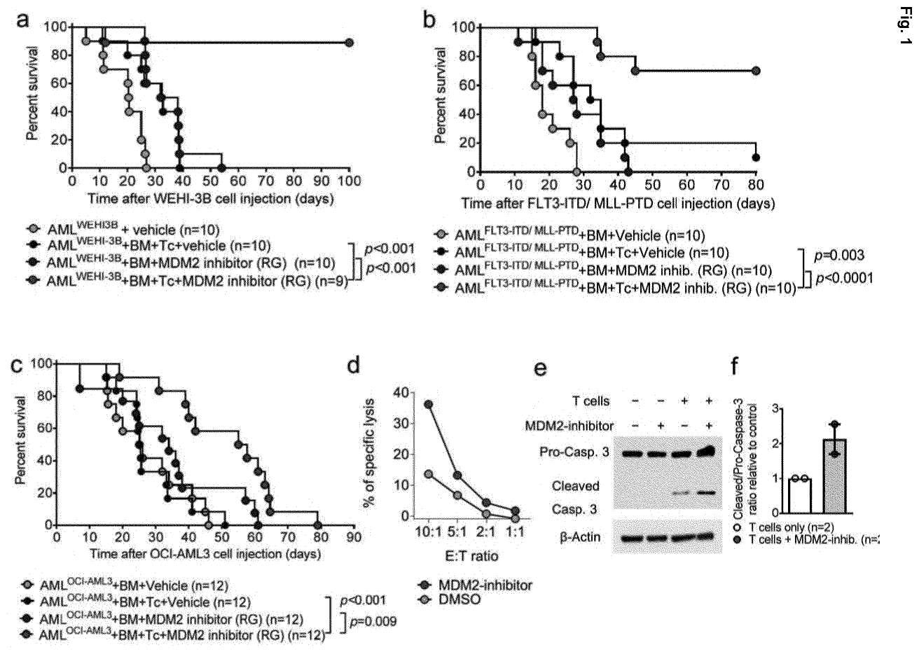

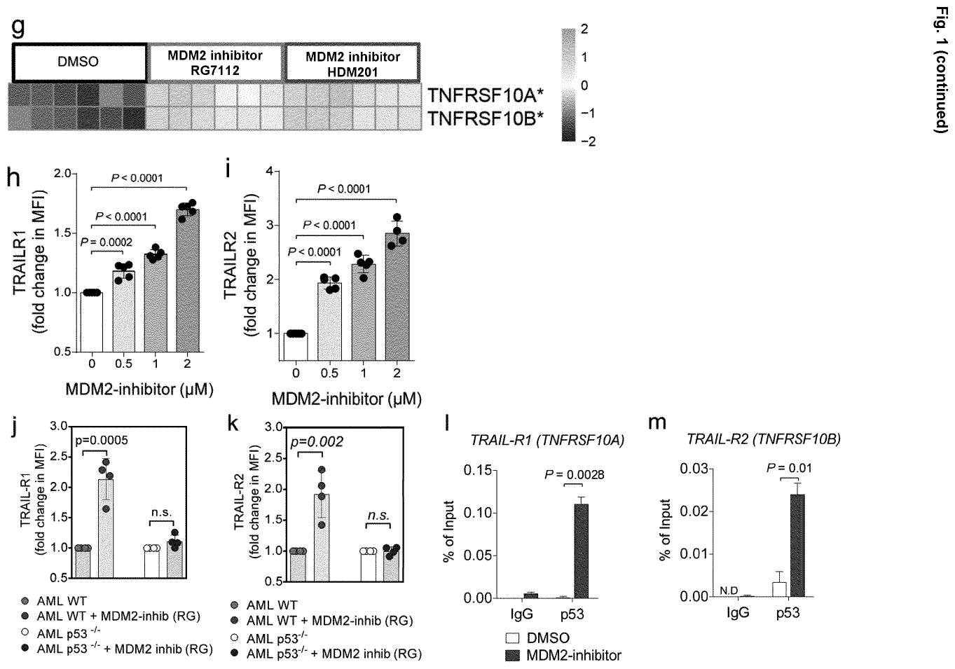

FIG. 1: MDM2-inhibition improves AML survival in multiple GVL mouse models

(a) Percentage survival of BALB/c recipient mice after transfer of AML WEHI-3B cells (BALB/c background) and allogeneic C57BL/6 BM is shown. As indicated, mice were injected with additional allogeneic T-cells (C57BL/6) and/or treated with either vehicle or MDM2-inhibitor RG-7112. n=9-10 independent animals per group are shown and p-values were calculated using the two-sided Mantel-Cox test.

(b) Percentage survival of C57BL/6 recipient mice after transfer of AML^{MLL-PTD FLT3-ITD} cells (C57BL/6 background) and allogeneic BALB/c BM is shown. As indicated, mice were injected with additional allogeneic T-cells (BALB/c) and/or treated with either vehicle or MDM2-inhibitor RG-7112. n=10 biologically independent animals from two experiments are shown and p-values were calculated using the two-sided Mantel-Cox test.

(c) Percentage survival of Rag2^{-/-}II2rγ^{-/-} recipient mice after transfer of human OCI-AML-3 cells is shown. As indicated, mice were injected with additional human T-cells (isolated from peripheral blood of healthy donors) and/or treated with either vehicle or MDM2-inhibitor RG-7112. n=12 biologically independent animals from three experiments are shown and p-values were calculated using the two-sided Mantel-Cox test.

(d) Percentage of specific lysis of isolated, CD3/28 and IL-2 expanded human T-cells in contact with OCI-AML3 cells is shown. OCI-AML3 cells were pre-treated with either DMSO or the MDM2-inhibitor RG-7112 and the E:T, effector (T-cell) to target (OCI-AML3 cell) ratio was varied between 10:1 and 1:1 as indicated. One representative experiment of three independent experiments is shown.

(e) Representative western blots showing the activation of Caspase-3 and loading control (β-Actin) in OCI-AML3 cells. OCI-AML3 cells exposed to DMSO or RG-7112 (1 μM) were co-cultured with activated T-cells at E:T ratio of 10:1 for 4 hours.

(f) The bar diagram indicates the ratio of the cleaved Caspase-3 to pro-Caspase-3 normalized to β-Actin. The values were normalized to the T cell only group (set as “1”).

(g) Microarray-based analysis of the expression level of TNFRSF10A and TNFRSF10B in OCI-AML3 cells after treatment with DMSO, RG-7112 (1 μM) or HDM-201 (200 nM) for 24 hours is shown as tile display from Robust Multichip Average (RMA) signal values, n=6 biologically independent samples per group.

(h) The graph shows the fold change of MFI for TRAIL-R1 expression on OCI-AML3 cells after treatment with the indicated concentrations of MDM2-inhibitor RG-7112 for 72 hours as mean±SEM from n=5 independent experiments. P-values were calculated using two-sided Student's unpaired t-test.

(i) The graph shows the fold change of MFI for TRAIL-R2 expression on OCI-AML3 cells after treatment with the indicated concentrations of MDM2-inhibitor RG-7112 for 72 hours as mean±SEM from n=5 independent experiments. P-values were calculated using two-sided Student's unpaired t-test.

(j, k) The graph shows fold change of MFI for TRAIL-R1 (j) or TRAIL-R2 (k) expression on OCI-AML3 (p53^{+/+}) or p53 knockout (p53^{-/-}) OCI-AML3 cells after treatment with the indicated concentrations of MDM2-inhibitor RG-7112 for 72 hours as mean±SEM from n=4 independent experiments. MFI of control-treated cells was set as 1.0. P-values were calculated using the two-sided Student's unpaired t-test.

(l, m) ChIP-qPCR analysis in OCI-AML3 cells treated with DMSO or 2 μM RG-7112 for 12 hours to detect the binding of p53 to the promoter of TRAIL-R1 (TNFRSF10A) (I) and TRAIL-R2 (TNFRSF10B) (m). Data are represented as percent input and are representative of three experiments; error bars, s.e.m. from three technical replicates. N.D, not detected.

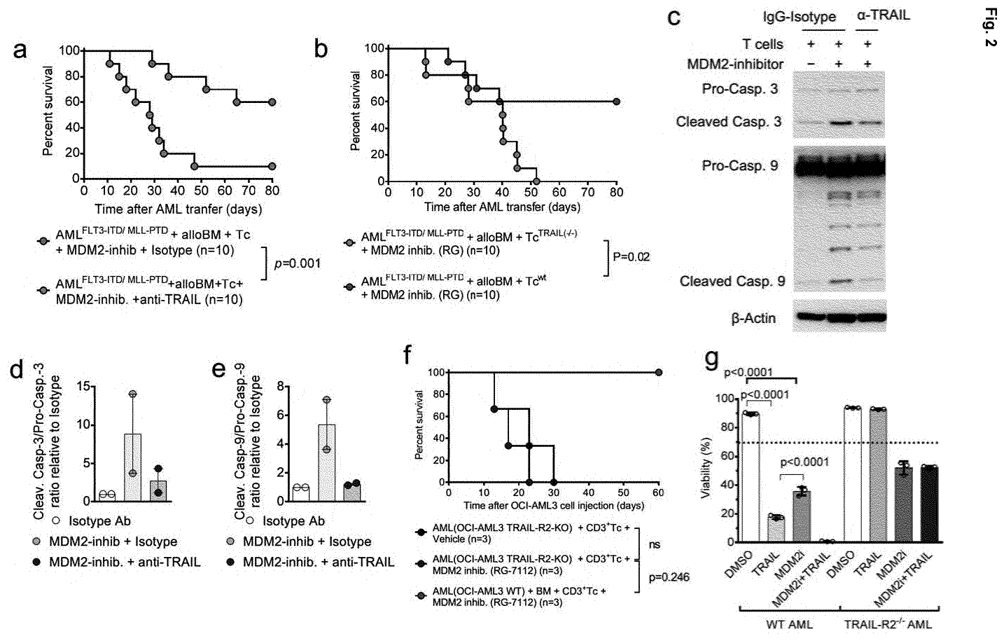

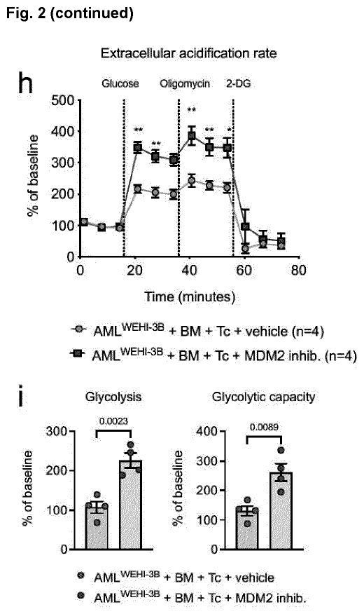

FIG. 2: MDM2-inhibition enhances TRAIL-R1/2 expression in a p53-dependent manner

(a) Percentage survival of C57BL/6 recipient mice after transfer of AML^{MLL-PTD FLT3-ITD} cells (C57BL/6 background) and allogeneic BALB/c BM is shown. Mice were injected with additional allogeneic T-cells (BALB/c), treated with the MDM2-inhibitor RG-7112 and with either anti-TRAIL-antibody or IgG-Isotype as indicated. n=10 independent animals from 2 experiments are shown and p-values were calculated using the two-sided Mantel-Cox test.

(b) Percentage survival of C57BL/6 recipient mice after transfer of AML^{MLL-PTD FLT3-ITD} cells (C57BL/6 background) and allogeneic BALB/c BM is shown. Mice were injected with additional allogeneic T-cells (BALB/c), either WT T-cells or TRAIL^{-/-} T-cells. n=10 independent animals from 2 experiments are shown and p-values were calculated using the two-sided Mantel-Cox test.

(c) Western blots showing the activation of Caspase-3, Caspase-9 and loading control (β-Actin) in OCI-AML3 cells. Activated T-cells were pretreated with 10 μg/ml anti-TRAIL, neutralizing antibody or IgG control for 1 hour and were co-cultured with OCI-AML3 cells exposed to DMSO or RG-7112 (1 μM) at E:T ratio of 10:1 for 4 hours.

(d) Quantification of the ratio of cleaved caspase-3/total caspase-3 normalized to isotype control. Each data point represents an independent biological replicate.

(e) Quantification of the ratio of cleaved caspase-9/total caspase-9 normalized to isotype control. Each data point represents an independent biological replicate.

(f) Survival of Rag2^{-/-}II2rγ^{-/-} mice receiving WT OCI-AML cells or TRAIL-R2 CRISPR-Cas knockout OCI-AML cells. Mice were additionally injected with primary human T-cells isolated from healthy donors and treated with vehicle or MDM2-inhibitor RG-7112. n=10 animals from two independent experiments are shown and p-values were calculated using the two-sided Mantel-Cox test.

(g) The bar diagram shows the viability of WT or TRAIL-R2 CRISPR-Cas knockout OCI-AML3 cells (TRAIL-R2^{-/-}) that were incubated with 1 μM of the MDM2-inhibitor RG7112, where indicated. After 48 hours limiting concentrations of hTRAIL (TNFSF 10) were added for 24 hours, where indicated. The viability of the AML cells was measured by flow cytometry. Mean of triplicates±SEM are displayed. P-values were calculated using two-sided Student's unpaired t-test.

(h) Extracellular acidification rate (ECAR) of CD8⁺ T-cells isolated from the spleen on day 12 following allo-HCT of WEHI-3B leukemia-bearing BALB/c mice that had undergone allo-HCT with C57BL/6 BM plus allogeneic C57BL/6 T-cells. Recipient mice were treated either with vehicle or MDM2-inhibitor RG-7112, as indicated. For each replicate, a normalization to the ECAR baseline value was performed. Mean value±SEM from n=4 biologically independent replicates, each replicate was generated by pooling the spleens from two mice. P-values were calculated using a two-sided unpaired Student's t-test.

(i) Glycolysis (calculated as the difference between ECAR after glucose injection, and basal ECAR) and glycolytic capacity (calculated as the difference between ECAR after oligomycin injection, and basal ECAR) of CD8⁺ T-cells isolated from BMT recipients as described in panel h. Mean value±SEM from n=4 biologically independent replicates, each replicate was generated by pooling the spleens from two mice. P-values were calculated using a two-sided unpaired Student's t-test.

(j) Fractional contribution of U-¹³C-glucose to glycolysis intermediates after ex vivo labeling of CD8⁺ T-cells isolated from BMT recipients as described in panel h. Each dot represents a single mouse. P-values were calculated using a two-sided unpaired Student's t-test, ns: not significant. Pathway schematic created with Biorender.com.

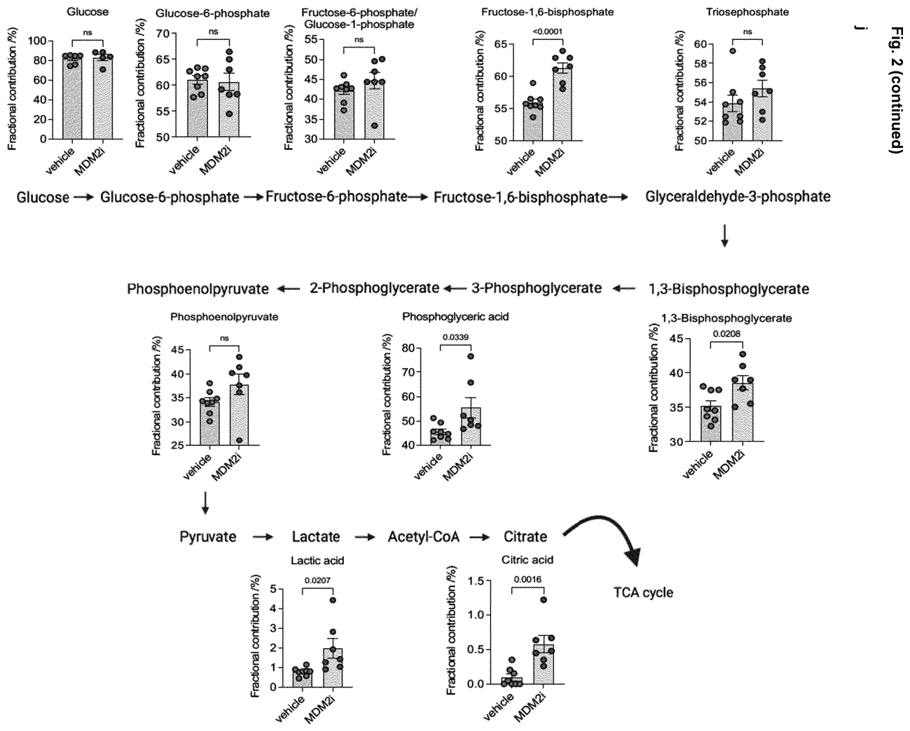

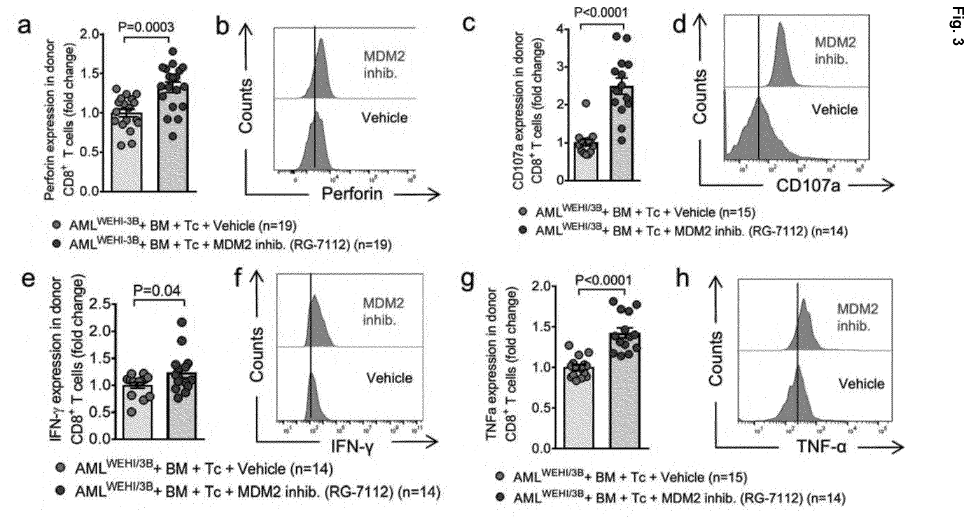

FIG. 3: MDM2-inhibition promotes cytotoxicity and longevity of donor T cells
(a-h) Scatter plots and representative histograms show expression of Perforin (a, b), CD107a (c, d), IFN-γ (e, f), TNF-α (g, h) of CD8⁺ T-cells isolated from spleen on day 12 following allo-HCT of WEHI-3B leukemia bearing BALB/c mice transplanted with C57BL/6 BM plus allogeneic C57BL/6 T-cells and treated with either vehicle or MDM2-inhibitor RG-7112. Mean value±SEM from n=14-19 biologically independent animals per group from 2 experiments are shown and p-values were calculated using two-sided Mann-Whitney U test.

(i) Percentage survival of C57BL/6 recipient mice after transfer of AML^{MLL-PTD FLT3-ITD} cells (C57BL/6 background) and BMT using allogeneic BALB/c BM is shown. Mice were injected with additional allogeneic T-cells (BALB/c) in day 2 after BMT. When indicated CD8 T-cells or NK cells were depleted. n=10 independent animals from 2 experiments are shown and p-values were calculated using the two-sided Mantel-Cox test.

(j) Percentage survival of C57BL/6 recipient mice after transfer of AML^{MLL-PTD FLT3-ITD} cells (C57BL/6 background) and allogeneic BALB/c BM is shown. Mice were injected with additional allogeneic T-cells (BALB/c), derived from previously challenged and treated (MDM2-inhibitor or vehicle) mice. n=10 independent animals from 2 experiments are shown and p-values were calculated using the two-sided Mantel-Cox test.

(k) UMAP showing the FIowSOM-guided manual metaclustering (A, top) and heatmap showing median marker expression (bottom) of splenic live CD45+ cells from allo-transplanted leukemia bearing BALB/c mice.

(l) UMAP showing the FIowSOM-guided manual metaclustering (A, top) and heatmap showing median marker expression (bottom) of donor-derived (H-2kb+) TCRb+CD8+ T cells from allo-transplanted leukemia bearing BALB/c mice treated with RG-7112 or vehicle as indicated.

(m) Quantification of donor-derived (H-2kb+) TCRb+CD8+CD27+ TIM3+ T cells from allo-transplanted leukemia bearing BALB/c mice treated with RG-7112 or vehicle as indicated.

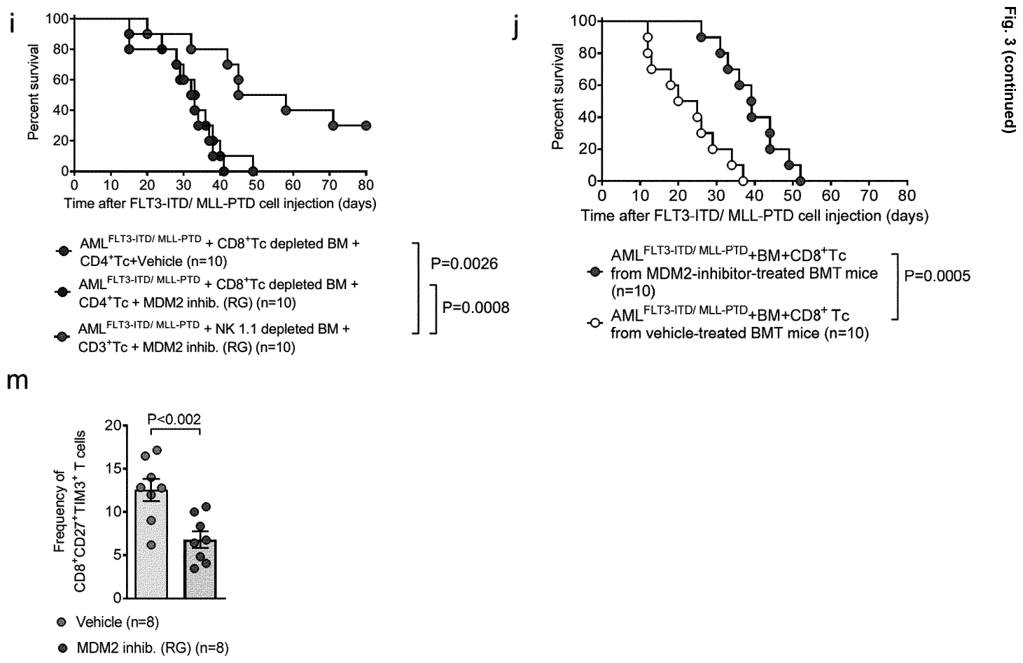

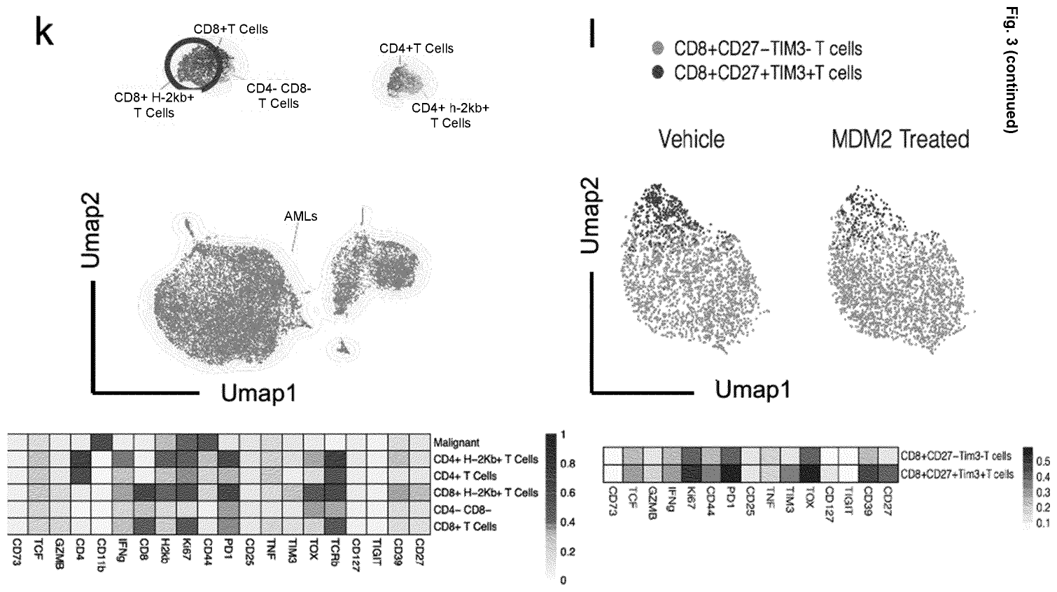

FIG. 4: MDM2-inhibition in primary human AML cells leads to TRAIL-1/2 expression

(a) The graph shows hTRAIL-R1 mRNA expression levels in primary human AML cells before or after in vitro treatment with RG-7112 (2 μM) for 12 hours normalized to hGapdh, as determined through qPCR. Each data point represents an individual sample of one independent patient. The experiments were performed independently and the results (mean±s.e.m.) were pooled.

(b) The graph shows a representative quantification of hTRAIL-R1 mRNA levels of primary AML blasts from patient-derived PBMCs after in vitro treatment with different concentrations of RG-7112 (0.5, 1 and 2 μM) for 12 hours.

(c) The graph shows hTRAIL-R2 mRNA expression levels in primary human AML cells before or after in vitro treatment with RG-7112 (2 μM) for 12 hours normalized to hGapdh, as determined through qPCR. Each data point represents an individual sample of one independent patient. The experiments were performed independently and the results (mean±s.e.m.) were pooled.

(d) The graph shows a representative quantification of hTRAIL-R2 mRNA levels of primary AML blasts from patient-derived PBMCs after in vitro treatment with different concentrations of RG-7112 (0.5, 1 and 2 μM) for 12 hours.

(e) Percentage survival of Rag2^{-/-}II2rγ^{-/-} recipient mice after transfer of primary human AML- cells is shown (patient #56). As indicated, mice were injected with additional human T-cells (isolated from the peripheral blood of an HLA non-matched healthy donor) and/or treated with either vehicle or MDM2-inhibitor RG-7112. n=10 independent animals are shown and p-values were calculated using the two-sided Mantel-Cox test.

(f) Percentage survival of Rag2^{-/-}II2rγ^{-/-} recipient mice after transfer of human WT or p53 knockdown (p53^{-/-}) OCI-AML-3 cells is shown. As indicated, mice were injected with additional human T-cells (isolated from the peripheral blood of an HLA non-matched healthy donor) and/or treated with either vehicle or MDM2-inhibitor RG-7112. n=10 biologically independent animals from two experiments are shown and p-values were calculated using the two-sided Mantel-Cox test.

(g) Representative western blots showing Caspase-8, Caspase-3, PARP and loading control (β-Actin) in human OCI-AML3 cells. OCI-AML3 cells exposed to DMSO or RG-7112 (1 μM) were co-cultured with activated T-cells at an E:T ratio of 10:1 for 4 hours. The values were normalized to β-Actin.

(h, i) Representative flow cytometry histogram (h) and fold change bar diagram (i) show the mean fluorescence intensity (MFI) for HLA-C expression on OCI-AML3 cells after treatment with the indicated concentrations of MDM2-inhibitor RG-7112 for 72 hours. Bar graphs show the mean±SEM from n=5-6 independent experiments. P-values were calculated using the two-sided Student's unpaired t-test.

(j, k) Representative flow cytometry histogram (j) and fold change bar diagram (k) show the mean fluorescence intensity (MFI) for HLA-DR expression on OCI-AML3 cells after treatment with the indicated concentrations of MDM2-inhibitor RG-7112 for 72 hours. Bar graphs show the mean±SEM from n=5-6 independent experiments. P-values were calculated using the two-sided Student's unpaired t-test.

(l, m) The graph shows fold change of MFI for HLA-C (I) HLA-DR (m) expression on OCI-AML3 (p53^{+/+}) or p53 knockdown (p53^{-/-}) OCI-AML3 cells after treatment with RG-7112 (2 μM) for 72 hours as mean±SEM from n=4 independent experiments. MFI of control-treated cells was set as 1.0. P-values were calculated using two-sided Student's unpaired t-test.

(n) Cumulative HLA-DR (MHC-II) levels of primary AML patient blasts after in vitro treatment with RG-7112 (2 μM) for 48 hours were determined by flow cytometry and are displayed as MFI of n=11 biologically independent patients. MFI of HLA-DR (MHC-II) from control treated cells was set as 1.0. P-values were calculated using the two-sided Wilcoxon matched-pairs signed rank test and is indicated in the graph.

(o) The representative histogram shows MFI for HLA-DR expression on primary AML blasts of a patient after in vitro treatment with the indicated concentrations of MDM2-inhibitor RG-7112 for 48 hours as mean±SEM from one experiment performed in triplicate. MFI from control treated cells was set as 1.0 and p-values were calculated using two-sided Student's unpaired t-test.

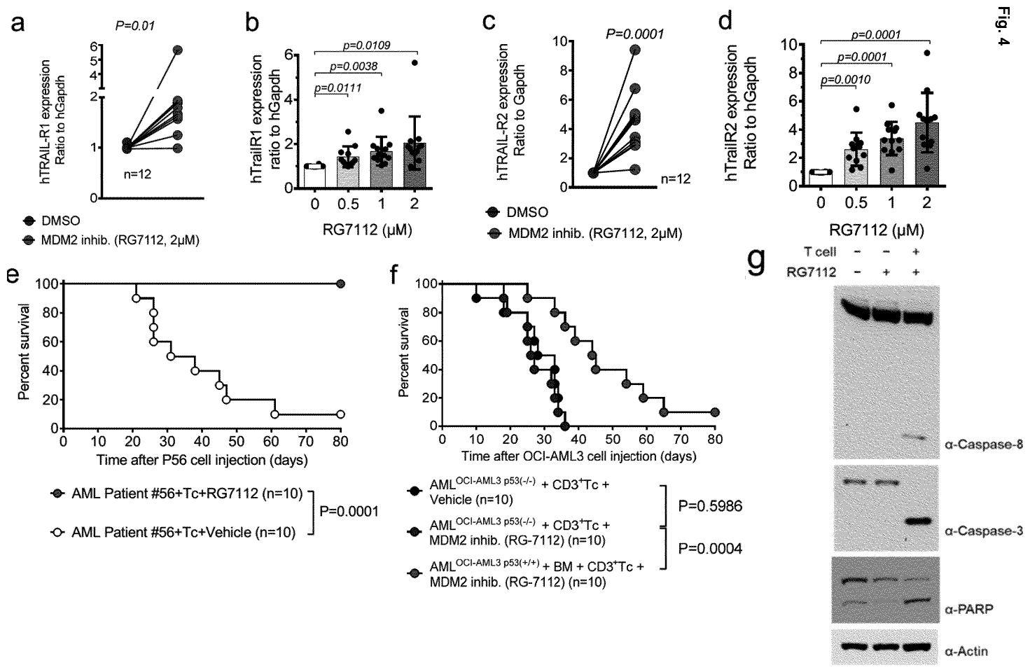

FIG. 5: GVHD histopathology scoring

(a-c) The scatter plot shows the histopathological scores from (a) liver, (b) colon, (c) small intestine isolated on day 12 after allo-HCT from C57BL/6 mice that had received BALB/c BM and T cells and were treated with either vehicle or the MDM2-inhibitor RG-7112. The P-values were calculated using the two-sided Mann-Whitney U test (non-significant (n.s.)).

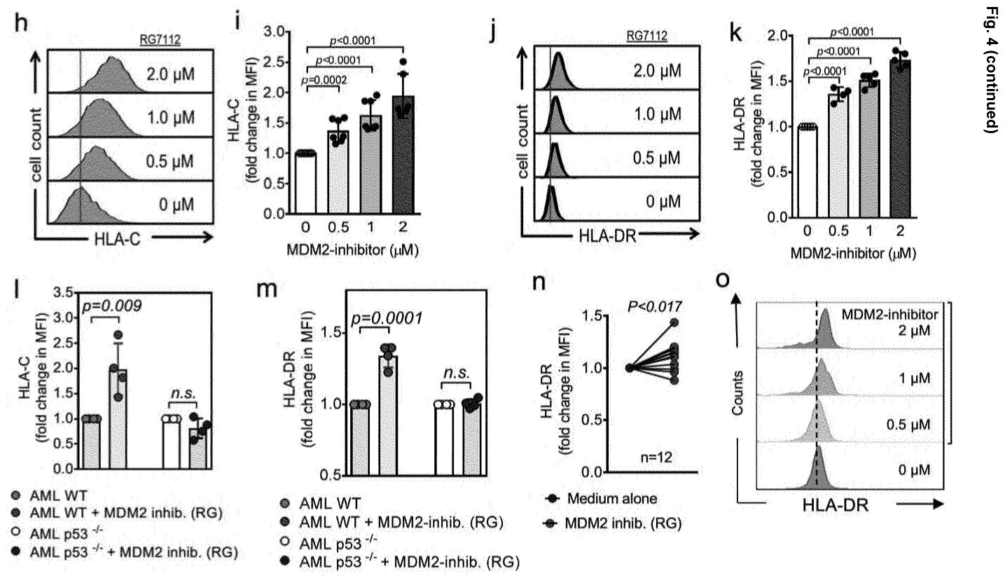

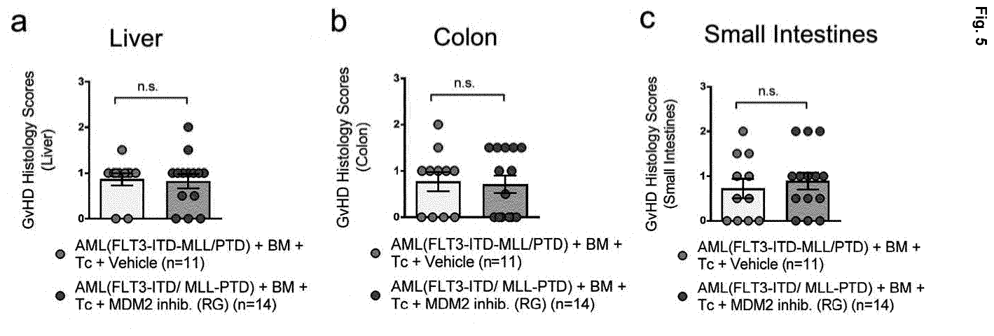

FIG. 6: TRAIL-R1/R2 mRNA and protein expression in human OCI-AML3 cells upon MDM2 inhibition with RG7112 or HDM201

(a) Representative flow cytometry histogram showing the mean fluorescence intensity (MFI) for TRAIL-R1 expression on OCI-AML3 cells after treatment with the indicated concentrations of MDM2-inhibitor RG-7112 for 72 hours. One of 5 independent biological replicates is shown.

(b) Representative flow cytometry histogram showing the mean fluorescence intensity (MFI) for TRAIL-R2 expression on OCI-AML3 cells after treatment with the indicated concentrations of MDM2-inhibitor RG-7112 for 72 hours. One of 5 independent biological replicates is shown.

(c-f) The graph shows fold-change of human TRAIL-R1 (hTRAILR1) RNA and hTRAILR2 RNA in OCI-AML3 cells after treatment with the indicated concentrations of MDM2-inhibitor RG-7112 for 6 h (c, d) or 12 h (e, f) as mean±SEM from n=3 independent experiments with each 2 technical replicates. RNA from control-treated cells was set as 1.0. P-values were calculated using two-sided Student's unpaired t-test.

(g, i) A representative flow cytometry histogram depicts the mean fluorescence intensity (MFI) for hTRAIL-R1 (g) and hTRAIL-R2 (i) expression on OCI-AML3 cells after treatment with the indicated concentrations of MDM2-inhibitor HDM-201 for 72 hours.

(h, j) The graph shows fold change of MFI of TRAIL-R1 (h) and TRAIL-R2 (j) expression on OCI-AML3 cells after treatment with the indicated concentrations of MDM2-inhibitor HDM201 for 72 hours as mean±SEM from n=5 independent experiments. MFI of control-treated cells was set as 1.0. P-values were calculated using two-sided Student's unpaired t-test.

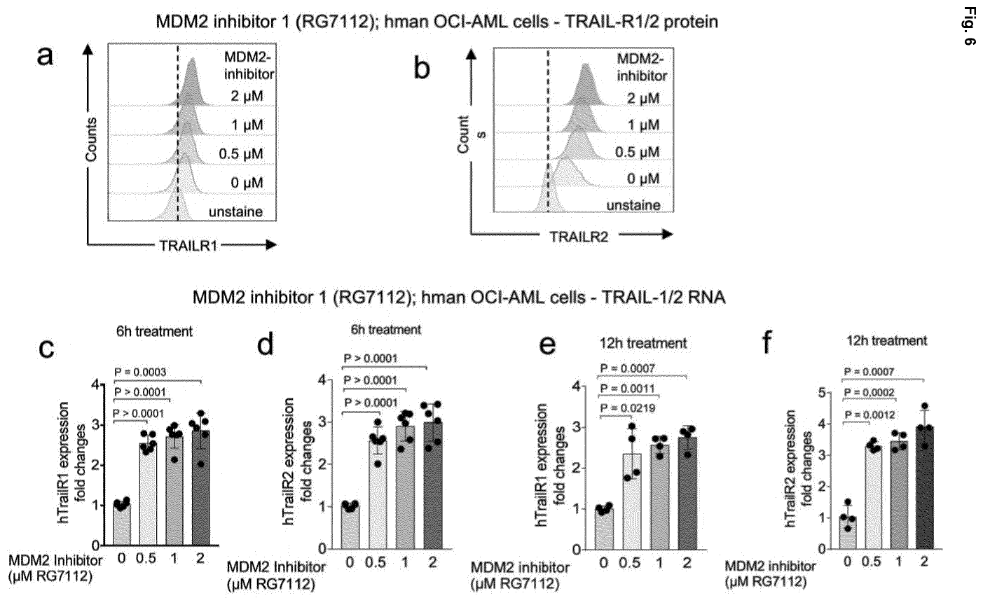

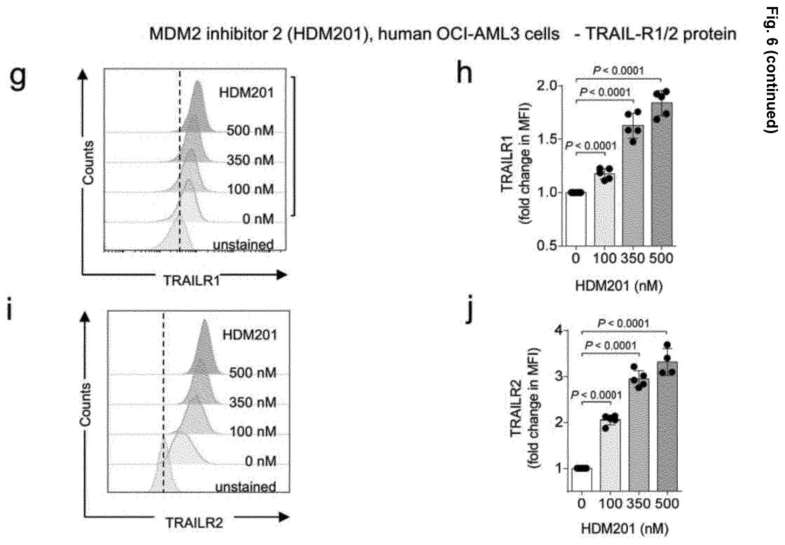

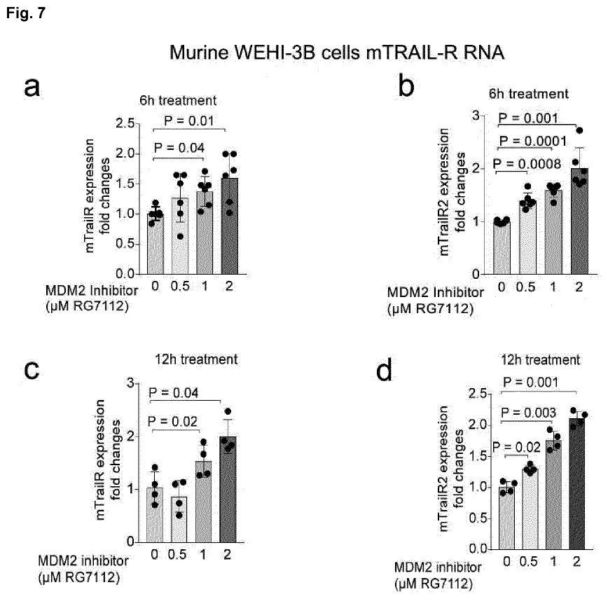

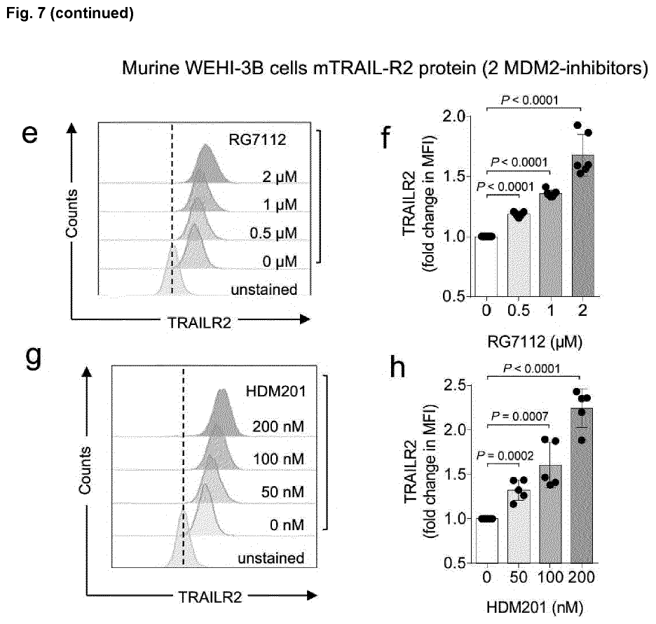

FIG. 7: TRAIL-R mRNA and protein expression in murine WEHI-3B cells

(a, b) The graph shows fold-change of mouse TRAIL-R (mTRAIL-R) RNA and mTRAIL-R2 RNA in WEHI-3B cells after treatment with the indicated concentrations of MDM2-inhibitor RG-7112 for 6 h as mean±SEM from n=4 independent experiments. RNA of DMSO-treated cells was set as 1.0. P-values were calculated using two-sided Student's unpaired t-test.

(c, d) The graph shows fold-change of mouse TRAIL-R (mTRAIL-R) RNA and mTRAIL-R2 RNA in WEHI-3B cells after treatment with the indicated concentrations of MDM2-inhibitor RG-7112 for 12 h as mean±SEM from n=4 independent experiments. RNA of DMSO-treated cells was set as 1.0. P-values were calculated using two-sided Student's unpaired t-test.

(e) A representative flow cytometry histogram depicts the mean fluorescence intensity (MFI) for TRAIL-R2 expression on WEHI-3B cells after treatment with the indicated concentrations of MDM2-inhibitor RG-7112 for 72 hours.

(f) The graph shows fold change of MFI for TRAIL-R2 expression on WEHI-3B cells after treatment with the indicated concentrations of MDM2-inhibitor RG-7112 for 72 hours as mean±SEM from n=5 independent experiments. MFI of control-treated cells was set at 1.0. P-values were calculated using the two-sided Student's unpaired t-test.

(g) A representative flow cytometry histogram depicts the mean fluorescence intensity (MFI) for TRAIL-R2 expression on WEHI-3B cells after treatment with the indicated concentrations of MDM2-inhibitor HDM201 for 72 hours.

(h) The graph shows fold change of MFI for TRAIL-R2 expression on WEHI-3B cells after treatment with the indicated concentrations of MDM2-inhibitor HDM201 for 72 hours as mean±SEM from n=5 independent experiments. MFI of control-treated cells was set at 1.0. P-values were calculated using the two-sided Student's unpaired t-test.

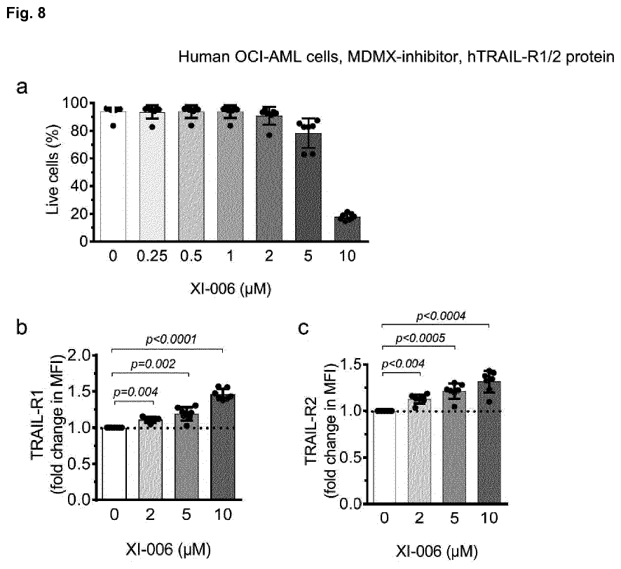

FIG. 8: XI-006 (MDMX-inhibitor) treatment leads to increased TRAIL-R1/R2 expression.

(a) The graph shows percentage of live (fixable viability dye negative) OCI-AML3 cells treated with the indicated concentrations of MDMX-inhibitor XI-006 for 72 hours as mean±SEM from n=7 independent experiments. P-values were calculated using the two-sided Student's unpaired t-test.

(b, c) The graph shows fold-change of MFI for TRAIL-R1 (b) and TRAIL-R2 (c) expression on OCI-AML3 cells after treatment with the indicated concentrations of MDMX-inhibitor XI-006 for 72 hours as mean±SEM from n=7 independent experiments. MFI of DMSO-treated cells was set as 1.0. P-values were calculated using the two-sided Student's unpaired t-test.

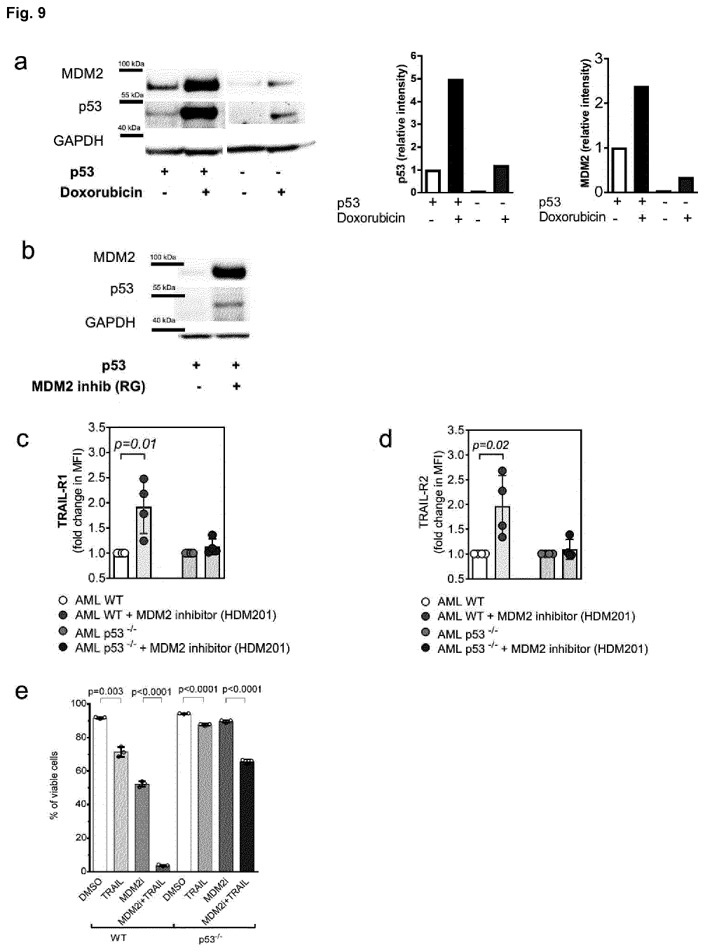

FIG. 9: HDM201 (MDM2-inhibitor) treatment increases TRAIL-R1/R2 expression on human OCI-AML3 cells in a p53-dependent manner

(a) Representative western blot (left panel) showing the expression of MDM2, p53 and loading control (GAPDH) in WT OCI-AML3 cells or p53 knockdown OCI-AML3 cells exposed to 1 mg/ml doxorubicin for 4 hours, when indicated. Right panel: Quantification of the relative intensity of the protein bands for each group.

(b) Representative western blot (left panel) showing the expression of MDM2, p53 and loading control (GAPDH) in OCI-AML3 cells exposed to 1 μM RG-7112 for 4 hours.

(c, d) The graph shows the fold change of MFI for TRAIL-R1 (c) and TRAIL-R2 (d) expression on wild type (WT) OCI-AML3 or p53 knockdown (p53^{-/-}) OCI-AML3 cells after treatment with the indicated concentrations of MDM2-inhibitor HDM201 for 72 hours as mean±SEM from n=4 independent experiments. MFI of control-treated cells was set as 1.0. P-values were calculated using the two-sided Student's unpaired t-test.

(e) The graph shows the percentage of viable cells. Where indicated wildtype OCI-AML3 (WT) or p53 knockout (p53^{-/-}) OCI-AML3 were incubated with 1 μM MDM2-inhibitor RG7112. After 48 hours limiting concentrations of hTRAIL (TNFSF 10) were added for 24 hours where indicated. Viability of cells was measured by flow cytometry. Mean of triplicates±SEM are displayed. P-values were calculated using two-sided Student's unpaired t-test.

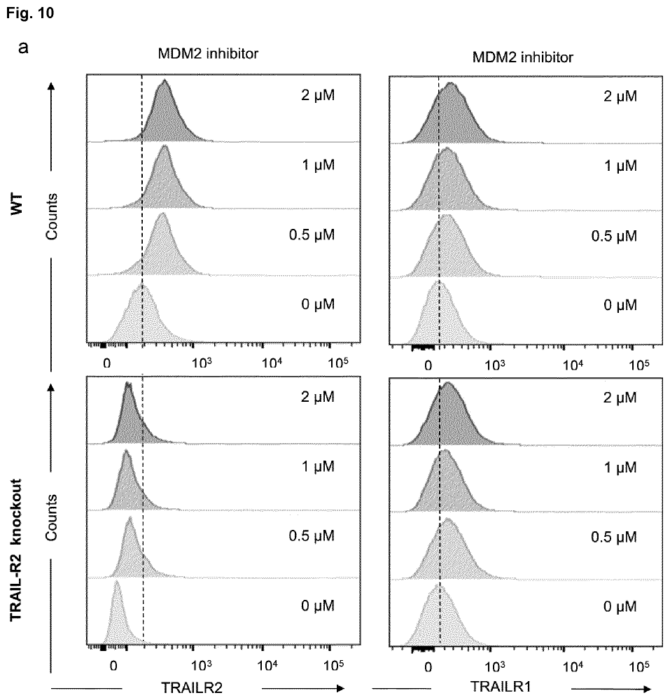

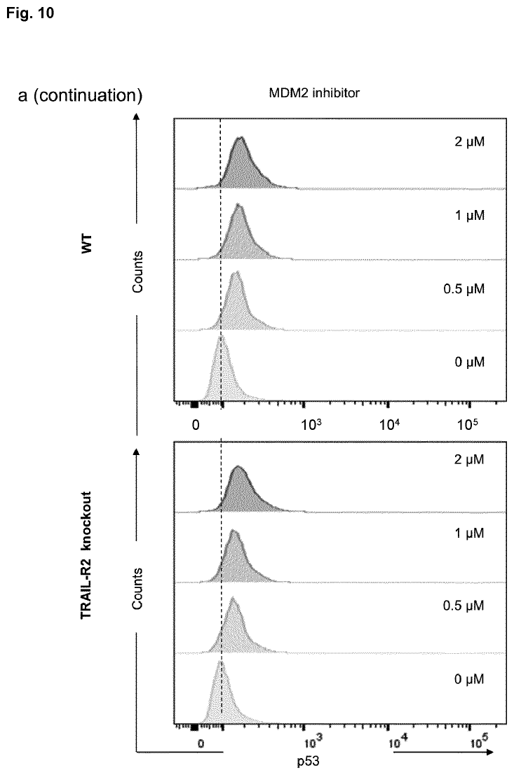

FIG. 10: TRAIL-R2 knockdown efficacy in OCI-AML3 cells and impact of MDM2 inhibition.

(a) A representative flow cytometry histogram depicts the mean fluorescence intensity (MFI) for hTRAIL-R2, hTRAIL-R1 and p53 expression on WT OCI-AML3 cells or upon hTRAIL-R2 knockout using CRISPR-Cas. Treatment with the indicated concentrations of MDM2-inhibitor RG7112 for 72 hours.

(b) The graph shows fold change of MFI of TRAIL-R2 expression on WT or TRAIL-R2 CRISPR-Cas knockout OCI-AML3 cells after treatment with the indicated concentrations of MDM2-inhibitor RG7112 for 72 hours as mean±SEM from n=2 independent experiments. P-values were calculated using two-sided Student's unpaired t-test.

(c) Viability of WT or TRAIL-R2 CRISPR-Cas knockout OCI-AML3 cells after treatment with optimal concentrations of hTRAIL (TNFSF 10) for 24 hours was measured by flow cytometry. Mean of triplicates±SEM are displayed. P-values were calculated using two-sided Student's unpaired t-test.

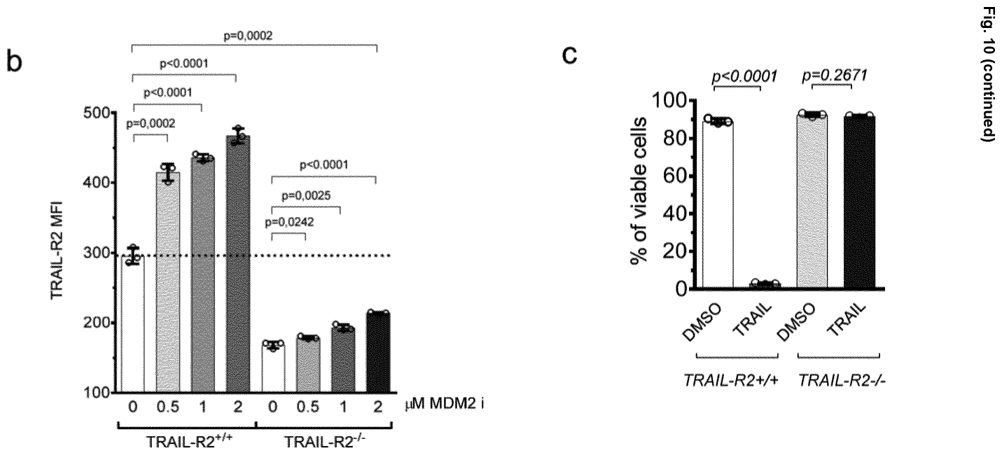

FIG. 11: MDM2 inhibition increases the metabolic activity of alloreactive T cells

(a-c) CD8⁺ T cells were enriched from the spleens of allo-HCT recipient mice, treated with MDM2 inhibitor. Polar metabolites were extracted and measured by LC-MS as described in the Supplementary Methods from n=8 mice treated with vehicle and n=7 mice treated with MDM2-inhibitor. (a) Volcano plot of 100 metabolites analyzed with a targeted approach. P-values were calculated using the unpaired two-tailed Student's t-test. (b) Heatmap of the 27 significantly regulated metabolites between “MDM2 inhibitor” and “vehicle” (p<0.05). Color scale indicates the normalized concentration in each sample. (c) Absolute abundance of metabolites from the pyrimidine biosynthesis pathway. Pathway scheme created with Biorender.com, *p<0.05, **p<0.01

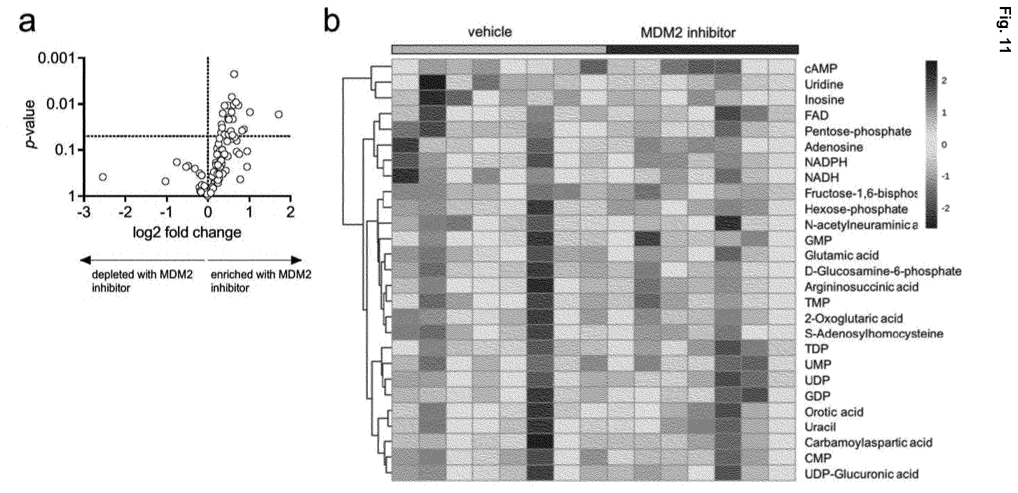

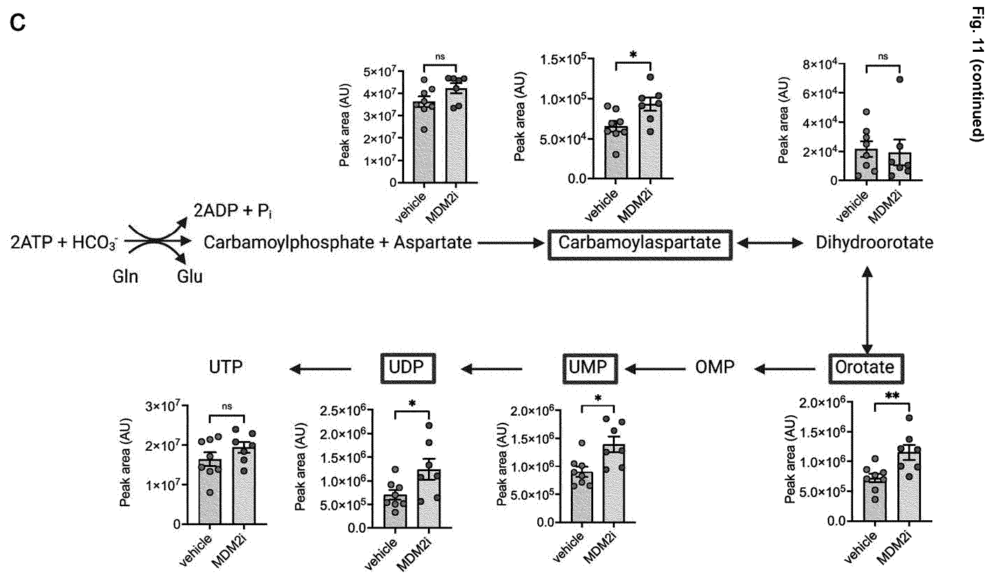

FIG. 12: Gating strategy for splenic H-2kb⁺CD8⁺ T cells and CD69 expression on CD8 T cells upon MDM2 inhibition in leukemia bearing mice.

(a) Flow cytometry plot showing the gating strategy to identify donor-derived (H-2kb⁺) CD3⁺CD8⁺ T cells from murine spleens. The gated cells were singlets, live (fixable viability dye negative), H-2kb⁺, CD45⁺, CD3⁺ and CD8⁺. The spleens were harvested from BALB/c mice which underwent TBI and were injected with C57BL/6 BM and WEHI-3B cells (d0). Mice were infused with allogeneic donor T cells (d2) and treated with 5 doses of RG-7112 every second day starting at d3.

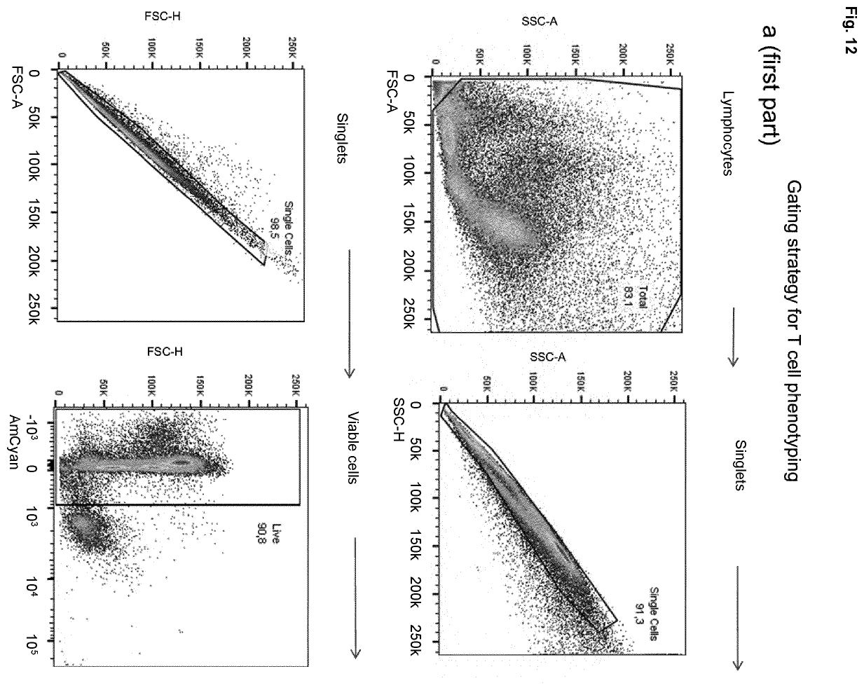

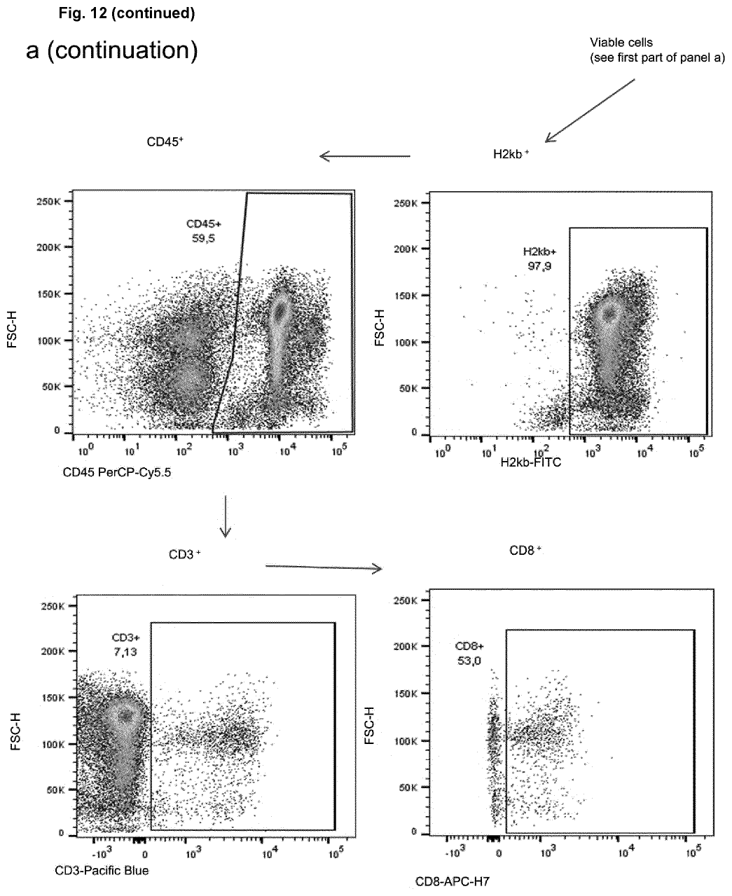

FIG. 13: Phenotype of T-cells isolated from MDM2-inhibitor treated mice that underwent allo-HCT.

(a) A representative flow cytometry histogram depicts the mean fluorescence intensity (MFI) and scatter plot showing fold-change of MFI for CD69 of all living donor (H-2kb⁺) CD8⁺ T cells from leukemia bearing BALB/c mice undergoing allo-HCT and being treated with vehicle. Mean value±SEM from n=14/15 biologically independent mice per group from 2 experiments are shown. MFI of vehicle-treated leukemia bearing mice was set as 1.0. P-values were calculated using the two-sided Mann-Whitney U test.

(b) Scatter plot showing the percentage of CD8⁺ cells of all living donor (H-2kb⁺) CD3⁺ T cells from allo-transplanted leukemia bearing BALB/c mice treated with RG-7112 or vehicle as indicated. Mean value±SEM from n=14/19 biologically independent mice per group from 3 experiments are shown. MFI of vehicle-treated leukemia bearing mice was set as 1.0. P-values were calculated using the two-sided Mann-Whitney U test. No difference in CD8 T-cells/all CD3 T-cells was detected.

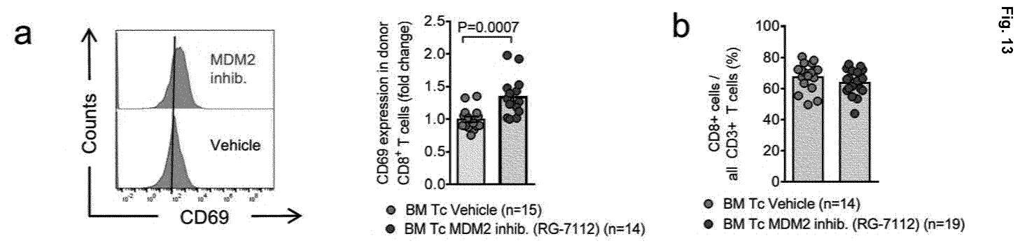

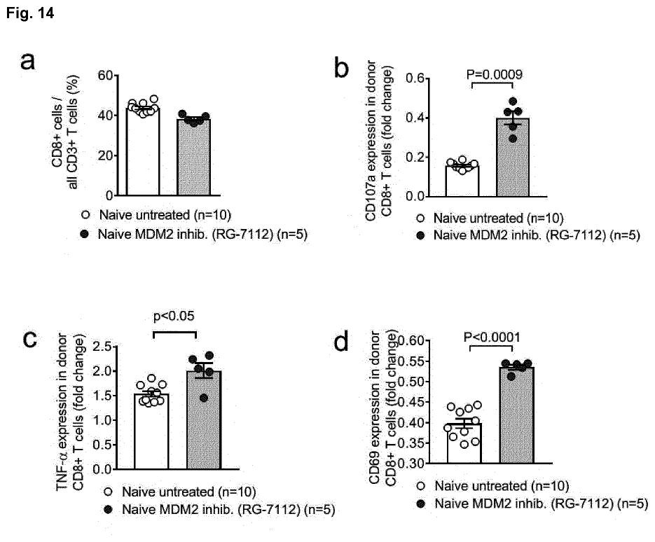

FIG. 14: MDM2 inhibition promotes T cell cytotoxicity in naive mice

(a-d) Flow cytometry analysis of splenocytes from naïve C57BL/6 mice treated with 5 doses of RG-7112 or vehicle every second day. The time point of analysis was 1 day after the last treatment.

(a) Scatter plot showing the percentage of CD8⁺ cells of all living donor (H-2kb⁺) CD3⁺ T cells from untreated naïve C57BL/6 mice treated with RG-7112 or vehicle as indicated. Mean value±SEM from n=5/10 biologically independent mice per group from 2 experiments are shown. MFI of vehicle-treated leukemia bearing mice was set as 1.0. P-values were calculated using two-sided Mann-Whitney U test.

(b-d) Scatter plots showing fold-change of MFI for CD107a (b), TNFα (c) and CD69 (d) of all living donor (H-2kb⁺) CD8⁺CD3⁺ T cells from untreated naïve C57BL/6 mice treated with vehicle. Mean value±SEM from n=5/10 biologically independent mice per group from 2 experiments are shown. MFI of vehicle-treated leukemia bearing mice was set as 1.0. P-values were calculated using the two-sided Mann-Whitney U test.

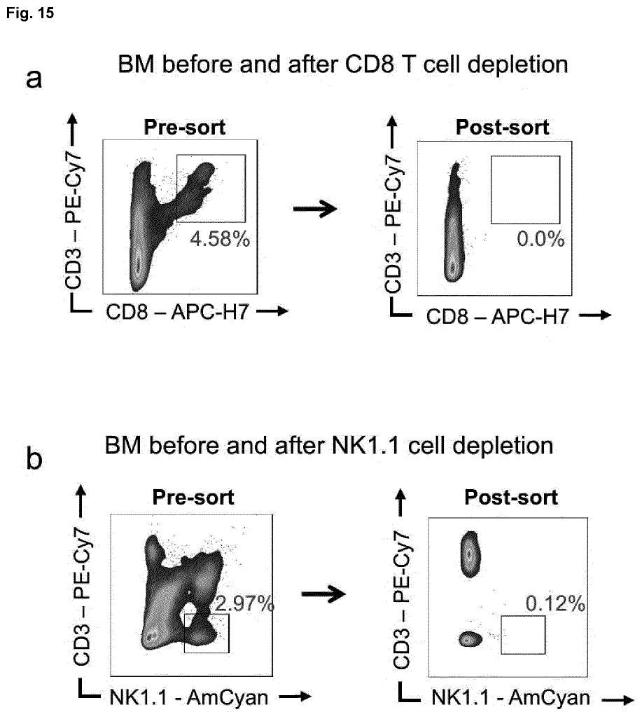

FIG. 15: Purity of BM graft before and after depletion of CD8⁺ T cells or NK1.1⁺ cells.

(a) A representative flow cytometry plot indicating the BM purity before and after depletion of CD8+ T cells via fluorescence-activated cell sorting. The indicated sorted cells were used for BM CD8⁺-depleted survival experiments. Similar results were obtained in two independent experiments.

(b) A representative flow cytometry plot indicating the BM purity before and after depletion of NK1.1⁺ cells via fluorescence-activated cell sorting. The indicated sorted cells were used for BM NK-cell-depleted survival experiments. Similar results were obtained in two independent experiments.

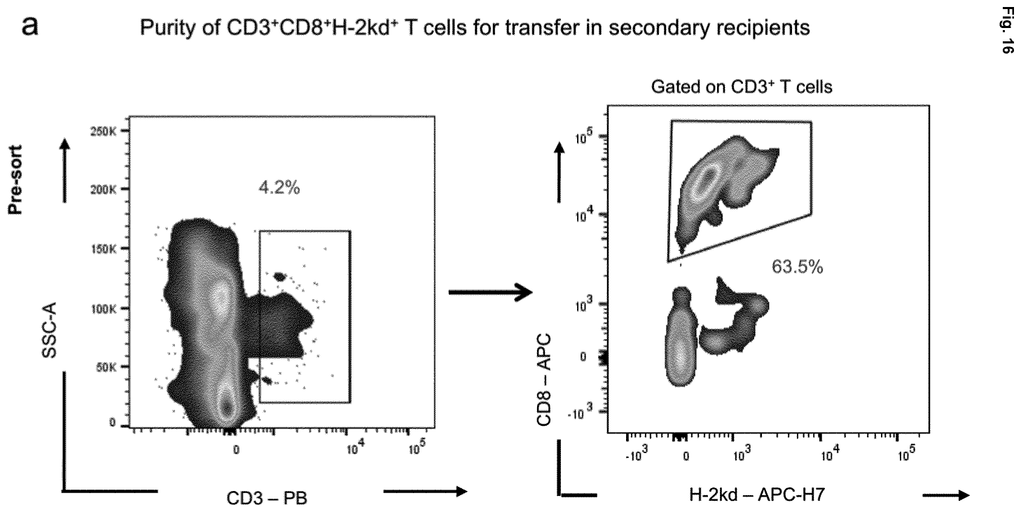

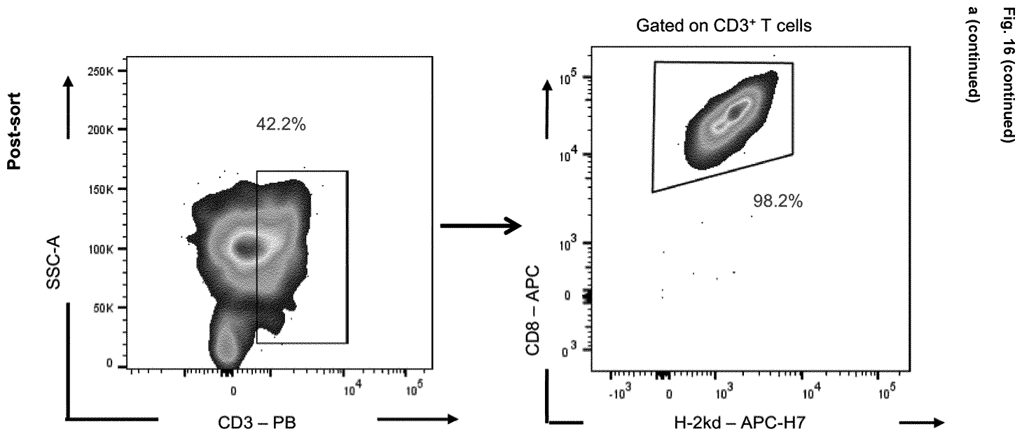

FIG. 16: Purity of CD3⁺CD8⁺H-2kd⁺ T cells for transfer in secondary recipients

(a) A representative flow cytometry plot indicating the purity of splenic CD3⁺H-2kd⁺CD8⁺ T cells (of all living cells) which were reisolated from C57BL/6 mice transplanted with BALB/c BM, murine AML^{MLL-PTD/FLT3-ITD} cells (d0) and allogeneic BALB/c T cells (d2). Mice received 5 doses of RG-7112 or vehicle every second day from d3 onwards. Splenocytes were harvested on d12 following allo-HCT. Sorted cells were used for recall immunity survival experiments. Similar results were obtained in three independent experiments.

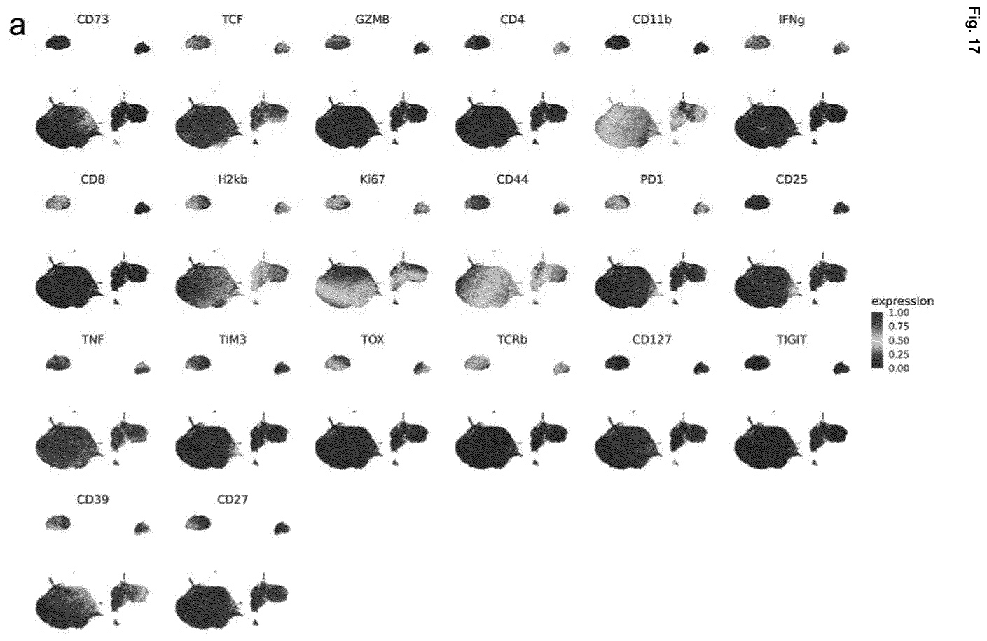

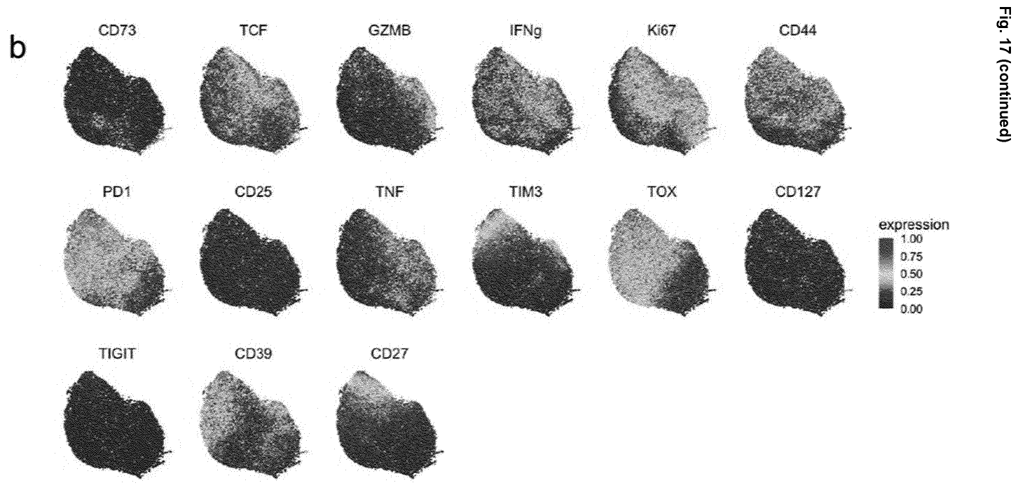

FIG. 17: Umap showing the marker expression on CD45⁺ and donor-derived (H-2kb⁺) TCRβ⁺CD8⁺ T cells.

(a, b) Umap diagram showing the marker expression on randomly selected live CD45⁺ cells (a) and donor-derived (H-2kb⁺) TCRβ⁺CD8⁺ T cells (b) from leukemia bearing BALB/c mice that had undergone allo-HCT.

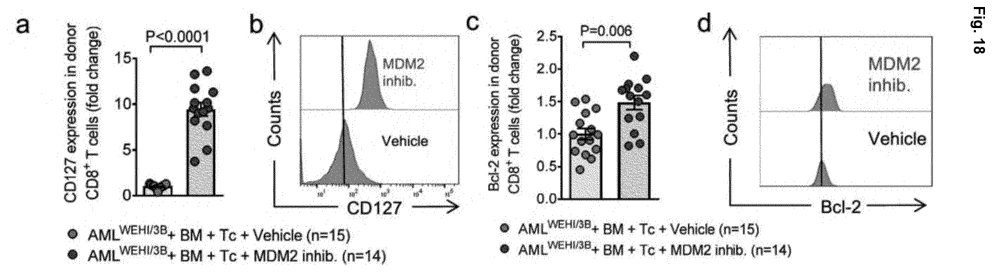

FIG. 18: MDM2 inhibition leads to increased levels of CD127 and Bcl-2 in CD8 T cells.

(a-d) Scatter plots and representative histograms show expression of CD127 (k, l), Bcl-2 (m, n) of CD8⁺ T-cells isolated from spleen on day 12 following allo-HCT of WEHI-3B leukemia bearing BALB/c mice transplanted with C57BL/6 BM plus allogeneic C57BL/6 T-cells and treated with either vehicle or MDM2-inhibitor RG-7112. Mean value±SEM from n=14-19 biologically independent animals per group from 2 experiments are shown and p-value was calculated using two-sided Mann-Whitney U test.

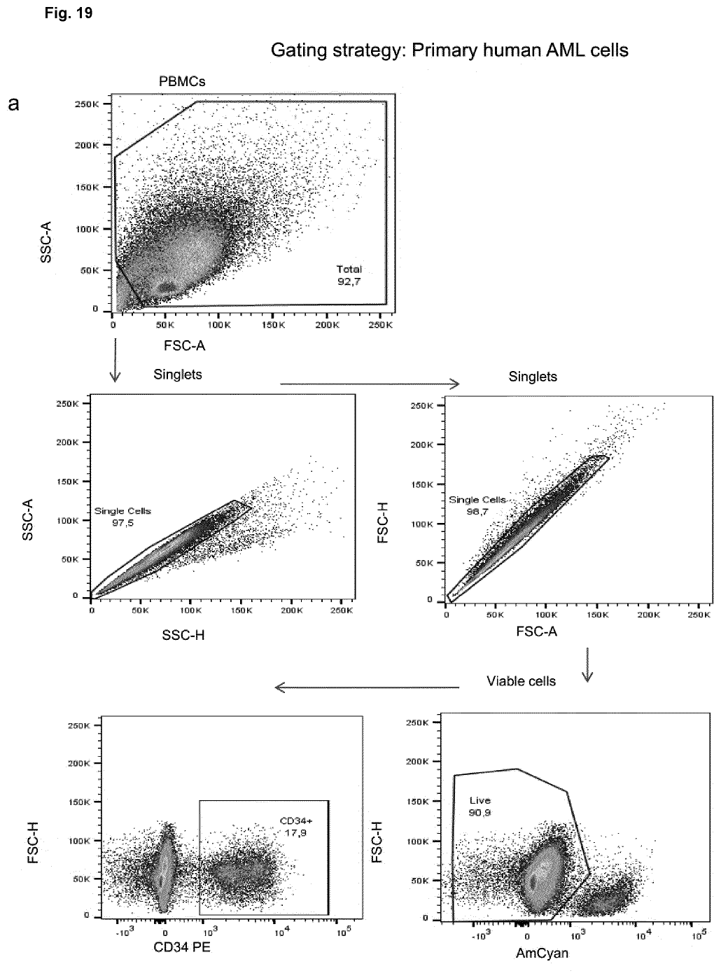

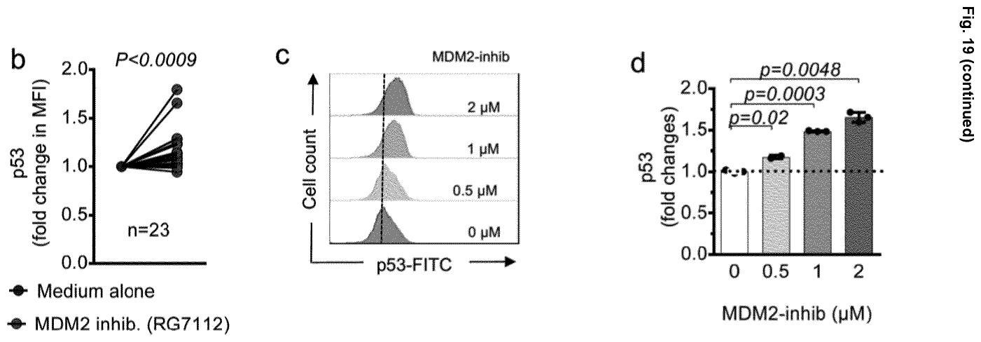

FIG. 19: Gating strategy to identify primary AML blasts in PBMCs and MDM2 inhibition increases p53 in primary AML patient blasts.

(a) Flow cytometry plot showing the gating strategy to identify primary AML blasts in patient-derived PBMCs. The gated cells were singlets, live (fixable viability dye negative) and either positive for the marker CD34⁺ or CD117 (cKIT)⁺ (here gating for CD34-positive cells is shown). The marker was chosen based on the informative marker expression on the AML cells at primary diagnosis.

(b) Cumulative p53 levels of primary AML patient blasts after in vitro treatment with RG-7112 (2 μM) for 48 hours were determined by flow cytometry and are displayed as MFI of n=23 biologically independent patients. MFI of p53 from control treated cells was set as 1.0. P-value was calculated using the two-sided Wilcoxon matched-pairs signed rank test and is indicated in the graph.

(c, d) The histogram (c) and graph (d) show fold-change of MFI for p53 expression on primary AML blasts of a representative patient after treatment with the indicated concentrations of MDM2-inhibitor RG-7112 for 48 hours as mean±SEM from one experiment performed in triplicate. MFI from control treated cells was set as 1.0 and p-values were calculated using the two-sided Student's unpaired t-test.

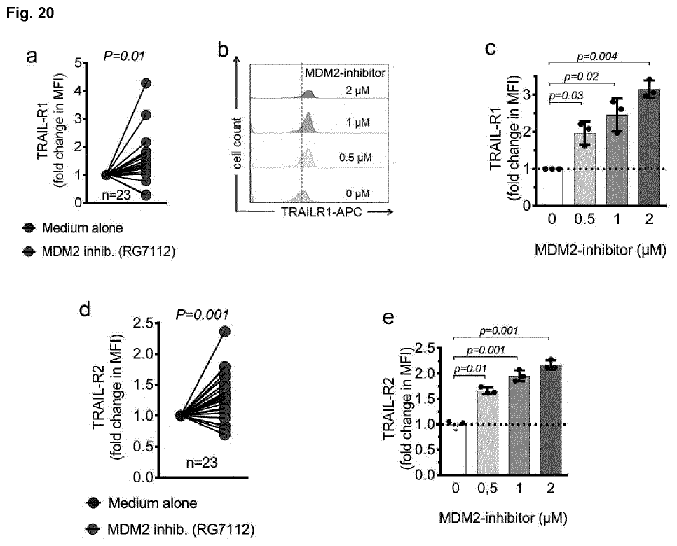

FIG. 20: MDM2 inhibition leads to TRAIL-R1/R2 protein upregulation in primary AML patient blasts.

(a) Cumulative TRAIL-R1 levels of primary AML patient blasts after in vitro treatment with RG-7112 (2 μM) for 48 hours were determined by flow cytometry and are displayed as MFI of n=23 independent patients. MFI of TRAIL-R1 from control treated cells was set as 1.0. P-values were calculated using the two-sided Wilcoxon matched-pairs signed rank test and is indicated in the graph.

(b, c) The histogram (b) and graph (c) show fold change of MFI for TRAIL-R1 expression on primary AML blasts of a representative patient after treatment with the indicated concentrations of MDM2-inhibitor RG-7112 for 48 hours as mean±SEM from one experiment performed in triplicate. MFI from control treated cells was set as 1.0 and p-values were calculated using the two-sided Student's unpaired t-test.

(d) Cumulative TRAIL-R2 levels of primary AML patient blasts after in vitro treatment with RG-7112 (2 μM) for 48 hours were determined by flow cytometry and are displayed as MFI of n=22 biologically independent patients. MFI of TRAIL-R1 from control treated cells was set as 1.0. P-values were calculated using the two-sided Wilcoxon matched-pairs signed rank test and is indicated in the graph.

(e) The histogram shows fold change of MFI for TRAIL-R2 expression on primary AML blasts of a representative patient after treatment with the indicated concentrations of MDM2-inhibitor RG-7112 for 48 hours as mean±SEM from one experiment performed in triplicate. MFI from control treated cells was set as 1.0 and p-values were calculated using the two-sided Student's unpaired t-test.

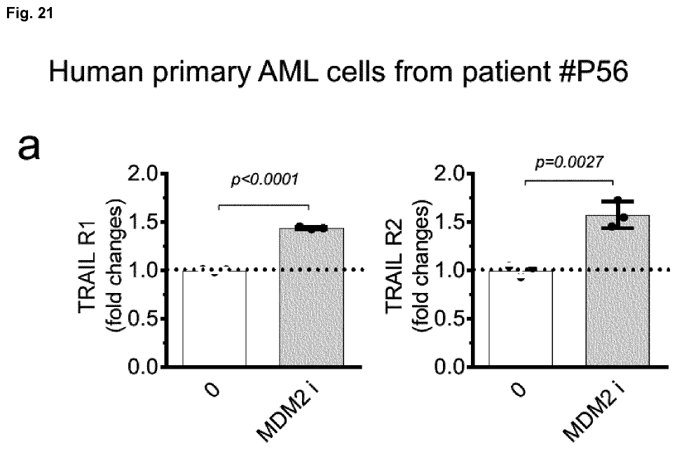

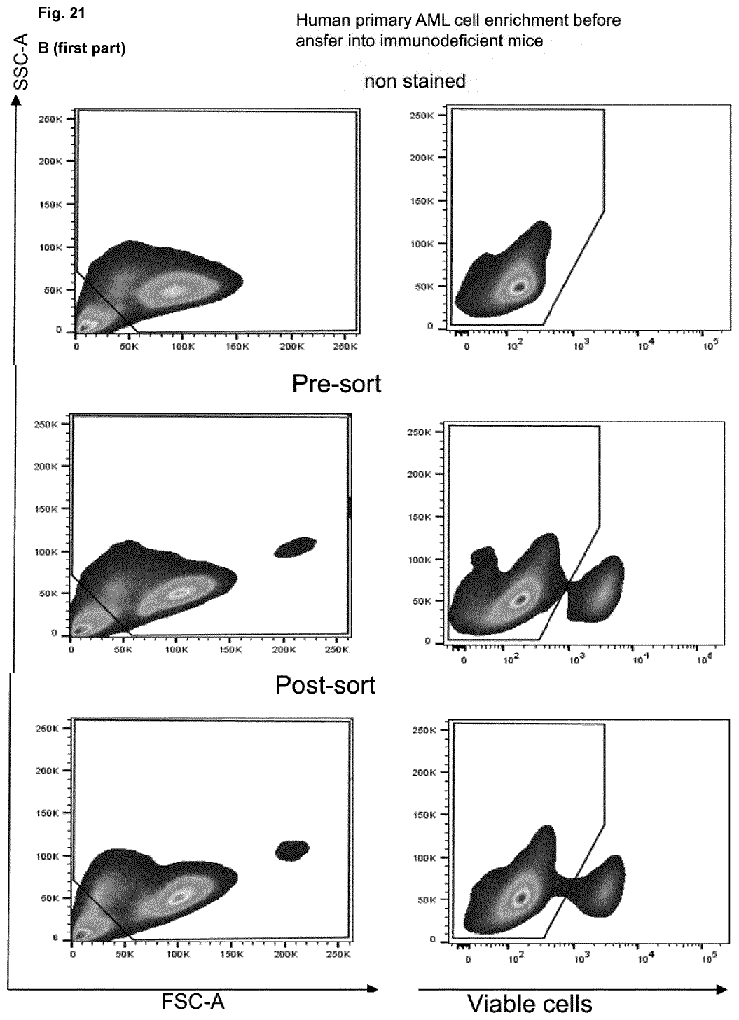

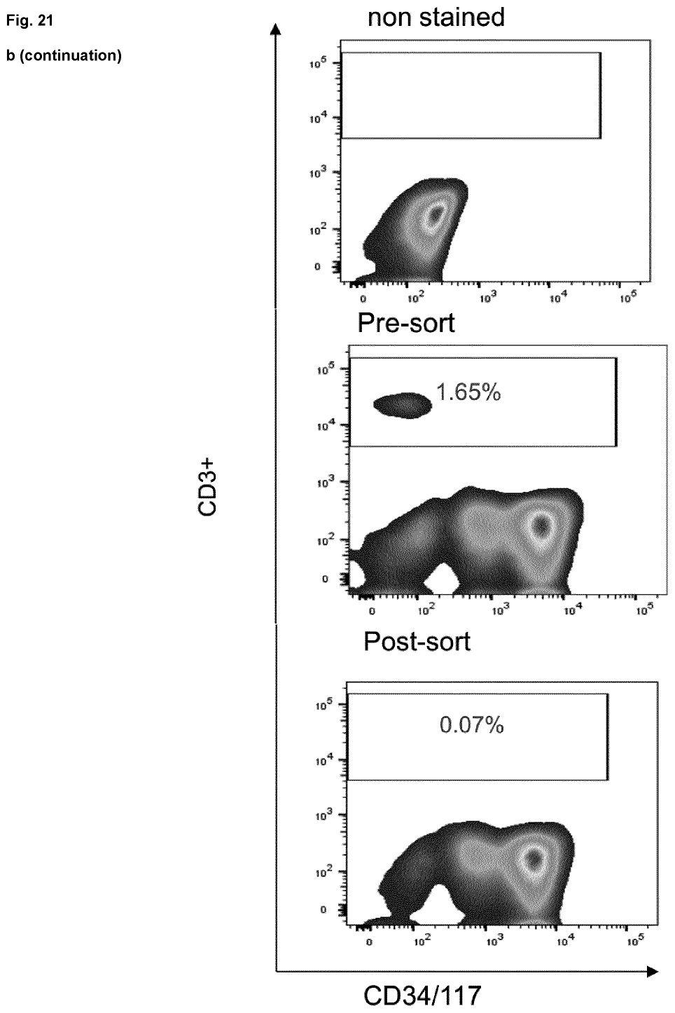

FIG. 21: MDM2 inhibition leads to TRAIL-R1/R2 mRNA upregulation in primary AML blasts of patient #56. Purity control of AML xenograft mouse models using primary AML blasts of patient #56

(a) The bar diagram shows TRAIL-R1/R2 protein levels (MFI) upon exposure of primary AML blasts of patient #56 to MDM2-inhibition (RG). The human leukemia cells (without prior MDM2 inhibition) were used for the survival studies in the xenograft experiment (shown in FIG. 4).

(b) Representative flow cytometry plots indicating AML cell enrichment before transfer into immunodeficient mice. The gated cells were singlets, live (fixable viability dye negative) and human CD45⁺.

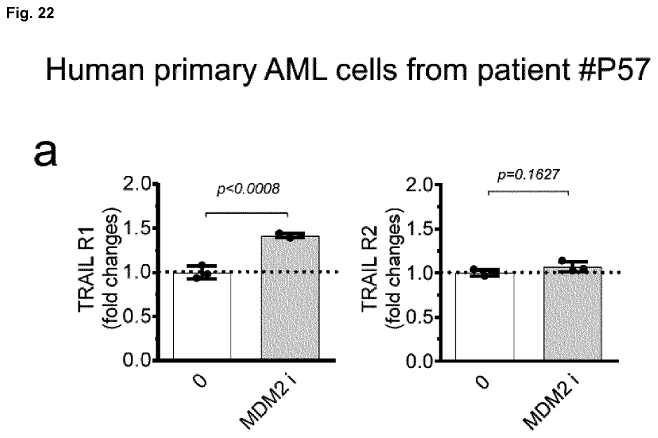

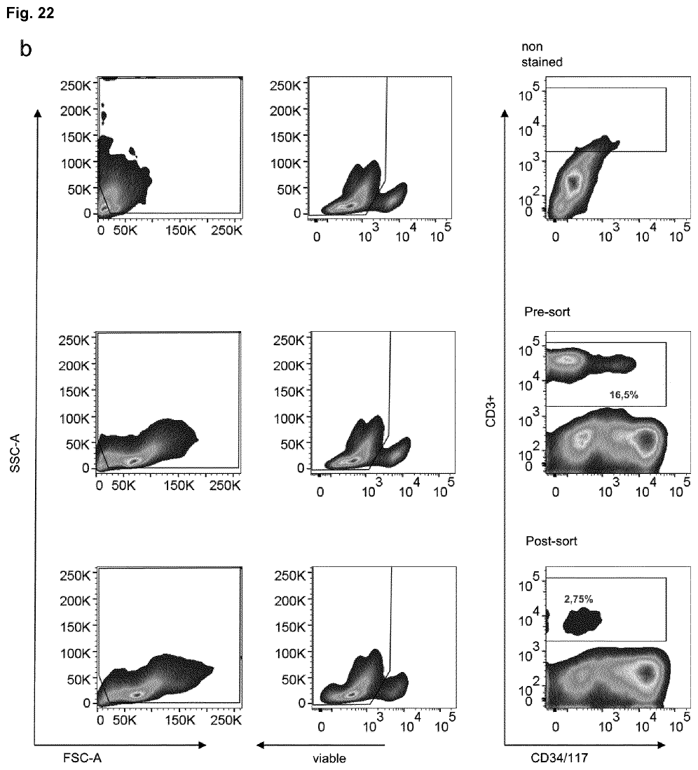

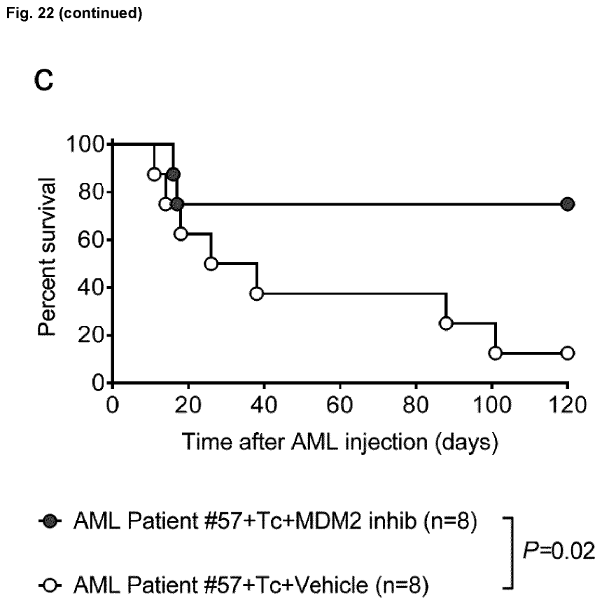

FIG. 22: MDM2 inhibition leads to TRAIL-R1/R2 mRNA upregulation in primary AML blasts of patient #57. Purity of the AML cells before transfer and survival studies.

(a) The bar diagram shows TRAIL-R1/R2 protein levels (MFI) upon exposure of primary AML blasts of patient #57 to MDM2-inhibition (RG). The human leukemia cells (without prior MDM2 inhibition) were used for the survival studies in the xenograft experiment.

(b) A representative flow cytometry plots indicating the AML cell enrichment before transfer into immunodeficient Rag2^{-/-}II2rγ^{-/-} mice. The gated cells were singlets, live (fixable viability dye negative) and human CD45⁺.

(c) Percentage survival of Rag2^{-/-}II2rγ^{-/-} recipient mice after transfer of primary human AML-cells is shown (patient #57). As indicated, mice were injected with additional human T-cells (isolated from peripheral blood of healthy donors) and/or treated with either vehicle or MDM2-inhibitor RG-7112. n=8 independent animals from three experiments are shown and p-values were calculated using the two-sided Mantel-Cox test.

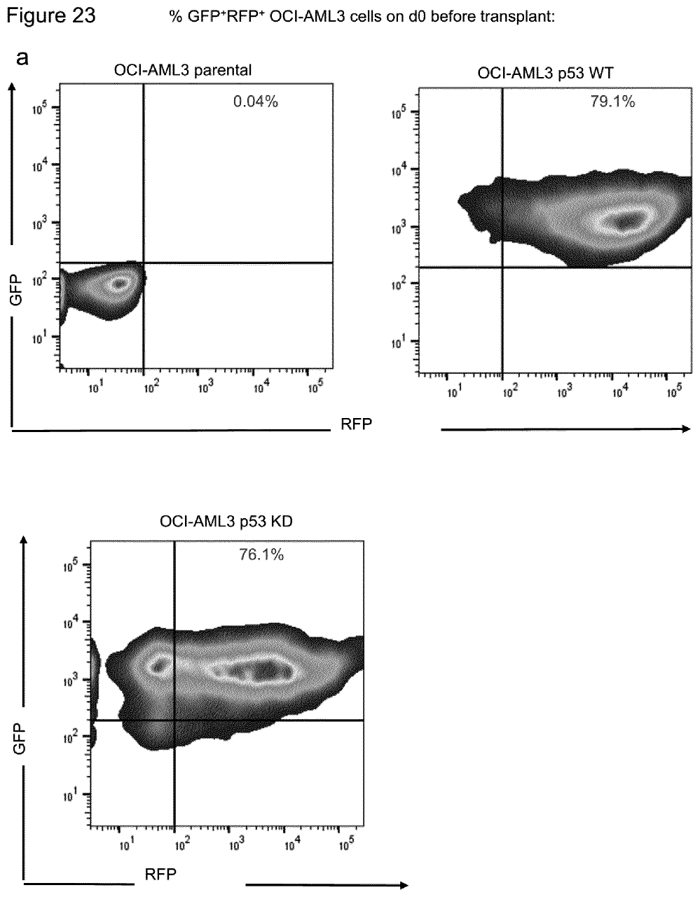

FIG. 23: P53 knockdown efficacy in p53^{-/-} OCl-AML3 cells pre-transplant.

(a) A representative flow cytometry plot indicating the p53-knockdown efficacy in OCI-AML3 cells pre-transplant. Cells were cultured in 20% FCS RPMI media containing 1 μg/ml doxycycline and 50 μg/ml blasticidin for a minimum of 7 days. The gated cells were singlets and live (fixable viability dye negative). Cells with stable knockdown efficiencies are shown as GFP⁺RFP⁺ population. Similar results were obtained in two independent experiments.

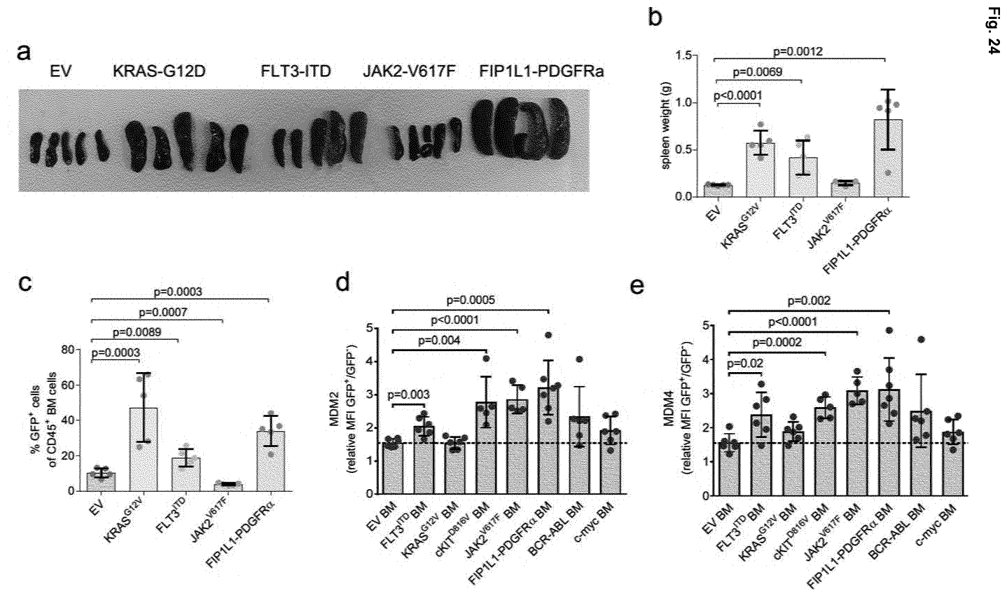

FIG. 24: The oncogenic mutations FIP1L1-PDGFR-a and cKIT-D816V that increase MDM2 in myeloid BM cells renders the AML sensitive to MDM2-inhibitor/T-cell effects.

(a) Spleens of mice 26 days after transfer of 33 000 primary murine BM cells transduced with FLT3-ITD, KRAS-G12D, cKIT-D816V, JAK2-V617F or FIP1L1-PDGFR-α and 5*10⁶ BALB/c BM cells.

(b) The bar diagram shows the weights of the spleens of the different groups shown in (a)

(c) Percentage of oncogene transduced (GFP⁺) cells of all CD45⁺ cells in the BM of mice from (a), quantified by flow cytometry.

(d) MDM2 protein (MFI) in primary murine BM cells transduced with FLT3-ITD, KRAS-G12D, cKIT-D816V, JAK2-V617F, FIP1 L1-PDGFR-α, BCR-ABL or c-myc as indicated.

(e) MDM4 protein (MFI) in primary BM cells transduced with FLT3-ITD, KRAS-G12D, cKIT-D816V, JAK2-V617F, FIP1L1-PDGFR-α, BCR-ABL or c-myc as indicated.

(f) Western blot showing the amount of MDM2 and loading control (β-Actin) in primary murine BM cells transduced with FLT3-ITD, KRAS-G12D, cKIT-D816V, JAK2-V617F, FIP1L1-PDGFR-α, BCR-ABL or c-myc as indicated.

(g) The bar diagram shows the ratio of MDM2/β-Actin in primary murine BM cells transduced with FLT3-ITD, KRAS-G12D, cKIT-D816V, JAK2-V617F, FIP1L1-PDGFR-α, BCR-ABL or c-myc. The ratio is normalized to EV (empty vector). The experiment was performed two times using biological repeats (BM from different mice) and the data were pooled.

(h) Percentage survival of BALB/c recipient mice after transfer of FIP1L1-PDGFR-α-tg transduced BM cells (BALB/c background) and 30 days afterwards allogeneic C57BL/6 BM is shown. Mice received allogeneic C57BL/6 CD3⁺ T cells at day two post BM transfer and were treated either with vehicle or MDM2-inhibitor.

(i) Percentage survival of BALB/c recipient mice after transfer of cKIT-D816V-tg transduced BM cells (BALB/c background) and 30 days afterwards allogeneic C57BL/6 BM is shown. Mice received allogeneic C57BL/6 CD3⁺ T cells at day two post BM transfer and were treated either with vehicle or MDM2-inhibitor.

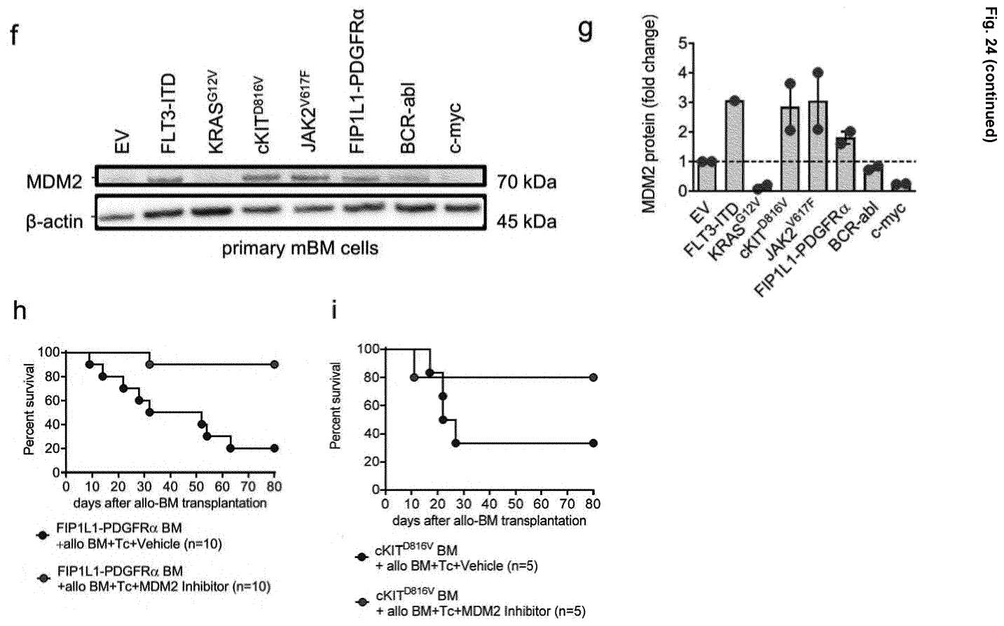

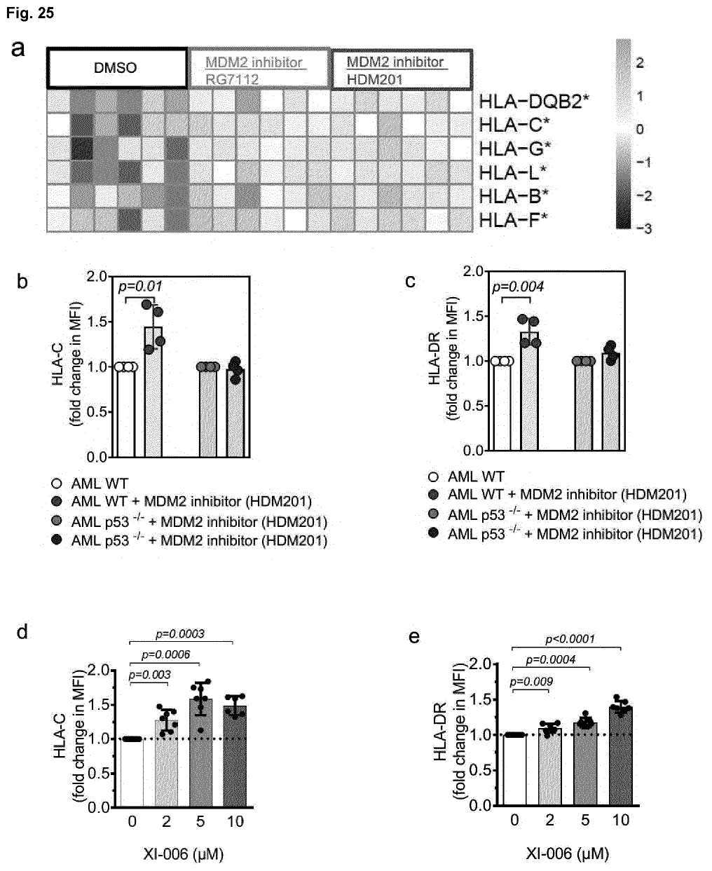

FIG. 25: MDM2 and MDMX inhibition upregulate MHC class I and II molecules.

(a) Microarray-based analysis of the expression level of HLA class I and II in OCI-AML3 cells after treatment with DMSO, RG-7112 (1 μM) or HDM-201 (200 nM) for 24 hours is shown as tile display from Robust Multichip Average (RMA) signal values, n=6 biologically independent samples per group.

(b, c) The graph shows fold-change of MFI for HLA-C (b), HLA-DR (c) expression on wildtype OCI-AML3 (p53+/+) or p53 knockout (p53−/−) OCI-AML3 cells after in vitro treatment with the indicated concentrations of MDM2-inhibitor HDM201 for 72 hours as mean±SEM from n=4 independent experiments. MFI of control-treated cells was set as 1.0. P-values were calculated using the two-sided Student's unpaired t-test.

(d, e) The graph shows fold-change of MFI for HLA-C (d) and HLA-DR (e) expression on OCI-AML3 cells after treatment with the indicated concentrations of MDMX-inhibitor XI-006 for 72 hours as mean±SEM from n=7 independent experiments. MFI of control-treated cells was set as 1.0. P-values were calculated using the two-sided Student's unpaired t-test.

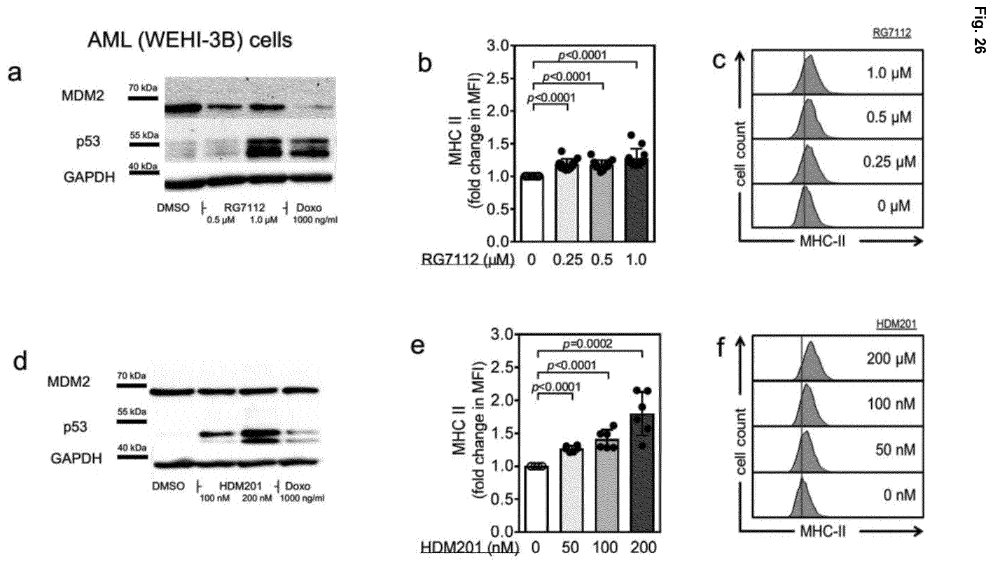

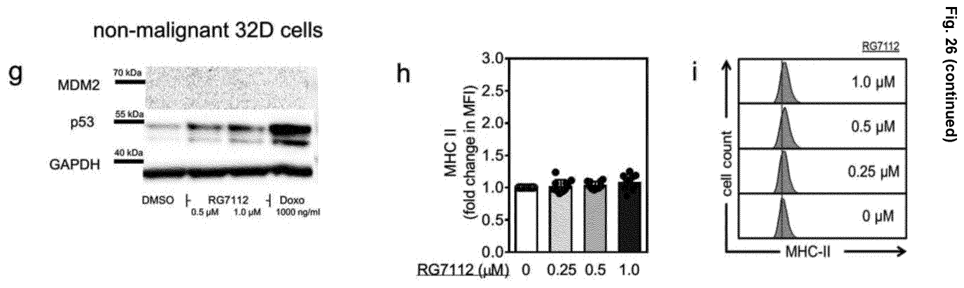

FIG. 26: MDM2 inhibition increases p53 and MHC class II expression in malignant WEHI-3B but not in non-malignant 32D cells.

(a) Western blot shows the expression of MDM2, p53 and loading control (GAPDH) in WEHI-3B cells exposed to DMSO, RG-7112 (0.5 μM, 1 μM) or 1000 ng/ml doxorubicin for 4 hours.

(b) The graph shows fold-change of MFI for MHC class II expression on WEHI-3B cells after treatment with the indicated concentrations of MDM2-inhibitor RG-7112 for 72 hours as mean±SEM from n=6 independent experiments. MFI of control-treated cells was set as 1.0. P-values were calculated using the two-sided Student's unpaired t-test.

(c) A representative flow cytometry histogram depicts the mean fluorescence intensity (MFI) for MHC class II expression on WEHI-3B cells after treatment with the indicated concentrations of MDM2-inhibitor RG-7112 for 72 hours.

(d) Western blot shows the expression of MDM2, p53 and loading control (GAPDH) in WEHI-3B cells exposed to DMSO, HDM201 (100 nM, 200 nM) or 1000 ng/ml doxorubicin for 4 hours.

(e) The graph shows fold-change of MFI for MHC class II expression on WEHI-3B cells after treatment with the indicated concentrations of MDM2-inhibitor HDM201 for 72 hours as mean±SEM from n=4-6 independent experiments. MFI of control-treated cells was set as 1.0. P-values were calculated using the two-sided Student's unpaired t-test.

(f) A representative flow cytometry histogram depicts the mean fluorescence intensity (MFI) for MHC class II expression on WEHI-3B cells after treatment with the indicated concentrations of MDM2-inhibitor HDM201 for 72 hours.

(g) Western blot shows the expression of MDM2, p53 and loading control (GAPDH) in 32D cells exposed to DMSO, HDM201 (100 nM, 200 nM) or 1000 ng/ml doxorubicin for 4 hours.

(h) The graph shows fold change of MFI for MHC class II expression on 32D cells after treatment with the indicated concentrations of MDM2-inhibitor HDM201 for 72 hours as mean±SEM from n=4-6 independent experiments. MFI of control-treated cells was set as 1.0. P-values were calculated using the two-sided Student's unpaired t-test.

(i) A representative flow cytometry histogram depicts the mean fluorescence intensity (MFI) for MHC class II expression on 32D cells after treatment with the indicated concentrations of MDM2-inhibitor HDM201 for 72 hours.

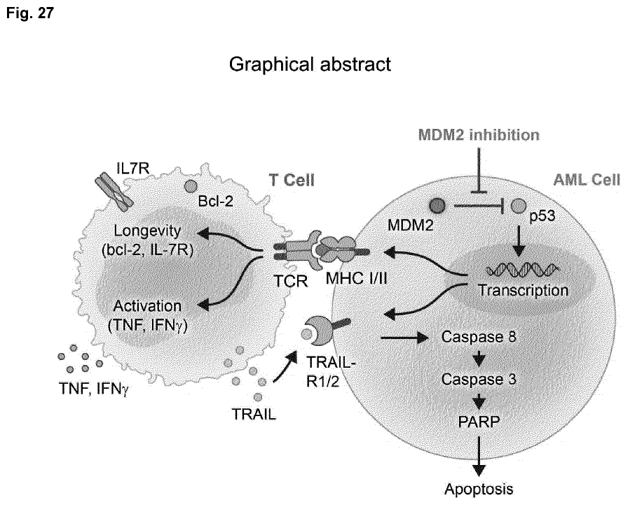

FIG. 27: Graphical abstract

Simplified sketch showing the proposed mechanism of action of MDM2 induced immune sensitivity of AML cells to T cells. MDM2-inhibition increases p53 levels. P53 translocates to the nucleus where it activates the transcription of MHC class I and II, as well as TRAIL-R1/2. Increased MHC II expression leads to T cell priming, thereby promoting their longevity and activation with consecutive cytokine production. TRAIL-R upregulation on the AML cells increases their sensitivity to TRAIL-mediated apoptosis induction by T cells, causing activation of the TRAIL-R1/2 downstream pathway (caspase-8, caspase-3, PARP) in AML cells.

### EXAMPLES

The invention is further described by the following examples. These are not intended to limit the scope of the invention but represent preferred embodiments of aspects of the invention provided for greater illustration of the invention described herein.

### Methods Employed in the Examples

### Isolation and Culture of Patient-Derived Peripheral Blood Mononuclear Cells (PBMCs)

Human sample collection and analysis were approved by the Institutional Ethics Review Board of the Medical center, University of Freiburg, Germany (protocol number 100/20). Written informed consent was obtained from each patient. All analysis of human data was carried out in compliance with relevant ethical regulations. The characteristics of patients are listed in Table 1.

### Isolation of human Peripheral Blood Mononuclear Cells (PBMC)

Human peripheral blood was collected in a sterile EDTA coated S-Monovette (Sarstedt, Germany). The blood was diluted 1:1 with PBS and layed over one volume of Pancoll Human (PAN-Biotech, Germany). Gradient centrifugation was conducted at 300×g without brake (acceleration: 9, deceleration: 1) for 30 minutes at room temperature to separate PBMC. The interphase containing the separated PBMC was aspirated and washed three times with PBS; once at 300×g, then twice at 200×g for 10 minutes.

### Isolation of CD4⁺ T Cells from Human PBMC

PBMC isolation was performed as described above. CD4⁺ T cells were enriched using the MACS cell separation system (Order no. 130-045-101 Miltenyi Biotec, USA) according to the manufacturer's instructions. For positive selection, anti-human CD4⁺ microBeads (Miltenyi Biotec, USA) were used. CD4⁺ T cell purity was at least 90% as assessed by flow cytometry.

### Primary Healthy Donor PBMC and Primary AML Blasts

Primary cells were maintained in RPMI media supplemented with 20% fetal calf serum, 2mM L-glutamine and 100 U/ml penicillin/streptomycin.

### Exposure of Primary AML Blasts to MDM2 Inhibition

PBMCs were isolated from AML patients' blood by Ficoll gradient centrifugation, according to the manufacturer's protocol (Sigma-Aldrich), plated in 24-well plates at a density of 500,000 cells per well and cultured for 48 h in RPMI-medium (Invitrogen, Germany) supplemented with 10% Fetal Calf Serum (FCS) in the presence or absence of RG-7112 (Selleck Chemicals Llc, USA) or HDM-201 (Novartis, Basel, Switzerland) at the concentrations indicated at the individual experiment.

### T Cell Activation and Cytotoxicity Assays

Cytotoxic T cells used in cytotoxicity assays were generated from peripheral blood T cells of healthy volunteer donors after isolation of donor blood by Ficoll gradient centrifugation, enriched by negative selection using Pan T Cell Isolation Kit II (Miltenyi Biotech) and the MACS cell separation system (Miltenyi Biotec) according to the manufacturer's instructions. Obtained T cell purity was at least 90% as assessed by flow cytometry. Isolated CD3⁺ T cells were stimulated with 25 μl Dynabeads™ Human T-Activator CD3/CD28 (Gibco, Thermo Fisher Scientific) per one million T cells at day 1 and with human Interleukin-2 (IL-2) at 30 U/ml (PeproTech) at day 2 after isolation and cultured for 7 days in total.

### Quantitative Real-Time PCR of Human AML Samples

Total RNA of isolated patient PBMCs, was isolated using the Qiagen Rneasy kit, according to manufacturer's instructions. The PBMCs were plated in 6-well plates at a density of ten million cells per well, cultured in RPMI-medium (Invitrogen) supplemented with 10% Fetal Calf Serum and treated with RG-7112 (0.5 μM, 1 μM and 2 μM) for 12 hours. For cDNA synthesis, 1 μg RNA was reverse-transcribed using random hexamer primers (Highcapacity cDNA reverse transcription kit applied Biosystems/ThermoFisher Scientific) and MultiScribe reverse transcriptase (ThermoFisher Scientific). Quantitative RT-PCR was performed using SYBR Green Gene expression Master Mix (Roche LightCycler 480 SYBR Green I Master) and primers as provided in Table 2. All reactions were performed with 50 ng cDNA in triplicates, correction and reproducibility measurements in duplicates and the relatives expression was calculated using the Pfaffl ΔCt method with all mRNA levels normalized to the reference gene hGAPDH. Primer sequences are provided in Table 2.

### Mice

C57BL/6 (H-2Kb) and BALB/c (H-2Kd) mice were purchased from Janvier Labs (France) or from the local stock at the animal facility of Freiburg University Medical Center. Rag2^{-/-}II2rγ^{-/-} mice were obtained from the local stock at the animal facility of Freiburg University Medical Center. Mice were used between 6 and 14 weeks of age, and only female or male donor/recipient pairs were used. Animal protocols were approved by the animal ethics committee Regierungsprasidium Freiburg, Freiburg, Germany (protocol numbers: G17-093, G-20/96).

### Graft-versus-leukemia (GvL) Mouse Models

GvL experiments were performed as previously described (5). Briefly, recipients were injected intravenously (i.v.) with leukemia cells +/− donor BM cells after (sub-) lethal irradiation using a ¹³⁷Cs source. CD3⁺ T-cells were isolated from donor spleens or peripheral blood of healthy donors and enriched by negative selection using Pan T Cell Isolation Kit II (Miltenyi Biotech, USA) and the MACS cell separation system (Miltenyi Biotec) according to the manufacturer's instructions. Obtained T-cell purity was at least 90% as assessed by flow cytometry. CD3⁺ T-cells were given on day 2 after BM transplantation.

### AML^{MLL-PTD FLT3-ITD} Leukemia Model

For the AML^{MLL-PTD FLT3-ITD} leukemia model, C57BL/6 recipients were transplanted with 5,000 AML^{MLL-PTD FLT3-ITD} cells and 5 million BALB/c BM cells i.v. after lethal irradiation with 12 Gy in two equally split doses performed four hours apart. A total of 300,000 BALB/c (allogeneic model) splenic CD3⁺ T cells were introduced i.v. on day 2 following initial transplantation as previously reported (19, 20).

### WEHI-3B Leukemia Model

For the WEHI-3B leukemia model, BALB/c recipients were transplanted with 5,000 AML (WEHI-3B) cells and 5 million C57/BL6 BM cells i.v. after lethal irradiation with 10 Gy in two equally split doses performed four hours apart. A total of 200,000 C57/BL6 (allogeneic model) splenic CD3⁺ T cells were introduced i.v. on day 2 following initial transplantation.

### OCI-AML3 Xenograft Model

For the OCI-AML3 xenograft model⁴ Rag2^{-/-}II2rγ^{-/-} recipients were transplanted with 200,000 OCI-AML3 (wildtype or TRAIL-R2 knockout) or one million OCI-AML3 (wildtype or p53 deficient) cells as indicated i.v. after sublethal irradiation with 5 Gy. A total of 500,000 human CD3⁺ T cells isolated from peripheral blood of healthy donors were introduced i.v. on day 2 following initial transplantation.

### Primary Human AML Xenograft Model

For the Primary human AML xenograft model (21) Rag2^{-/-}II2rγ^{-/-} recipients were used. Primary human AML cells were isolated by FICOLL density centrifugation and depleted from CD3⁺ cells by magnetic separation. Ten million CD3⁺ depleted primary human AML cells were transplanted i.v. after sublethal irradiation with 5 Gy. A total of 50,000 human CD3⁺ T cells isolated from peripheral blood of healthy donors were introduced i.v. on day 2 following initial transplantation.

### Leukemia Models Based on Oncogenic Mutations Introduced in the BM

To induce leukemia based on a certain oncogenic mutation, BALB/c recipients were transplanted with 30,000 BALB/c derived BM cells transduced with cKIT-D816V or FIP1L1-PDGFR-α. To induce the GVL effect the mice underwent irradiation with 10 Gy in two equally split doses performed four hours apart. The recipient mice where then injected with five million C57/BL6 BM cells i.v.; 200,000 C57/BL6 splenic T cells were introduced i.v. on day 2 following allogeneic BM transfer. Spleen derived T cells were enriched by depleting all cells other than CD3 positive cells by MACS.

### Drug Treatment in the Mouse Models

At day 3-11 after transplantation mice were treated every second day (5 doses) with RG-7112 (100 mg/kg) or vehicle (corn oil plus 5% DMSO) via oral gavage. At day 4 and 8 after transplantation purified anti-mouse CD253 (TRAIL) antibody or isotype control antibody were injected i.p. at a dose of 12.5 μg/g bodyweight when indicated in the respective experiment.

### T Cell Phenotyping in the GvL Mouse Model

T cell phenotyping experiments were performed using the WEHI-3B leukemia model. At day 12 following WEHI-3B i.v. injection, FACS analysis of spleens was performed.

### Leukemia Cell Lines

The following leukemia cell lines were used: AML^{MLL-PTD FLT3-ITD} (22) (murine), WEHI-3B (23) (murine) and OCI-AML3 (human). AML^{MLL-PTD FLT3-ITD} leukemic cells were provided by Dr. B. R.

Blazar (University of Minnesota). All cell lines used for in vivo experiments were authenticated at DSMZ or Multiplexion, Germany. All cell lines were tested repeatedly for Mycoplasma contamination and were found to be negative.

### Knockdown of p53 in OCI-AML3 Cells

P53 knockdown cells have been previously described (24). The p53 shRNA (p53.1224) had been cloned into a retroviral vector that co-expressed red fluorescent protein and which could be induced by doxycycline (24). Transfected cells were cultured in 20% FCS RPMI media containing 1 μg/ml doxycycline and 50 μg/ml blasticidin for stable knockdown efficiencies. The knockdown of p53 was confirmed by Western blotting.

### Knockdown of TRAIL R1/R2 in OCI-AML3 Cells

HEK293T packaging cells were cultured in DMEM medium (Invitrogen, Germany) supplemented with 10% Fetal Calf Serum (FCS). Chloramphenicol-resistant lentiviral vectors, pGFP-C-shLenti human TRAIL-R1-targeted shRNA (clone ID: TL308741A 5′-TTCGTCTCTGAGCAGCAAATGGAAAGCCA-3′ (SEQ ID NO: 13)), pGFP-C-shLenti human TRAIL-R2-targeted shRNA (clone ID: TL300915B 5′-AGAGACTTGCCAAGCAGAAGATTGAGGAC-3′ (SEQ ID NO: 14)) and pGFP-C-shLenti non-silencing shRNA control (clone ID: TR30021[AM1] 5′-GCACTACCAGAGCTAACTCAGATAGTACT-3′ (SEQ ID NO: 15)), were purchased from OriGene, USA. Lentiviral particles were generated by transfection of HEK293T cells using Lipofectamine 2000. 300,000 OCI-AML3 cells were transduced with the lentiviral particles in the presence of 4 μg/μl Polybrene (Merckmillipore). Knockdown of TRAIL-R1 and TRAIL-R2 was confirmed by FACS analysis.

### Generation of TRAIL-R2 Knockout OCI-AML3 Cells

The Neon Transfection System (Invitrogen) was used to deliver a CRISPR-Cas9 system that expresses the gRNA, Cas9 protein and puromycin resistance gene (PMID: 25075903). TRAIL-R2 gRNA design (5′-CGCGGCGACAACGAGCACAA-3′ (SEQ ID NO: 16)) and cloning into the lentiCRISPR v2 vector (Addgene plasmid #52961) was performed according to Zhang lab protocols as previously described (PMID: 31114586). To deliver the lentiCRISPR v2-TRAIL-R2 plasmid, 200.000 OCI-AML3 cells were resuspended in resuspension buffer R (Neon Transfection System, Invitrogen) in presence of 2 μg plasmid. Cells were electroporated using the

Neon Transfection System in 10 μl Neon tips at 1350 V, 35 ms, single pulse and immediately transferred to antibiotic-free recovery medium. TRAIL-R2 negative cells were isolated by cell sorting (BD Aria Fusion) and verified by flow cytometric analysis.

### Isolation of Mouse Splenic Cells and PMA/Ionomycin Stimulation

Single cell suspensions were obtain by mashing the spleens through 70 mm cell strainers. Red blood cells were lysed 2 minutes on ice with 1 mL of 1×RBC Lysis Buffer (ThermoFisher), samples were washed with PBS and centrifuged for 7 min at 400 g. Cells were re-stimulated in 2 ml RPMI supplemented with Golgi-Stop and Golgi-Plug (1:1000, BD), phorbol 12-myristate 13-acetate (50 ng/ml, Applichem) and lonomycin (500 ng/ml, Invitrogen) for 5 hours at 37° C.

### Microarray Analysis

Total RNA from OCI-AML3 cells was extracted at 24 hrs after treatment with the MDM2 inhibitors RG-7112 (2 μM) or HDM-201 (500 nM) using miRNeasy Mini kit (Qiagen, Netherlands) and DNase (Qiagen, Germany) according to manufacturer's instructions. RNA integrity was analyzed by capillary electrophoresis using a Fragment Analyser (Advanced Analytical Technologies, Inc. Ames, IA). RNA samples were further processed with the Affymetrix GeneChip Pico kit and hybridized to Affymetrix Clariom S arrays as described by the manufacturer (Affymetrix, USA). The arrays were normalized via robust multichip averaging as implemented in the R/Bioconductor oligo package. Gene set enrichment was calculated using the R/Bioconductor package ‘gage’48 using the pathways from the ConsensusPathDB 49 as gene sets and a significance cutoff p<0.05.

Microarray analysis was performed as previously described (26). Microarray data are deposited in the database GEO repository under the GEO accession GSE158103.

### Western Blotting

OCI-AML3 cells were cultured in the presence or absence of 1 mg/ml Doxorubicin (pharmacy of

Freiburg University Medical Center) or 1 μM RG-7112 (Selleck Chemicals Ltc) for 4 h and total protein extracts were prepared as described previously (27). To detect caspase activation, OCI-AML3 cells were treated with 1 μM RG-7112 for 72 h and were co-cultured with activated T cells at the effector-to-target (E:T) ratio of 10:1 for 4 h. In some experiments, T cells were incubated with neutralizing antibody against TRAIL (10 μg/ml, MAB375, R&D Systems) or mouse IgG1 (#401408, BioLegend) 1 h prior to coculture. After T cells were removed by using Pan T Cell Isolation Kit II, OCI-AML3 cells were subjected to analysis.

Primary murine bone marrow cells transduced with EV (empty vector), FLT3-ITD, KRAS-G12D, cKIT-D816V, JAK2-V617F, FIP1L1-PDGFR-α, BCR-ABL or c-myc were sorted for GFP expressing cells using a BD FACSAria III cell sorter (BD Bioscience, Germany) and subjected to analysis.

Cells were lysed in radioimmunoprecipitation assay (RIPA) buffer (Santa Cruz Biotechnology) supplemented with Phosphatase Inhibitor Cocktail 2 (Sigma-Aldrich) and protein concentrations were determined using the Pierce BCA Protein Assay Kit (Life Technologies). Cell lysates prepared for SDS-PAGE using NuPAGE™ LDS sample buffer and NuPAGE™ sample reducing agent

(Invitrogen). Supernatant samples from cell-free supernatants were prepared using sample buffer containing SDS and Dithiothreitol (DTT). The primary antibodies were used against p53 (#2527, Cell Signaling Technology), MDM2 (#86934, Cell Signaling Technology), Caspase-3 (#9662, Cell Signaling Technology). Anti-GAPDH (#GAPDH-71.1, Sigma-Aldrich) and anti-β-Actin (#4970, Cell Signaling Technology) were used as internal loading control. As a secondary antibody, horseradish peroxidase (HRP)-linked anti-rabbit or anti-mouse IgG were used (#7074, #7076, Cell Signaling Technology). The blot signals were detected using WesternBright Quantum or Sirius HRP substrate (Advansta), imaged using ChemoCam Imager 3.2.0 (Intas Science Imaging Instruments GmbH) and quantified using ImageJ (NIH) software.

### Row Cytometry

All antibodies used for flow cytometry analyses are listed in Table 3. For excluding dead cells, the LIVE/DEAD Fixable Dead Cell Stain kit (Molecular Probes, USA) or LIVE/DEAD™ Fixable Aqua Dead Cell Stain Kit (Thermo Scientific) along with True Stain FcX (BioLegend) were used, according to the manufacturer's instructions. For all flurochrome-conjugated antibodies, optimal concentrations were determined using titration experiments. Cells were incubated with the respective antibodies diluted in FACS buffer for 20 minutes at 4° C. for surface antigen staining. Cells were then washed with FACS buffer according to the manufacturer's instruction. For mouse Bcl-2 analysis, cells were fixed with one part prewarmed 3.7% formalin and one part FACS buffer and were then incubated in 90% methanol for 30 minutes before the Bcl-2 antibody was added. Intracellular cytokine staining was performed using the BD Cytofix/Cytoperm kit (BD Biosciences, Germany) or the Foxp3/Transcription Factor Staining Buffer Set (ThermoFisher) according to the manufacture's instruction. For intracellular cytokine staining of mouse IFN-γ, before staining, cells were restimulated according to manufacturer's instructions with dilution of Cell Stimulation Cocktail (eBioscience, Germany) containing PMA and ionomycin for 4 hours. Data were acquired on the BD LSR Fortessa flow cytometer (BD Biosciences, Germany) and analyzed using Flow Jo software version 10.4 (Tree Star, USA). For high dimensional analysis, data were acquired on Cytek Aurora (Cytek Biosciences) and pre-processed using Flow Jo software version 10.4 (Tree Star, USA) for singlets and dead cell exclusion and CD45 positive cell selection.

### Algorithm-Guided High-Dimensional Analysis of Spectral Flow Cytometry Data

High-dimensional analysis was performed in the R environment. Two-dimensional UMAPs (Uniform Manifold Approximation and Projections) were generated using the umap package and the FIowSOM-based metaclustering was performed as described by Brumelman et al. (25).

### Killing Assay

OCI-AML3 target cells were cultured in 20% FCS-supplemented RMPI medium in the presence or absence of 1 μM RG-7112 for 72 h, labeled with 0.5 mM Cell Trace Violet BV421 (Thermo Fisher Scientific, Germany) according to manufacturer's instructions and co-cultured with effector T cells at a effector to target ratio of 10:1, 5:1, 2:1 and 1:1 for 16 h in 96-well plates. Cytotoxicity of effector T cells was measured using Zombie NIR APC/Cy7 (Biolegend).

For Killing assays using recombinant hTRAIL ((TNFSF 10, Apo-2L, CD253;) SUPERKILLERTRAIL®; ENZO), the ligand was added for 24 h 0.5 μg/ml (1:1000) for optimal killing and 0.25 μg/ml (1:2000) for limiting killing conditions to OCI-AML3 target cells. Viability of cells was assessed by LIVE/DEAD™ Fixable Aqua Dead Cell Stain Kit (Thermo Scientific). Data were acquired on the BD LSR Fortessa flow cytometer (BD Biosciences) and analyzed using Flow Jo software version 10.4 (Tree Star).

### Chromatin Immunoprecipitation (ChIP Assay)

OCI-AML3 cells were treated with 2 μM RG-7112 for 12 h and were crosslinked with 1% formaldehyde for 10 min at room temperature, and formaldehyde was inactivated by the addition of glycine to a final concentration of 125 mM. Cells were resuspended with lysis buffer (1% SDS, 10 mM EDTA, 50 mM Tris-Cl, pH 8.0, protease inhibitor cocktail) and sonicated for 15 min in a Bioruptor using a 30 sec on/off program at high power. After centrifugation at 16,000 g for 5 min, the supernatant was collected and diluted 10-fold with dilution buffer (20 mM Tris-Cl, pH 8.0, 2 mM EDTA, 150 mM NaCl, 1% Triton X-100, protease inhibitor cocktail). Prepared chromatin extracts were incubated with mouse IgG (sc-2025, Santa-Cruz Biotechnology) or anti-p53 antibodies (sc-126, Santa-Cruz Biotechnology) overnight at 4° C. Immune complexes were collected using Dynabeads Protein G (Invitrogen) beads for 2 h on a rotator at 4° C., washed 5 times with wash buffer (20 mM Tris-Cl, pH 8.0, 2 mM EDTA, 0.1% SDS, 0.5% NP-40, 0.5 M NaCl, protease inhibitor cocktail) and 4 times with TE buffer (10 mM Tris-Cl, pH 8.0, 1 mM EDTA). DNA was eluted for 6 h at 65° C. in elution buffer (100 mM NaHCO3, 1% SDS) and purified by using QlAquick Gel extraction Kit. Quantitative PCR was used to measure enrichment of bound DNA and was carried out using the LightCycler 480 SYBR Green I Master kit (Roche, Switzerland) in a LightCyler 480 instrument (Roche, Switzerland). Primer sequences are provided in Table 2.

ChIP-qPCR data for each primer pair are represented as percent input by calculating amounts of each specific DNA fragment in immunoprecipitates relative to the quantity of that fragment in input DNA.

### Tumor Cell Lines

The human leukemia cell lines OCI-AML3, MOLM-13, the murine leukemia cell line WEHI-3B and non-malignant 32D cells were purchased from ATCC (American Type Culture Collection, Manassas, Virginia, USA) and cultured in RPMI media supplemented with 10% FCS, 2 mM L-glutamine and 100 U/ml penicillin/streptomycin.

### Recall Immunity Experiment

For the GvL recall immunity experiment, splenocytes were harvested from C57BL/6 BMT recipients (5 million BALB/c BM and 5,000 AML^{MLL-PTD/FLT3-ITD} cells (d0), 300,000 allogeneic T cells (d2)) on day 12 after allo-HCT. FACS sorting for donor H-2kb⁺CD3⁺CD8⁺ T cells was then performed. Cell purity was at least 90% as assessed by flow cytometry. We transplanted 100,000 sorted cells i.v. to secondary recipients on day 2 following 5 million BALB/c BM and 5,000 AML^{MLL-PTD/FLT3-ITD} cell injection (d0).

### Depletion of NK Cells in Murine Bone Marrow

To deplete NK cells, naive BALB/c BM was isolated and stained for CD3 and NK1.1 surface. Through FACS Sorting, BM was then excluded of NK1.1⁺CD3⁻ cells resulting in the depletion of NK cells in the BM.

### Depletion of CD8⁺ T Cells in Murine Bone Marrow

To deplete CD8⁺ T cells, extracted BM was stained for CD3 and CD8 surface markers. In this case, BM was excluded of CD3⁺ CD8⁺ cells through FACS sorting generating BM depleted of CD8⁺ T cells.

### GVHD Histology Scoring

GVHD scoring was performed as previously described (28). The organs small intestines, large intestines and liver were isolated and tissue sections were H&E stained and evaluated a by a pathologist blinded to the treatment groups.

### Extracellular Flux Assay

Extracellular flux assays were performed on a Seahorse analyzer (Agilent) as recommended by the manufacturer. Briefly, 200 000 T-cells were plated in each well of a 96-well Seahorse XF Cell Culture Microplate in Seahorse XF Base Medium supplemented with 2 mM glutamine. The cell culture plate was then incubated for 45 min in a 37° C. non-CO₂ incubator. Sensor cartridge ports were loaded with glucose, oligomycin and 2-deoxyglucose (2-DG). Glycolysis stress test was performed by measuring basal extracellular acidification rate (ECAR) followed by sequential injections of glucose (final concentration 10 mM), oligomycin (final concentration 1 μM) and 2-DG (final concentration 50 mM).

### Transfection of Primary Mouse BM Cells with Common Oncogenic Mutations or Gene Fusions

To generate EV-tg, FLT3-ITD-tg, KRASG12V-tg, cKITD816V-tg, JAK2V617F-tg, FIP1L1-PDGFRα-tg, BCRabl-tg, cMYC-tg BM cells BALB/c mice were injected with 100 mg/kg 5-fluorouracil (Medac GmbH) four days prior to bone marrow harvest. Murine bone marrow was collected and prestimulated overnight with growth factors (10 ng/mL mIL-3, 10 ng/mL mIL-6 and 14.3 ng/mL mSCF) as described previously by us (5, 29). Cell were transduced by 3 rounds of spin infection (2400 rpm, 90 min, 32° C.) every 12 hours by adding 2 mL retroviral supernatant supplemented with growth factors and 4 μg/mL polybrene.

### Sample Preparation for Mass Spectrometry

CD8⁺ T cells were enriched from the spleens of recipient mice on day 12 after allo-HCT. T cells were incubated at a cell density of 2,000,000 cells/ml in RPMI 1640 medium supplemented with 10% fetal calf serum (Gibco), 4 mM L-glutamine, 100 I.U./ml peniciliin, 100 μg/ml streptomycin, 100 U/ml human recombinant IL-2, and 55 μM beta-mercaptoethanol for 90 minutes at 37° C. After that, the cells were washed with PBS and the medium was exchanged with glucose-free RPMI 1640 medium, supplemented as above with addition of 10 mM U-¹³C-glucose. Labeling with U-¹³C-glucose was performed for 50 minutes. One million cells per sample were harvested and separated from the cell culture medium by centrifugation at 500 g for 5 minutes. at 4° C. Cells were washed with 500 μl PBS, followed by another centrifugation step at 500 g for 5 min at 4° C. After complete removal of the supernatant, metabolites were extracted by resuspending the cell pellet in 50 μl methanol:acetonitrile:water (50:30:20) buffer pre-chilled on dry ice for 30 minutes. Samples were vortexed briefly and stored at −80° C.

### Liquid Chromatography-Mass Spectrometry (LC-MS)

LC-MS was carried out using an Agilent 1290 Infinity II UHPLC in line with a Bruker Impact II QTOF-MS operating in negative ion mode. Scan range was from 20 to 1050 Da. Mass calibration was performed at the beginning of each run. LC separation was on a Hilicon iHILIC(P) classic column (100×2.1 mm, 5 μm particles) using a solvent gradient of 95% buffer B (90:10 acetonitrile:buffer A) to 20% buffer A (20 mM ammonium carbonate+5 μM medronic acid in water). Flow rate was 150 μL/min. Autosampler temperature was 5 degrees and injection volume was 2 μL. Data processing for targeted analysis of the absolute abundance of metabolites was performed using the TASQ software (Bruker). Peak areas for each metabolite were determined by manual peak integration. Only metabolite peaks that were detected in >80% of the samples were further analyzed. Missing values were calculated as 50% of the lowest value detected in the whole sample set for this metabolite. Statistical comparisons were performed using the unpaired two-sided Student's t-test. Heatmaps were generated using MetaboAnalyst 5.0 (30) as follows: peak area values were subjected to logarithmic transformation and auto-scaling; metabolites were clustered using hierarchiral clustering with Ward agglomeration method on Euclidian distance. Data processing for ¹³C-glucose tracing, including correction for natural isotope abundance, was performed as described previously (31, 32).

### Statistical Analysis

For the sample size in the murine GVL survival experiments a power analysis was performed. A sample size of at least n=10 per group was determined by 80% power to reach a statistical significance of 0.05 to detect an effect size of at least 1.06. Differences in animal survival (Kaplan-Meier survival curves) were analyzed by Mantel Cox test. The experiments were performed in a non-blinded fashion. For statistical analysis an unpaired t-test (two-sided) was applied. Data are presented as mean and SEM. (error bars). Differences were considered significant when the P-value was <0.01.

### Tables of the Examples

TABLE 1
AML patient characteristics

| | | | % Blast | % Blast | | Status at time |
|---|---|---|---|---|---|---|
| | | Cytogenetics | Count | Count | Blast | point of |
| Patient | Gender | Molecular markers | PB | BM | phenotype | analysis |
| 1 | f | 21q22/RUNX1 | 13 | not | CD34⁺ | first diagnosis |
| | | mutation; Monosomy 7 | | available | CD117⁺ | |
| 2 | m | FLT3-ITD mutation; | 7 | not | CD33⁺ | Pretreated with |
| | | NPM1 mutation | | available | D117⁺ | Midostaurin |
| 3 | m | Deletion 17p13 (TP53); | not | not | CD19⁺ | Excluded |
| | | Deletion 11q22(ATM) | available | available | CD20⁺ | because B cell |
| | | | | | | malignancy |
| 4 | m | NMP1 mutation | 42 | 71 | CD33⁺ | first diagnosis |
| 5 | | | | | | |
| 6 | f | Deletion 5q31/5q33 | 40 | 20 | CD34⁺ | first diagnosis |
| | | (EGR1)/RPS14 | | | CD117⁺ | |
| 7 | m | | | | CD34⁺ | |
| 8 | F | Monosomy 7; | 30 | 14 | CD34⁺ | first diagnosis |
| | | Monosomy 16 | | | CD117⁺ | |
| 9 | F | FLT3-ITD mutation; | 45 | 35 | CD117⁺ | first diagnosis |
| | | NRAS mutation | | | | |
| 10 | f | DNMT3A; IDH1; NPM1; | 94 | 99 | CD33⁺ | first diagnosis |
| | | PHF6 mutation | | | | |
| 11 | m | BCOR, CBL; RUNX1; | 20 | 43 | CD34⁺ | first diagnosis |
| | | STAG2 mutation; | | | | |
| | | Trisomie 8 | | | | |
| 12 | m | NPM1; JAK2 V617F | 4 | 57 | CD117⁺ | first diagnosis |
| | | Mutation | | | | |
| 13 | m | NRAS mutation | 96 | not | CD34⁺ | Relapse post- |
| | | | | available | | allo-HCT |
| 14 | f | RUNX1 mutation | 14 | not | not | first diagnosis |
| | | | | available | detectable | SAML (from |
| | | | | | | MDS) |
| 15 | F | t(9;22)/BCR-ABL1 | 21 | not | CD34⁺ | first diagnosis |
| | | translocation; TP53; | | available | CD117⁺ | SAML (from |
| | | Tet2 mutation | | | | MDS) |
| 16 | F | FLT3-ITD; NPM1; | 38 | 90 | CD34⁺ | first diagnosis |
| | | DNMT3A; TET2 | | | CD117⁺ | |
| | | mutation | | | | |
| 17 | F | FLT3; PTPN11; NRAS; | 85 | 92 | CD117⁺ | first diagnosis |
| | | IDH2; NPM1; SRSF2 | | | | |
| | | mutation | | | | |
| 8 | F | EZH2; BCORL1; | 80 | 66 | CD34⁺ | first diagnosis |
| | | NRAS; TET2; STAG2 | | | CD117⁺ | SAML (from |
| | | mutation | | | CD33⁺ | MDS) |
| 19 | F | KRAS; NPM1; TET2 | 89 | 87 | CD14⁺ | first diagnosis |
| | | mutation | | | | SAML (from |
| | | | | | | CMML) |
| 20 | m | ASXL1; JAK2; RNX1; | 12 | not | CD34⁺ | first diagnosis |
| | | U2AF1; ZRSR2; | | available | | SAML (from |
| | | PTPN11; STAG2 | | | | MDS) |
| | | mutation | | | | |
| 21 | F | IDH2; NRAS; KRAS | 48 | 5 | CD34⁺ | first diagnosis |
| | | mutation | | | | SAML (from |
| | | | | | | MDS) |
| 22 | F | NOTCH1; NRAS; TP53 | 54 | not | CD34⁺ | first diagnosis |
| | | mutation; Trisomy 8; | | available | CD117⁺ | |
| | | Trisomy 11 | | | | |
| 23 | F | none | 57 | 81 | CD117⁺ | first diagnosis |
| 24 | m | Genotype: XXYY; | 9 | 52 | CD34⁺ | first diagnosis |
| | | EZH2; CEBPA | | | CD117⁺ | |
| | | mutation | | | | |
| 25 | m | not available | 92 | 90 | CD117⁺ | first diagnosis |
| 26 | m | Trisomy 8; Trisomy 11 | 56 | not | CD34⁺ | first diagnosis |
| | | | | available | CD117⁺ | |
| 27 | m | RUNX1-RUNX1T1 | 59 | 74 | CD34⁺ | first diagnosis |
| | | mutation; Trisomy 8; | | | | |
| | | Chr. Y deletion | | | | |
| 28 | m | FLT3; IDH2; NPM1; | 38 | 60 | CD117⁺ | first diagnosis |
| | | PTPN11 mutation | | | | |
| 29 | f | RUNX1T1; TP53; CBL | 35 | not | CD34⁺ | first diagnosis |
| | | mutation; Trisomy 8 | | available | | |
| 30 | f | EZH2; PTPN11; | 6 | 28 | CD117⁺ | first diagnosis |
| | | STAG2 mutation | | | | (AML/MDS) |
| 31 | m | Trisomy 8; FGFR1 | 14 | 15 | CD117⁺ | first diagnosis |
| | | (8p11)-rearrangement | | | | (AML/MDS) |
| 32 | F | JAK2 V617F; PTPN11 | 28 | not | CD34⁺ | first diagnosis |
| | | mutation | | available | | SAML (from |
| | | | | | | MDS) |
| 33 | m | ASXL1; SRSF2 | not | 21 | CD34⁺ | first diagnosis |
| | | mutation | available | | CD117⁺ | SAML (from |
| | | | | | | MDS) |
| 34 | F | Monosomy 7; Deletion | 29 | not | CD34⁺ | first diagnosis |
| | | 13q14; Chr 17p13 | | available | CD117⁺ | SAML (from |
| | | (TP53); ETV6-RUNX1; | | | | MPN) |
| | | JAK2 mutation | | | | |
| 35 | m | not available | 7 | not | not | first diagnosis |
| | | | | available | detectable | |
| 36 | m | BCOR; SF3B1; TET2 | 21 | not | CD34⁺ | first diagnosis |
| | | mutation | | available | CD117⁺ | |
| 37 | m | FLT3-ITD; IDH1 | 79 | 90 | CD33⁺ | first diagnosis |
| | | mutations | | | | |
| 38 | m | KMT2A (MLL) (11q23) | 97 | 28 | CD34⁺ | first diagnosis |
| | | rearrangement | | | CD33⁺ | |
| 39 | F | JAK2; TP53 mutation; | 25 | not | CD34⁺ | first diagnosis |
| | | Monosomy 17; Deletion | | available | CD117⁺ | SAML (from |
| | | 20g12 | | | | MDS) |
| 40 | m | Translocation | 21 | 2 | CD64⁺ | first diagnosis |
| | | t(15;17)/PML-RARA; | | | | |
| | | RARA817q21) | | | | |
| | | rearrangement | | | | |
| 41 | m | Monsomy 7; MECOM | 25 | 24 | CD34⁺ | first diagnosis |
| | | (3q26) rearrangement | | | | |
| 42 | F | Trisomy 8; IDH1; JAK2; | 94 | not | CD117⁺ | first diagnosis |
| | | RUNX1; SRSF2; TET2 | | available | | |
| | | mutation | | | | |
| 43 | m | U2AF1 mutation | not | not | CD34⁺ | Relapse |
| | | | available | available | CD117⁺ | |
| 44 | m | None | 12 | 71 | CD34⁺ | first diagnosis |
| | | | | | CD117⁺ | |
| 45 | F | IDH1 mutation | not | not | CD34⁺ | ALL/MM |
| | | | available | available | CD117⁺ | |
| 46 | m | ASXL1; DNMT3A; | 90 | 75 | CD34⁺ | first diagnosis |
| | | IDH1; PHF6; RUNX1 | | | CD117⁺ | |
| | | mutation | | | | |
| 47 | m | Trisomy 11; Trisomy 8; | 4 | 43 | CD34⁺ | first diagnosis |
| | | RUNX1T1 mutation | | | | SAML (from |
| | | | | | | MDS) |
| 48 | f | NPM1; IDH2 mutation | not | not | CD117⁺ | first diagnosis |
| | | | available | available | | |
| 49 | f | DNMT3A; RUNX1 | 32 | 50 | CD34⁺ | Relapse |
| | | mutation | | | | |
| 50 | m | DNMT3A; IDH1; | 77 | 93 | CD117⁺ | first diagnosis |
| | | SMC1A; TET2; | | | | |
| 51 | m | IDH2; IKZF1; NRAS; | 9 | 80 | CD34⁺ | first diagnosis |
| | | TET2 mutation | | | CD117⁺ | |
| 52 | f | DNMT3A; FLT2; | 25 | 1 | CD34⁺ | first diagnosis |
| | | KDM6A; NPM1; NRAS; | | | CD117⁺ | |
| | | SF3B1; TET2; WT1 | | | | |
| | | mutation | | | | |
| 53 | m | JAK2; RUNX1; SRSF2; | 96 | not | CD33⁺ | first diagnosis |
| | | TET2 mutation | | available | | |
| 54 | m | FLT3; NPM1; TET2 | 5 | not | CD33⁺ | |
| | | mutation | | available | CD117⁺ | |
| 55 | m | BCOR, FLT3 | 85 | 80 | CD34+ | first |
| | | mutation | | | | diagnosis |
| | | | | | | AML (from |
| | | | | | | MDS) |
| 56 | f | FLT3, IDH2, STAG2 | 70 | 73 | CD117+ | first |
| | | mutation | | | | diagnosis |
| 57 | m | DNMT3A, NPM1 | 57 | | CD117+ | first |
| | | (variant A), SRSF2, 2 | | | | diagnosis |
| | | TET2 mutations | | | | |

Abbreviations:
Pat. = patient,
f = female,
m = male,
sAML = secondary AML,
MDS = Myelodysplastic syndrome

TABLE 2
Primer sequences.

| Gene | forward | reverse |
|---|---|---|
| hTrailR1 | 5′- | 5′- CCTGGTTTGCACTGACATGCTG-3′ (SEQ ID NO: 2) |
| | GTGTGGGTTACACCAATGCTTC- | |
| | 3′ (SEQ ID NO: 1) | |
| hTrailR2 | 5′- | 5′- CCAGGTCGtTGTGAGCTTCT-3′ (SEQ ID NO: 4) |
| | ACAGTTGCAGCCGTAGTCTTG- | |
| | 3′ (SEQ ID NO: 3) | |
| CDKN1A | 5′ - | 5′- CTGAAAACAGGCAGCCCAAG-3′ (SEQ ID NO: 6) |
| | GTGGCTCTGATTGGCTTTCTG- | |
| | 3′ (SEQ ID NO: 5) | |
| TNFRSF10A | 5′- | 5′- AAGTGGCAAAACGACTCCGA-3′ (SEQ ID NO: 8) |
| | TTCGCATTCGGAGTTCAGGG-3′ | |
| | (SEQ ID NO: 7) | |
| TNFRSF10B | 5′- ACGACTGGTGCGTCTTGC-3′ | 5′- AAGACCCTTGTGCTCGTTGTC-3′ (SEQ ID NO: 10) |
| | (SEQ ID NO: 9) | |
| GAPDH | 5′- | 5′- ACCACCCTGTTGCTGTAGCCAA -3′ (SEQ ID NO: |
| | GTCTCCTCTGACTTCAACAGCG- | 12) |
| | 3′ (SEQ ID NO: 11) | |

TABLE 3
Flow cytometry antibodies.

| Antigen | Fluorochrome | Isotype | Clone | Dilution | Vendor |
|---|---|---|---|---|---|
| Anti-mouse Bcl-2 | PE-Cy7 | Mouse IgG1, κ | BCL/10C4 | 1:50 | BioLegend |
| Anti-human CD117 | PE | Mouse IgG1, κ | 104D2 | 1:50 | BioLegend |
| (c-kit) | | | | | |
| Anti-mouse CD3 | Pacific Blue | Rat IgG2b, κ | 17A2 | 1:100 | BioLegend |
| Anti-human CD34 | PE | Mouse IgG2a, κ | 561 | 1:50 | BioLegend |
| Anti-mouse CD40L | PerCP-Cy5.5 | Armenian hamster | MR1 | 1:100 | BioLegend |
| (CD154) | | IgG | | | |
| Anti-mouse CD45 | PerCP-Cy5.5 | Rat IgG2b, κ | 30-F11 | 1:100 | BioLegend |
| Anti-mouse CD8a | APC-H7 | Rat (LOU) IgG2a, | 53-6.7 | 1:50 | BD Pharmigen |
| | | κ | | | |
| Anti-mouse CD69 | APC | Armenian hamster | H1.2F3 | 1:100 | eBioscience |
| | | IgG | | | |
| Anti-mouse H-2kb | FITC | Mouse IgG2a, κ | AF6-88.5 | 1:100 | BioLegend |
| Anti-mouse H-2kb | APC | Mouse IgG2a, κ | AF6-88.5.5.3 | 1:50 | eBioscience |
| Anti-mouse H-2kd | Pacific Blue | Mouse (SJL) | SF1-1.1 | 1:50 | BioLegend |
| | | IgG2a, κ | | | |
| Anti-human HLA-A,B,C | APC | Mouse IgG2a, κ | W6/32 | 1:20 | BioLegend |
| Anti-human HLA-DR | Pacific Blue | Mouse IgG2a, κ | L243 | 1:50 | BioLegend |
| Anti-mouse IL-17a | PerCP-Cy5.5 | Rat IgG1, κ | TC11-18H10.1 | 1:50 | BioLegend |
| Anti-mouse IL-7Ra | PE | Rat IgG2a, κ | A7R34 | 1:100 | eBioscience |
| (CD127) | | | | | |
| Anti-mouse | PE | Mouse IgG1, κ | XMG1.2 | 1:100 | eBioscience |
| INF-γ | | | | | |
| Anti-mouse MHC Class II | PE-Cy7 | Rat IgG2b, κ | M5/114.15.2 | 1:50 | eBioscience |
| (I-A/I-E) | | | | | |
| p53 | FITC | Mouse IgG2b | DO-7 | 1:25 | BioLegend |
| Anti-mouse Perforin | APC | Rat IgG2a, κ | eBioOMAK-D | 1:50 | Invitrogen |
| Anti-human TRAIL-R1 | APC | Mouse IgG1 | 69036 | 1:20 | R&DSystems |
| Anti-human TRAIL-R2 | Alexa Fluor 488 | Mouse IgG2b | 71908 | 1:20 | R&DSystems |
| Anti-human TRAIL-R2 | PE | Mouse IgG2b | 71908 | 1:20 | R&DSystems |
| Anti-human TRAIL-R3 | PE | Mouse IgG1, κ | DJR3 | 1:30 | BioLegend |
| Anti-human TRAIL-R4 | PE | Mouse IgG1 | TRAIL-R4-01 | 1:10 | Invitrogen |
| (CD264) | | | | | |

Antibodies used for experiments for UMAP analysis

| | | | | | |
|---|---|---|---|---|---|
| Anti-mouse CD45 | BUV 395 | Rat IgG2b, κ | 30-F11 | 1:500 | BD |
| | | | | | Biosciences |
| Anti-mouse CD11b (Mac-1) | BUV 661 | Rat IgG2b, κ | M1/70 | 1:500 | BD |
| | | | | | Biosciences |
| Anti-mouse CD8a | BUV 805 | Rat IgG2a, κ | 53-6.7 | 1:100 | BD |
| | | | | | Biosciences |
| Anti-mouse TCR beta | PE-Cy5 | Armenian hamster | H57-597 | 1:300 | BioLegend |
| chain | | IgG | | | |
| Anti-mouse H-2Kb | BV 421 | Mouse IgG2a, κ | AF6-88.5 | 1:100 | BioLegend |
| Anti-mouse TIGIT | PE-Dazzle594 | Mouse IgG1, κ | 1G9 | 1:100 | BioLegend |
| (WUCAM, Vstm3) | | | | | |
| Anti-mouse CD73 | APC-Cy7 | Rat IgG1, κ | TY/11.8 | 1:200 | BioLegend |
| Anti-mouse CD279 (PD1) | BV 605 | Rat IgG2a, κ | 29F.1A12 | 1:100 | BioLegend |
| Anti-mouse CD127 (IL- | PE-Cy7 | Rat IgG2b, κ | SB/199 | 1:200 | BD |
| 7Ra) | | | | | Biosciences |
| Anti-mouse CD39 | PerCP-eFlour710 | Rat IgG2b, κ | 24DMS1 | 1:500 | eBioscience |
| Anti-human CD44 | BV 570 | Rat IgG2b, κ | IM7 | 1:200 | BioLegend |
| Anti-human CD27 | V450 | Armenian hamster | LG.3A10 | 1:200 | BD |
| | | IgG1, κ | | | Biosciences |
| Anti-mouse CD25 (IL2Ra) | BV 650 | Rat IgG1, λ | PC61 | 1:100 | BioLegend |
| Anti-mouse CD366 | BV 785 | Rat IgG2a, κ | RMT3-23 | 1:200 | BioLegend |
| (Tim-3) | | | | | |
| Anti-mouse IFN-γ | BUV 737 | Rat IgG1, κ | XMG1.2 | 1:100 | BD |
| | | | | | Biosciences |
| Anti-mouse TNFα | BV 711 | Rat IgG1, κ | MP6-XT22 | 1:100 | Biolegend |
| Anti-human Granzyme B | AF700 | Mouse IgG1, κ | GB11 | 1:200 | BD |
| | | | | | Biosciences |
| Anti-human TOX | PE | Human IgG1, κ | REA473 | 1:200 | Miltenyi |
| Anti-human TCF1 | AlexaFlour 647 | Rabbit IgG | C63D9 | 1:200 | Cell Signaling |
| Anti-mouse KI67 | BV480 | Mouse IgG1, κ | B56 | 1:200 | BD |
| | | | | | Biosciences |
| Anti-mouse CD4 | BUV496 | IgG2b, κ | 30-F11 | 1:100 | BD |
| | | | | | Biosciences |

### Results of the Examples

### MDM2-Inhibition Increased Vulnerability of Mouse and Human AML Cells to Allogeneic T-Cell Mediated Cytotoxicity

To test the hypothesis that MDM2-inhibition would synergize with the allogeneic immune response, we treated mice with allo-HCT using bone marrow (BM) alone or in combination with T-cells. In mice bearing myelomonocytic leukemia cells (WEHI-3B), the addition of T-cells to the allogeneic BM graft improved survival (FIG. 1a). Treatment of leukemia bearing mice with MDM2-inhibitor in the absence of donor T-cells improved survival, but did not lead to long-term protection (FIG. 1a). Only when T-cells were combined with MDM2-inhibition were a majority of the mice (>80%) protected long-term (FIG. 1a). A comparable survival pattern was seen in the AML^{MLL-PED/FLT3-ITD} model (FIG. 1b) and in a humanized mouse model using OCI-AML3 cells (FIG. 1c). The T-cell/MDM2-inhibitor combination did not increase acute GVHD severity compared to T-cells/vehicle (FIG. 5a-c).

In vitro cytotoxicity of allogeneic T-cells was higher when OCI-AML3 cells were exposed to MDM2-inhibition (FIG. 1d). Consistently, cleaved caspase-3 was highest when T-cells were combined with MDM2-inhibition (FIG. 1e-f).

To understand the mechanism responsible for the observed in vivo synergism, we exposed OCI-AML3 cells to MDM2-inhibition. Unbiased gene expression analysis revealed upregulation of TRAIL-R1 and TRAIL-R2 by leukemia cells upon MDM2-inhibition (FIG. 1g). Consistently, TRAIL-R1/TRAIL-R2-protein and TRAIL-R1/TRAIL-R2-RNA were increased upon MDM2-inhibition with human OCI-AML3 cells (FIG. 1h-i, FIG. 6a-j), and with mouse WEHI-3B cells with MDM2-inhibition (RG7112, HDM201) (FIG. 7a-h) or MDMX-inhibition (XI-006) in OCI-AML cells (FIG. 8a-c). RG7112 and HDM201 both inhibit p53 degradation by preventing HDM2 binding. We used p53-knockdown OCI-AML3 cells to test whether increased TRAIL-R1/2 expression after MDM2-inhibition was dependent on p53, and found doxorubicin induction of p53 was decreased in p53-knockdown cells (FIG. 9a), while MDM2-inhibition induced p53 in p53-wildtype cells (FIG. 9b). TRAIL-R1/2 expression increased with MDM2-inhibition (RG7112 or HDM201) in cells with intact p53, but not in the p53-knockdown cells (FIG. 1j-k, FIG. 9c-d). Consistently, TRAIL induced less apoptosis in p53^{-/-} AML cells (FIG. 9e). Chromatin immunoprecipitation revealed p53 binding to the TRAIL-R1/2-promoter (FIG. 1l-m).

### Increased TRAIL-R1/2 Expression Upon MDM2-Inhibition Contributes to GVL-Effects

To determine to what extent TRAIL-R1/2 expression in AML cells contributes to enhanced GVL-effects upon MDM2-inhibition, we treated mice with anti-TRAIL-ligand blocking antibody. This reduced the protective effect of the allo-T-cell/MDM2-inhibition (FIG. 2a). Interestingly, the transfer of TRAIL-ligand deficient T-cells (Tnfsf10^{tm1b(KOMP)Wtsi}/MbpMmucs) also reduced the protective effect of MDM2-inhibition (FIG. 2b). Furthermore, in vitro blockade of TRAIL-R1/2 reduced cytotoxicity of allogeneic T-cells towards MDM2-inhibition exposed leukemia cells (FIG. 2c-e). TRAIL-R2 CRISPR-Cas-knockout AML cells (FIG. 10a-c) were less susceptible to the allo-T-cell/MDM2-inhibition effect (FIG. 2f). The therapeutic synergism of TRAIL plus MDM2-inhibition was observed in WT-AML but not TRAIL-R2^{-/-} AML cells (FIG. 2g). T-cells isolated from MDM2-inhibitor treated mice showed higher glycolytic activity measured by an extracellular flux assay (FIG. 2h-i).

Increased glycolytic flux was confirmed by elevated incorporation of U-¹³C-glucose into several glycolysis intermediates (FIG. 2j). In addition, nucleotides and their precursors, in particular of the pyrimidine biosynthesis pathway, were enriched in T-cells isolated from MDM2-inhibitor treated mice (FIG. 11a-c). Increased glycolytic flux and nucleotide biosynthesis are indicative of a stronger T-cell activation, corresponding to higher GVL-activity (6).

### MDM2-Inhibition Promotes Cytotoxicity and Longevity of Donor T Cells

Donor CD8⁺ T-cells displayed higher expression of the anti-tumor cytotoxicity markers perforin and CD107a, and of IFN-γ, TNF, and CD69 in allo-HCT recipients which had received MDM2-inhibitor compared to those receiving vehicle alone, without a total increase in CD8⁺ T-cells (FIG. 3a-h, FIG. 12a, FIG. 13a-b). In naïve mice CD107a, TNF and CD69 increased upon MDM2-inhibition (FIG. 14a-d). Depletion of CD8⁺ T-cells but not NK-cells (FIG. 15a-b) caused loss of the protective MDM2-inhibition effect (FIG. 3i), indicating that the anti-leukemia effect is mediated by CD8⁺ T-cells. To understand whether recall-immunity developed under MDM2-inhibitor-treatment, we isolated donor-type CD8⁺ T-cells from leukemia-bearing mice treated with vehicle or MDM2-inhibitor (FIG. 16a). T-cells derived from MDM2-inhibitor-treated, leukemia-bearing mice caused improved control of leukemia in secondary leukemia-bearing mice (FIG. 3j), indicating an anti-leukemia recall response. Effector T-cells lacking CD27 display a high antigen recall response (12) and we observed a lower frequency of CD8⁺CD27⁺TIM3⁺ donor T-cells in MDM2-inhibitor-treated recipients (FIG. 3k-m, FIG. 17). T-cells in MDM2-inhibitor treated mice exhibited features of longevity (13) including high Bcl-2 and IL-7R (CD127) (FIG. 18a-d).

### MDM2-Inhibition in Primary Human AML Cells Leads to TRAIL-1/2 Expression

To validate our findings from the mouse model in human cells, we studied the effects of MDM2-inhibition on primary human AML cells. MDM2-inhibition increased levels of p53 (FIG. 19a-d), indicating on-target activity. MDM2-inhibition also increased levels of TRAIL-R1 and TRAIL-R2 RNA (FIG. 4a-d) and protein (FIG. 20a-e). The combination of MDM2-inhition and allogeneic T-cells enhanced elimination of the primary human AML cells in immunodeficient mice (FIG. 4e). AML cells exhibited increased TRAIL-R1/2 expression upon MDM2-inhibition (FIG. 21a, FIG. 22a-c). The synergistic effect was dependent on intact p53 because human p53^{-/-} AML cells were resistant to the MDM2-inhibitor/allo-T-cell combination (FIG. 4f, FIG. 23a). The MDM2-inhibitor/allo-T-cell combination caused activation of the TRAIL-R1/2 downstream pathway (caspase-8, caspase-3, PARP) in human AML cells (FIG. 4g).

### Oncogenic Mutations Activating MDM2 Expression Confer Increased Susceptibility to the T-Cell/MDM2-Inhibitor Combination

To identify AML subtypes that may be particularly susceptible to the T-cell/MDM2-inhibitor combination, we studied multiple common oncogenic mutations or gene fusions (FLT3-ITD, KRAS-G12D, cKIT-D816V, JAK2-V617F, FIP1L-PDGFR-α, BCR-ABL and c-myc) for their impact on MDM2. Mice receiving syngeneic BM transduced with the indicated oncogenic vectors developed splenomegaly and BM-infiltration with GFP⁺ transgenic cells (FIG. 24a-c). cKIT-D816V and FIP1L-PDGFR-α induced MDM2 and MDM4 (FIG. 24d-g). Interestingly, the allo-T-cell/MDM2-inhibitor combination after allo-BMT was highly effective in mice carrying FIP1L-PDGFR-α-mutant and cKIT-D816V-mutant AML (FIG. 24h-i).

### MDM2-Inhibition Increases MHC class I/II Expression on AML Cells in a p53-Dependent Manner

Since downregulation of MHC genes and loss of mismatched HLA was shown to cause AML relapse after allo-HCT (2, 4), we tested whether MDM2-inhibition could upregulate MHC molecules on AML cells thereby enhancing their recognition by allogeneic T-cells.

Gene expression analysis revealed upregulation of HLA class I and II upon MDM2-inhibition (FIG. 25a). At the protein level, MDM2-inhibition increased HLA-C and HLA-DR expression on leukemia cells (FIG. 4h-k, FIG. 25b-c). HLA-DR was chosen because HLA-DR-downregulation was shown to be connected to AML-relapse after allo-HCT (2). Consistent with p53-dependent regulation, HLA-C and HLA-DR did not increase with MDM2-inhibition in the p53-knockdown OCI-AML3 cells (FIG. 4l-m). As an approach to increase p53-activity, MDMX-inhibition (XI-006) (14) also increased HLA-C and HLA-DR (FIG. 25d,e). MDM2-inhibition caused increased MHC-II expression on primary human AML cells (FIG. 4n-o) and in AML-cell lines, but not in non-malignant cells (FIG. 26a-l). These findings indicate that targeting MDM2-induced p53-downregulation enhances anti-leukemia immunity post allo-HCT via MHC-II and TRAIL-R1/2 upregulation in mice and humans (FIG. 27).

### Discussion of the Examples

AML relapse is caused by immune escape mechanisms (9). Our recent work has shown that AML cells produce lactic acid as an immune escape mechanism, thereby interfering with T-cell metabolism and effector function (6). A second mechanism leading to relapse is through FLT3-ITD oncogenic signaling blocking IL-15 production, resulting in reduced immunogenicity of AML (5). In this study, we tested a new concept of relapse treatment, combining the alloreactivity of donor T-cells with a pharmacological approach reversing TRAIL-R1/2 and MHC-II downregulation.

We found that MDM2-inhibition induced TRAIL-R1/2 expression in primary human AML cells and AML cell lines. Upon TRAIL ligation, TRAIL death receptors assemble the death-inducing-signaling-complex (DISC) composed of FAS-associated protein with death domain (FADD) and pro-caspase-8/10 at their intracellular death domain (15). TRAIL-R activation was shown to have anti-tumor activity(l6). Furthermore, MDM2-inhibition also increased MHC-II expression in primary human AML cells, which could offer a point for pharmacological intervention to reverse the MHC-II decrease observed in human AML relapse after allo-HCT (2, 3).

Our observation is clinically highly relevant, because leukemia relapse is responsible for 57% of the death of patients undergoing allo-HCT (1, 17). We also delineate the immunological mechanism behind this observation, thereby providing a scientific rationale for using MDM2-inhibition and T-cells to treat AML-relapse, which will lead to a phase-I/II clinical trial.

### REFERENCES

1. D'Souza, A., et al. Current Uses and Outcomes of Hematopoietic Cell Transplantation (HCT): CIBMTR Summary Slides, 2018. CIBMTR Summary Slides Available at: http://www.cibmtr.org(2018).
2. Christopher, M.J., et al. Immune escape of relapsed AML cells after allogeneic transplantation. N Eng J Med 379, 2330-2341 (2018).
3. Toffalori, C., et al. Non-genomic alterations in antigen presentation and T cell costimulation are distinct drivers of leukemia immune escape and relapse after hematopoietic cell transplantation. Nature medicine early online (2019).
4. Vago, L., et al. Loss of mismatched HLA in leukemia after stem-cell transplantation. N Eng J Med 361, 478-488 (2009).
5. Mathew, N. R., et al. Sorafenib promotes graft-versus-leukemia activity in mice and humans through IL-15 production in FLT3-ITD mutant leukemia cells. Nature medicine 24, 282-291 (2018).
6. Uhl, F. M., et al. Metabolic reprogramming of donor T cells enhances graft-versus-leukemia effects in mice and humans. Science translational medicine 12, eabb8969 (2020).
7. Zeiser, R., et al. Mechanisms of immune escape after allogeneic hematopoietic cell transplantation. Blood 133, 1290-1297 (2019).
8. Zeng, D. F., et al. Analysis of drug resistance-associated proteins expressions of patients with the recurrent of acute leukemia via protein microarray technology. Eur Rev Med Pharmacol Sci. 18, 537-543 (2014).
9. Zeiser, R., et al. Biology-Driven Approaches to Prevent and Treat Relapse of Myeloid Neoplasia after Allogeneic Hematopoietic Stem Cell Transplantation. Biol Blood Marrow Transplant. 25, 128-140 (2019).
10. Kojima, K., et al. MDM2 antagonists induce p53-dependent apoptosis in AML: implications for leukemia therapy. Blood 106, 3150-3159 (2008).
11. Vassilev, L. T., et al. In vivo activation of the p53 pathway by small-molecule antagonists of MDM2. Science 303, 844-848 (2004).
12. Schiott, A., Lindstedt, M., Johansson-Lindbom B, Roggen E, Borrebaeck C A. CD27− CD4+ memory T cells define a differentiated memory population at both the functional and transcriptional levels. Immunology 113, 363-370 (2004).
13. van Bockel, D. J., et al. Persistent survival of prevalent clonotypes within an immunodominant HIV gag-specific CD8+ T cell response. J Immunol. 186, 359-371 (2011).
14. Garcia, D., et al. Validation of MdmX as a therapeutic target for reactivating p53 in tumors. Genes Dev. 25, 1746-1757 (2011).
15. Dickens, L. S., et al. The ‘complexities’ of life and death: death receptor signalling platforms. Exp Cell Res. 318, 1269-1277 (2012).
16. Walczak, H., et al. Tumoricidal activity of tumor necrosis factor-related apoptosis-inducing ligand in vivo. Nature medicine 5, 157-163 (1999).
17. Nasilowska-Adamska, B., et al. Mild chronic graft-versus-host disease may alleviate poor prognosis associated with FLT3 internal tandem duplication for adult acute myeloid leukemia following allogeneic stem cell transplantation with myeloablative conditioning in first complete remission: a retrospective study. Eur J Haematol. 96, 236-244 (2016).
19. Wilhelm, K., et al. Graft-versus-host disease enhanced by extracellular adenosine triphosphate activating P2X7R. Nature medicine 12, 1434-1438 (2010).
20. Schwab, L., et al. Neutrophil granulocytes recruited upon translocation of intestinal bacteria enhance GvHD via tissue damage. Nature medicine 20, 648-654 (2014).
21. Zimmerman, E. I., et al. Crenolanib is active against models of drug-resistant FLT3-ITD-positive acute myeloid leukemia. Blood 122, 3607-3615 (2013).
22. Bernot, K. M., et al. Eradicating acute myeloid leukemia in a MII(PTD/wt):Flt3(ITD/wt) murine model: a path to novel therapeutic approaches for human disease. Blood 122, 3778-3783 (2013).
23. Warner, N. L., et al. A transplantable myelomonocytic leukemia in BALB-c mice: cytology, karyotype, and muramidase content. Journal of the National Cancer Institute 43, 963-982 (1969).
24. Bric, A., et al. Functional identification of tumor-suppressor genes through an in vivo RNA interference screen in a mouse lymphoma model. Cancer Cell 16, 324-335 (2009).
25. Brummelman, J., Haftmann, C., Nú\ddot{n}ez, N. G., Alvisi, G., Mazza, E. M. C., Becher, B., Lugli, E. Development, application and computational analysis of high-dimensional fluorescent antibody panels for single-cell flow cytometry. Nat Protoc. 14, 1946-1969 (2019).
26. Hamarsheh, S., et al. Oncogenic KrasG12D causes myeloproliferation via NLRP3 inflammasome activation. Nat Commun. 11, 1659 (2020).
27. Kohler, M., et al. Activation loop phosphorylation regulates B-Raf in vivo and transformation by B-Raf mutants. EMBO J. 35, 143-161 (2016).
28. Kaplan, D.H., et al. Target antigens determine graft-versus-host disease phenotype. J Immunol 173, 5467-5475 (2004).
29. Prestipino, A., Emhardt, A., Aumann, K., O'Sullivan D, Gorantla SP, Duquesne S, Melchinger W, Braun L, Vuckovic S, Boerries M, Busch H, Halbach S, Pennisi S, Poggio T, Apostolova P, Veratti P, Hettich M, Niedermann G, Bartholomä M, Shoumariyeh K, Jutzi J, Wehrle J, Dierks C, Becker H, Schmitt-Graeff A, Follo M, Pfeifer D, Rohr J, Fuchs S, Ehl S, Hartl F A, Minguet S, Miething C, Heidel F, Kroger N, Triviai I, Brummer T, Finke J, Illert AL, Ruggiero E, Bonini C, Duyster J, Pahl H L, Lane S W, Hill G R, Blazar B R, Bubnoff N, Pearce E L, Zeiser R. Oncogenic JAK2V617F causes PD-L1 expression mediating immune-escape in myeloproliferative neoplasms. Sci Transl Med. 10, eaam7729 (2018).
30. Pang, Z., Chong, J., Zhou, G., Morais D., Chang, L., Barrette, M., Gauthier, C., Jacques, P E., Li, S., and Xia, J. MetaboAnalyst 5.0: narrowing the gap between raw spectra and functional insights. Nucleic Acids Res. doi: 10.1093/nadg/kab382(2021).
31. Antoniewicz, M. R., Kelleher, J. K., & Stephanopoulos, G. Accurate assessment of amino acid mass isotopomer distributions for metabolic flux analysis. Analytical Chemistry 79, 7554-7559 (2007).
32. Buescher, J. M., Antoniewicz, M. R., Boros, L. G., Burgess, S. C., Brunengraber, H., Clish, C. B., et al. A roadmap for interpreting 13C metabolite labeling patterns from cells. Current Opinion in Biotechnology 34, 189-201 (2015).

1. Mouse double minute 2 (MDM2) inhibitor for use in the treatment and/or prevention of a hematologic neoplasm relapse after hematopoietic cell transplantation (HCT) in a patient.

2. The MDM2 inhibitor for use according to claim 1, wherein the hematologic neoplasm is selected from the group comprising leukaemia, lymphomas and myelodysplastic syndromes.

3. The MDM2 inhibitor for use according to any one of the preceding claims, wherein the hematologic neoplasm is a leukaemia, preferably acute myeloid leukaemia (AML).

4. The MDM2 inhibitor for use according to any one of the preceding claims, wherein the HCT is an allogeneic HCT.

5. The MDM2 inhibitor for use according to any one of the preceding claims, wherein the HCT comprises T cells.

6. The MDM2 inhibitor for use according to any one of the preceding claims, wherein the inhibitor is administered to a patient after HCT and before occurrence of a relapse.

7. The MDM2 inhibitor for use according to any one of claims 1-5, wherein the inhibitor is administered to a leukaemia patient after occurrence of a relapse after HCT.

8. The MDM2 inhibitor for use according to any one of the preceding claims, wherein the inhibitor is selected from the group comprising RG7112 (R05045337), idasanutlin (RG7388), AMG-232 (KRT-232), APG-115, BI-907828, CGM097, siremadlin (HDM-201), and milademetan (DS-3032b), and pharmaceutically acceptable salts thereof.

9. The MDM2 inhibitor for use according to claim 8, wherein the inhibitor is siremadlin (HDM-201), or a pharmaceutically acceptable salt thereof.

10. The MDM2 inhibitor for use according to any one of the preceding claims, wherein administration of the MDM2 inhibitor leads to upregulation of one or more of TNF-related apoptosis-inducing ligand receptor 1(TRAIL-R1), TRAIL-R2, human leukocyte antigen (HLA) class I molecules and HLA class II molecules.

11. The MDM2 inhibitor for use according to any one of the preceding claims, wherein the treatment further comprises administration of an allogeneic T cell transplantation, either together with the HCT and/or after HCT.

12. The MDM2 inhibitor for use according to claim 11, wherein the allogenic T cell transplantation is a donor lymphocyte infusion that comprises lymphocytes, but does not comprise hematopoietic stem cells.

13. The MDM2 inhibitor according to claim 11 or claim 12 wherein the donor of the allogenic T cell transplantation was also the donor of the HCT.

14. The MDM2 inhibitor according to any one of claims 11 to 13, wherein the MDM2 inhibitor is administered after the HCT, and before and/or the same day as, and/or after administration of the allogenic T cell transplantation.

15. The MDM2 inhibitor for use according to any one of the preceding claims, wherein administration of the MDM2 inhibitor increases cytotoxicity of CD8+ allo-T cells towards cancer cells, wherein preferably cytotoxicity of CD8+ allo-T cells is at least partially dependent on interaction of TRAIL-R of the cancer cells and TRAIL-ligand (TRAIL-L) of the CD8+ allo-T cells.

16. The MDM2 inhibitor for use according to any one of the preceding claims, wherein administration of the MDM2 inhibitor increases a graft-versus-leukaemia or a graft-versus-lymphoma reaction, preferably wherein the graft-versus-leukaemia reaction or the graft-versus-lymphoma reaction is mediated by CD8+ allo-T cells.

17. The MDM2 inhibitor for use according to any one of the preceding claims, wherein administration of the MDM2 inhibitor increases expression of one or more of perforin, CD107a, IFN-γ, TNF and CD69 by CD8+ allo-T cells.

18. The MDM2 inhibitor for use according to any of the preceding claims wherein the treatment further comprises administration of an exportin-1 (XPO-1) inhibitor.

19. An XPO-1 inhibitor for use in the treatment and/or prevention of a hematologic neoplasm in a patient wherein the treatment further comprises administration of a haematopoietic cell transplant and an MDM2 inhibitor.


SEQUENCE LISTING (16 sequences)

SEQ ID NO: 1 - DNA, 22 bp, Artificial Sequence
  Primer forward hTrailR1
gtgtgggttacaccaatgcttc

SEQ ID NO: 2 - DNA, 22 bp, Artificial Sequence
  Primer reverse hTrailR1
cctggtttgcactgacatgctg

SEQ ID NO: 3 - DNA, 21 bp, Artificial Sequence
  Primer forward hTrailR2
acagttgcagccgtagtcttg

SEQ ID NO: 4 - DNA, 20 bp, Artificial Sequence
  Primer reverse hTrailR2
ccaggtcgttgtgagcttct

SEQ ID NO: 5 - DNA, 21 bp, Artificial Sequence
  Primer forward CDKN1A
gtggctctgattggctttctg

SEQ ID NO: 6 - DNA, 20 bp, Artificial Sequence
  Primer reverse CDKN1A
ctgaaaacaggcagcccaag

SEQ ID NO: 7 - DNA, 20 bp, Artificial Sequence
  Primer forward TNFRSF10A
ttcgcattcggagttcaggg

SEQ ID NO: 8 - DNA, 20 bp, Artificial Sequence
  Primer reverse TNFRSF10A
aagtggcaaaacgactccga

SEQ ID NO: 9 - DNA, 18 bp, Artificial Sequence
  Primer forward TNFRSF10B
acgactggtgcgtcttgc

SEQ ID NO: 10 - DNA, 21 bp, Artificial Sequence
  Primer reverse TNFRSF10B
aagacccttgtgctcgttgtc

SEQ ID NO: 11 - DNA, 22 bp, Artificial Sequence
  Primer forward GAPDH
gtctcctctgacttcaacagcg

SEQ ID NO: 12 - DNA, 22 bp, Artificial Sequence
  Primer reverse GAPDH
accaccctgttgctgtagccaa

SEQ ID NO: 13 - RNA, 29 nt, Artificial Sequence
  TRAIL-R1 targeted shRNA
ttcgtctctgagcagcaaatggaaagcca

SEQ ID NO: 14 - RNA, 29 nt, Artificial Sequence
  TRAIL-R2 targeted shRNA
agagacttgccaagcagaagattgaggac

SEQ ID NO: 15 - RNA, 29 nt, Artificial Sequence
  non-silencing shRNA control
gcactaccagagctaactcagatagtact

SEQ ID NO: 16 - RNA, 20 nt, Artificial Sequence
  Trail-R2 gRNA design
cgcggcgacaacgagcacaa